SEQUENCE LISTING (45 sequences)

SEQ ID NO: 1 - DNA, 7038 bp, Homo sapiens
  source 1..7038: mol_type = genomic DNA
gctcctttctctctcacgataaactcaatggggttgtctatttttaacctcctgttctttccttttggcagagtaatcagtgtgagctgagctctttccctgaatgaatccaagtaaccctggccttctgaaatgaccagcagacacacaactgtccagaggctgcccaaagtataaactctggttctaaagtaccatgtccagccaaggaagtcctagtgttgcgctttctacgacgacagtcagttcagttgctgtgcaggctggggactccaaaatcgttatagctgtcataaagtgtggcaaatgggtacaacttcaactggctgaatcacagcccaatcttctagaaattggcagcagtcaagatgaaaccaaaaaacttcttcatgatcatgaacttcttttggccaagctcaaggctttggaagatcgggtatgggaactcttgcaggaagcagacaagacagctgaagagaacaaggatcagagtcaggtctatgatgccatggccgagactctgggtgaagcatgggcagctctggtgtccatgcttgaaagaagaacagagctccttaggttgacttctgaattttttgaaaatgccttagagtttgctattaaaatagaccaagctgaagatttcctccagaatactcatgagtttgagagtgctgagtccttaaaatcacttcttcagcttcatgaacatcatactaaagaactcttggaacggtctttagcccttttaaacaaaagtcaacaactcactgacttcatagaaaaattcaagtgtgaaggacctaatgtgaatcctgagttgactcagggagctcatagcagctgtctgaaggttgaccgccttcttgaacttctacaagacaggagaagacaactagacaagtacttgaagcaacagtggcaagaattgagtcaagttctgcagatatgtcagtgggaccaacaagaaaaccaggttacttgttggtttcagaaaactataagaaatttacaggaacaaagtctaggttcatcactttcagacaatgaggatcgaattcataagcaagaggaactgataataaaagcaaaggaatggaattctgctgttgagaagctgaagagtgaggcactgagaattctgctgtcaaaggactacgtggagaaagaacacctccagctctctcaccagaaactcagtcagcttcaagaagaatttggtcaactcatggtggaaaggaatacctggttaaagaaggcgaatgaattttttaacagtgctaacaaggcatttgatgtacttggaagagttgaagcttaccttaagctccttaaatcagagggtttaagtctggctgttttggcagtgaggcatgaggaattacacagaaaaattaaagactgcacaactgatgctttgcaaaagggacaaaccttaatcagccaagtagactcctgcagctctcaggtgtccggcatccatgagatgatggggtgcattaagagacgagtggatcatctgaccgaacagtgttcagcgcacaaggaatatgctcttaagaaacaacaactaacagcctcagtggagggttacctacggaaggtggaaatgtcaattcagaaaatcagtccagtactttctaatgcaatggatgttggttctacccgttctgaatcagagaagattttgaataaatatctggaactagatatccaagctaaggagacatcacatgaattagaagcagctgcaaaaaccatgatggagaaaaatgaatttgtatctgatgaaatggtatcactttcctctaaagctagatggctagcagaagaattaaacctatttggccaaagcattgactatagatcgcaagtcctgcaaacttacgtggcatttctgaagtcatcagaggaggtagagatgcagtttcagagcttaaaagaattttatgaaaccgaaatccctcagaaggagcaggatgatgctaaagccaagcattgttctgactcggctgagaagcagtggcagctatttttaaagaagagttttataacacaagatctagggcttgagttccttaatttaataaatatggtaagaatggcattaaatggatcacaccttctgcagggatactcatcaaagtcaaaataataatttcaaaggttcagctgtctattaatcagcagttctctcctagggagttctctttagcttcattcaacctagaccagtcctgaagcccaacatttaaatgcttgcttggcctagaactttggttagtttataacaggaaggctgttttaactattaagaagaaagtgactgtaatccaaatgcaacattgttattattaaatagagtcctcagaagtccacccaaaagttctgtgttattttgaaagcatgcaacaaggtcctttcaaaatttctaggggctaaagacaacttctaagatgaatcttcttttgaagagtttcttaccatagcagatctctttgtagtgaagatccattgcatttagaaaaataccctaacctttaactgtcttgaattatatctgtaaaatattttagaatccttatatccacccataacccatataaaattccctgtaggatacttaaaagatagtccacttctctttcaatacctcaagtattttcctaatgaaactgctcatttgattgttggagaatgccagctgctagaaaccttctttttttaatgttaaatatatctgtttccgtatctactcctaatcatctgtagctacacagggacaaaaagtattcatatttaatttctcttacacatgaaagtccttccaacattaaaaacagttgctatgtctgagttttttctgcagcattaaaaatcctttccatcatttatgaaggagctttatttttctttctatgctgatccccccttctcaggattttctatggtttgtcaagatcattcattaaatgtaaccaaaaagtgaactacaggtgcaacacagagtacagtaaactggacattattattcactaatgagacctacaattatgctaacatttttagcagtagtttcactttgctggtacatattaagcttgtgatagaacaaaactctcataatatttttattgcagcttctgtcaaaccatgtctctcccatattatacctgtgtcagccatttaccttttttatacaaatgtgggcctttatattttttcttatttaattttattttgttagtcttcacacatgatcccatcttgctaaactaatttgaacctacctagtatattagccatctgtcttgggtttaattctgtagatatgacaagtaagctttctatggcagcaattagttcttaaatataaatgcctaaagggcagattggcattccagttcatgaagcagtctattgattaaaccagtttttaaaataatcatgaatgctcccaatagtacttcattcagccgctacttcataatctttgtattaagtctataatgggagactttctcaaataaactgctcaaaagaaggtttacctacctagtatcttatcaatgacatagacaatttgacataaatcaggttggctcctagcgatcaaggcattctttccaaaatgctcacaaagtgcttgataagtaatgaacttattgcactgattttcagtgtgaagcttaccaatctgtaaattgtgtagtccatcctttcctccttttgaaaatcagggcattttctgttctctaattttatggctcttctagtttctgtagattttcaaagataactgacagctgcaattttctctgtacctgggatgtgattaatctcagtctggagcctgaattcatttggaagagttaggttctaactaccctcatctacttggagctgcaattttctctttatcttgtttcaacttttgttgtttgaagaccattcttgataaacctacttggaaataaaatatgcattagctgattctcatttattcaacatgattcttaagcactaaagaaaacttgctaagcaatttgcccatgttcctatgtgatctaggtgctttactgcaaaaaaaaagaaaagttatttttcgtttagaacatttagaacatgtattatctatctatctatctctgtctctctctcacacttctacgtggttcaatcagtcttctcccatctgttgatgatgtatgtttctgatttacctaagggaattcaaatactttcaaactgcgtaattttgtctacctttttattactaacttcatttcagaaattttagttctggaatctacagtcggtacttttatttattaaattactgatgaaattctgtttcttgtctgctcttttcccatctttccaatactttcttgaactatattaaccctaggcacttaaagtgttcatcagctagctacaacatttgtacctggagctctttctttctttctttcctttcttttcttttttttttagatggagtctcgctctgtcacccagactggagtgcagtggtgcgatctcagctccctttaacctccagctcctgggttcaagcaattctcatgcctcagtcccccgattagctggaattacaggtgcctgccaccatgcccggctaatacttgtatttttagtagagacgaggtttcaccatgttgaccaggctggtctcaaactcctgacttcaagtgatctgcccacctctgcctcccaaagtgctgggattacaggcatgaaccacagcgcccggccaatctggagctgtttctgttgtctaatttttctcttggtttccagtcctgtagtcctgtttcttggcagaactagcgaatatttatcaaatgccagacattatgtataaaaaattggaggatcagcagatggtgttttcttcttctagaaagggtcacccttctttctccaggcaggcattggtgggcaatcactcaagccaatcagaagctggcagagttgtgtttgcagtgtagtcctgtcccctcaggctcccagagttttgtattgagagccaagtgctttctctgacgtctctgcccctggcaggtcacaaactccaatctttgtttgggtagtctgctgaaaaactccactctgcttttcagaggccttttgcttaggttttttaatccactgcccgatatacccctggtattttgtcaacgtctttagaagatcattgtgcttgaagccctccaagtgtctgccaaaatctctggtgttttctcggctccagggaagtctcgattatcagcttcctgtctgcacagaattgggaaatccccctgggatgataggtgagctcacccctgtcaggttcttccctctctggagtcagcctctctagttccggttgtccgaccagttctctgcttccttcaaatggattccttctgcattttacccagcttttctaggtattctaagcaaaagcactgctctgccactggctaccccattctactcagaagtcctttgttacttatttagaatgtttaagattgtacagtcagatttttgtttttaaagatctgcaacaataacttattccaaattatattttgtatctatatctaaataaacttaggttatcataatattcaatgcccacttagatctcccagaaatagcatatattttactactgtgctttggtgttcttttatcttgatgtcataaacacggtcactctaccctaaggtctttaccactatcatatgataaataagttcctccttattgataaaattaaagcccacgtactaaggaaatttattggttagcaacatttggagagatgaaattgttggtaaagtaagtaaaacgattttttttattcttatatcttttggaccactaggcttttaaacaaacattattgttgaatcatggtgactagtttgcttgctaagaaggcagaggccagctggtatattcagttgagtgttctgttctttattgggttgtcgtggctcatattttcaagccagaaatagaaaaaatttcagttcatctaaaaatcattaagatcagagaaactgattcaggcccaaattatggcagacatcttgatgaaagtgctaatttattaatttataatggccaataccaagaatgctatctgggatacagccttcctattatcaaggtatctgaaaagcaagacatcagaaaaccattcatggtgcatgaatataaacgaaaaatgaatgctttagtggcctgtttagactggattatgacaaacattttccatgtcctacacagaagtaaaatgagtactgtgtttactaattagacctcaggcaaattattcaatcttcttgagtggccaaaatattcgtctgccaaatacatgatcacttttcacagatcctactagctagacatctacaaatcctactttagctctcctttctctagacctcactcagaccaccatatccttctgattaaagttgtcattggcatgctgcagtttcaaaatttatattaaattatataatacattacattctggttaatcatttctttaaaatatgtgaaagataattttatttatgataaaagtataatttggggtgaattctgctgtagttgtctaatatctttcataatttattatgtatcattattaataatgagggaatataacgttttattactagattcttaagttcatgggggaaatctcatttaggacctacagatatatatttttttggagttgccactttataatgtgtaaaataacataaaaatataagtgtgcaacatcaatatcatttaaaataattttaaaataaaatggcctatctttaaaatatcctgttaagtatttatgatactgtcaattgctgtcattgagatgatagaaatttgaagatttattgagaaagcctggctattaaagtaaaaaacataagaattgtttcgccaaatgaatgttcctgtgcattttttaaatgtttctacagaaataaagcacgcatagtctta

SEQ ID NO: 2 - DNA, 6329 bp, Mus musculus
  source 1..6329: mol_type = genomic DNA
tgttctttccttttggccgggcaatcagtgtgaacagaggcgcgttccctgaaagagtcccggtagcctcgtggaagccttctgacatgaccagcagacagcatcactaccaaggctgctcggggtttaagccctgcttctaaagtgccatgtcctgcaaagaaagtccccatgtgggtgcttccaccaccactgtcagctctgtagccgtgcacgctggggactccaagatcgtgatcgccgttgtcaagtgtggcaaatgggtacgactccaactggctgaatcgcagcccaatctcctcgaaattgggagcagtcaagatgaaaccaaaaaactccttcacgatcatgagctgcttctggccaagcttaaggccttggaagatcgtgtgtgggaactcttacgggaagcagaccggacagctgaagcaaacaaggctcaaagtcaggtgtatgacgccatggcccagacgctgggtgaggcatgggcaaccctggtctccatgcttgaaagaagaagggagctccttgggctgacgtccgagttctttcaaagtgctttggagtttgctataaaaatagaccaagctgaagcttttctgcagaatcctcatgagtttgagagcactgaagccttacagtcacttcttctgcttcatgaccgacatgccaaagaactcttagaaagatccctagaccttttaaacaaaagtcaacaactgactgacttcatagaaaaattcaagtgtgaaggatctactatgaattctgagctgatccagggagctcagagcagctgtctgaagatcgacagcctccttgaacttctgcaagacaggagaaggcagctcgacaagtacttgcagcaacagcggcaggaattgtctcaggttctgcagttgtgtctgtgggaccaacaagaaaaccaggtttcttcttggtttcagaaagcaataagagatctgcaggaacagagtctgggcgcatcactttcagacaacagagagctaatctgcaagcacgaggacctgatagtcaaagcaaaggaatgggattcggccgtagagaagctgaaaagccaggctttggggattctattgtccaaggacttggcaggaaaggaacatctacagctctctaaccagaaactgaatcggcttcaagaagaatttggaagactcatggtggaaaggaaagcctggttatcgatggcaaatgacttttttaccagtgctaacaagtcatttgatgtacttgggaaagttgaagcttaccttaagctccttaaatcagagggtttaagcctgcctgtcttggcagcgaagcatgaggaattacacagagaaattaaagactctacagccactgctctgcaaaagggacgaaccttaatcagccaagtagactcctgccgctctcgggtgacaggaatccacgagatgatgggatacattcagaaccgagtggattgtctcactgaacagtgtacagcgcatgaggaatttgcccgcaagagacagcaattagcaacatcagtggacgactacctaaggaaggtggaaatgtcaattcaggaaatcaggcccattcttgctaccacattggatgttgcttccagcccttctgaatcagagaagattttaaataaatatctggaattggatatccaagtcaaggagacagcacatgcattagaagcagctgcaaaaatcatgacagagaaaaatgaacttgaactcaatgaagtggcattgcttcctcttaaagtgaaatggctagaggaagaattaagcacgcttggtcgaagcatcagctgcaggtcacggatcctgcagacgtacgtggcatttcgaaagtcatccgaggaggcagaggagcagcttcagagcctaaaggaattttatctgactgaaattccttggaaagatgaagacgatgctgtggtcaagtgccagtctaactccgctgagaggaaatggcagctgtttttaaagaagagttttttgacccaggacttgagcctcgagtttctgaacttaataaacatggcaaaagagaatgagatattaaatgttaaaaatgaaatgcacattatggagaacattatggaaaagcaaacaaatggaagggaagagctcagccatctccgagtggcgtggtacctcaaagctatcgaaggcaagcctgcgagagagcagtgggaaatgttcaaagagaaacttacaaagactactcacagtgtaaaacttctccacgaagttctcatgcctgtctctgcgcttgaccttggggggagcctccagagcacgtcagatctgcggaggaggtggatcgcaatgaagcctcagctccagcaactgcatgaggatgttcagcaaatcacgaaagagtgggaagtgttaagcagtcagggagctcctctgaaggagaaggctgaacagctgaaggaccttgtccatctccaccggaggcagcgagagcgaatccaggaatatgaggaaatcctatacaagacagtccagttccaccaggtcaaggaagagctggtccatctcatcaaaccaagagaactggaacttctagcacagcccatggaactggcaagttctgaggaggtccagatgcaactgggccgttcgcaagaaaggcgggcacatgtagaccatctccatcagctggccctgaccttaggagtggacatcatttcatccgtgcaacaacctaattgctctaatatttctgcaaagaacctgcagcagcagctggaggcccttgaactggagagcaggagttggagtgcccaagccaaagaacatgagcgcgtcctgtcctgcagcttggagtactgcactgcacgtgatgagatcagtgagctcaaagagtcattcaaagacataaaaaagaaattcaacaatctgaagtttaattactccaagaaaaatgagaaatctcgaaatttaaagactcttcagtatcaaatccaacaagtggatacatatgctgaaaaaattcaggctctaaggaagaaaatggaaaaagttaataataagacatctgattcattcttaagttatccaagtaataaagctaacatgctttcggaagccatggaggatttgaaaaagaacgtggatgactttgacaaagttgtgacagattacaaaatgaatttggacctgactgagcatcttcaggaagtaatagaagagtgtaacttttggtatgaagatgcaagtgccaccgttgtgagagttgggaagtattccatggagtgccaaacaagggaagctgtggacatcctccacaggcagttcaataagtttattactccctcggtgccccagcaagaagaaaggattcaggaggtcattgaccttgctcagcgcttatatggtttggaagaggggcagaaatatgcagagaagattgtcacaagacacaaggagattcttgaatctattactgaattatgtgggtctctcgtggaacttaaagaaaaactgatgcagggagaggttccaaagatgaattcagatttggaagatttccatgacaattgcattgatctgctaaaggggccagggaaggatgaccagaaaacattcagtgaagaaaggaatgaggggcaggtgcagggagcagatgtattggccgtcaatggaacaagagaagatgggcttcccatggacttgaggcggacctcctctgacaaagaggacagcgcccagggtctgatccttccagaagacacactatctggagaagaatctgagtgtatctcatccgatgacatctctttgcctccactgccagtgagccccgagtcccctctggcaccatcagacatggaggtagaagaacttaccagctcctctgccctcgccctgcacatcagcggctacaggatgcatccagggacaggtggtctaggaaaggcccaggagtctgctcttccctcacccattgcctttgcggatgggggccatagtaagaaagacacatttacaagtcattttgagaggccctatccacagttgaaagctgaatcctcattagcctccagaggatcagcagagatgagtaccaagttgcacatcaatgtgaaatgtccagcgagcatgccccatgaagtgcacgacaaagctttacagcaatgctcccaggctcgggagagcacactagaaatgcaggaaaaagtgcatgctgattctaacgtcactaaaacccaagacaggctgcatgctgcccttgatgtgtcccctggcctcggctctcaaccagacaccagcgagagccatcagaggcgagtgggtccccaaggaaacaagaaaaactcatccgcagaaaacagcgtggtcagcctagctggccaggcacctcatttctccaggctgctgtccaatgtaactgtcatggaaggttccccagtgactttggaagtcgaagtaacaggattcccagagcctacactgacatggttcaagaagggccagaaactgtgtgcagatggacacttacaggtcttgcacaaggacacaaagcattcggtgttcattccgaaggtatgtgaggcagatgctggcctctatgtggctcaagcccaaaactctagtggcaccctgtcttccaaagccatcctccatgtgacaggtaaccatgggccaccaattacaagactaaactggattatgctgtgtatcatctatgtcagtgtatcagtaatatactggctacttacacgatagtgacatgttgccatcaatggacattgttatgagcctaaaagacaatatgattgttgattttctcatacatcacacaaagcactcccccaacatacacacaccaatttatctccactttgactaatcccactgctaggtgaaatagttgtattttctctacgatttaacatacttgggaaacacgggaaattatgtacagcgggtaatgatacatttcatactctaggaagagaaagattctgtctaataagctgatctcttgtttaaaggcatatcaatgcgcacatagttcacaaggatcatagggttttgtcctaattttcaaatccaaatgtttttcatttctccttcactgtgctcataactatctctttgtagggcattgcatcaaactgaggttgtgattctcaatgttgtattagcttccattaactgtgcttttcacattttgatcttgtttaccaaacaaacttgacctatgctgtctcaacctactctacattcctacgtcggaggatgtcactatcaataaccattcctcatgatggtctcttgggcttgatgttggagaacagacgtgccatcagcttcaaaatggagtcaacctttctattagctctgaatgtcatatgtcaaaatgacatacaggctgcaggaggggaccagttttccccctgtccttccactgctgtgcattgaccatctgtgcataactataggcaggtcctactgcatctgctgctgctgctgttgctgctgaacactgtgtagccctttacaaggcatctcagtgccccccctttcccatctggaagaggggctcatctacctcttgacaactgagtttttattttatgtcctgtggtgatctggccaatggaaaaatactcagagaagactcattacaatgcttggtgtgatttgccatcatgctactcattacccattaattccctttcagattctgatcttttagaatattaaatgaaaagagtgctgttaatatgtaatttctaactactcccttgggggagggtaagactgatgttctatgaattagagtttaaattcctttctaacttcttacaactacatccttttcaaaggctctggacggtaacctctggaggatatagttagcgtgttttagagggagtctgggtaagatagaagaagacaattctagctttcttttgtgcaattatctctgtaatattgaagaatgttgtatggcttcctaatacgaattattaaaaaaaataagagaggtgtttgttttcatattttaaactttgtcatgctagggtaaaatcaagttgaaagaataatttattacagatttttgatgaagggaagaaattaattatgttgctgaaagactgtctacaaataatcaaaatatttaaatagaatatatttgatgtgatattaaaaagctactgaatgttgtggaagataagaaatgtctagtctagtaaaaaattggcctattttttcttgtcctgtgtatacaatttaattgaaaaattgtaactgttttctttcaagttttgacagaaagctattaaaatcttatatatacac

SEQ ID NO: 3 - protein, 652 aa, Homo sapiens
MSSQGSPSVALSTTTVSSVAVQAGDSKIVIAVIKCGKWVQLQLAESQPNLLEIGSSQDETKKLLHDHELLLAKLKALEDRVWELLQEADKTAEENKDQSQVYDAMAETLGEAWAALVSMLERRTELLRLTSEFFENALEFAIKIDQAEDFLQNTHEFESAESLKSLLQLHEHHTKELLERSLALLNKSQQLTDFIEKFKCEGPNVNPELTQGAHSSCLKVDRLLELLQDRRRQLDKYLKQQWQELSQVLQICQWDQQENQVTCWFQKTIRNLQEQSLGSSLSDNEDRIHKQEELIIKAKEWNSAVEKLKSEALRILLSKDYVEKEHLQLSHQKLSQLQEEFGQLMVERNTWLKKANEFFNSANKAFDVLGRVEAYLKLLKSEGLSLAVLAVRHEELHRKIKDCTTDALQKGQTLISQVDSCSSQVSGIHEMMGCIKRRVDHLTEQCSAHKEYALKKQQLTASVEGYLRKVEMSIQKISPVLSNAMDVGSTRSESEKILNKYLELDIQAKETSHELEAAAKTMMEKNEFVSDEMVSLSSKARWLAEELNLFGQSIDYRSQVLQTYVAFLKSSEEVEMQFQSLKEFYETEIPQKEQDDAKAKHCSDSAEKQWQLFLKKSFITQDLGLEFLNLINMVRMALNGSHLLQGYSSKSK

SEQ ID NO: 4 - protein, 1531 aa, Mus musculus
MSCKESPHVGASTTTVSSVAVHAGDSKIVIAVVKCGKWVRLQLAESQPNLLEIGSSQDETKKLLHDHELLLAKLKALEDRVWELLREADRTAEANKAQSQVYDAMAQTLGEAWATLVSMLERRRELLGLTSEFFQSALEFAIKIDQAEAFLQNPHEFESTEALQSLLLLHDRHAKELLERSLDLLNKSQQLTDFIEKFKCEGSTMNSELIQGAQSSCLKIDSLLELLQDRRRQLDKYLQQQRQELSQVLQLCLWDQQENQVSSWFQKAIRDLQEQSLGASLSDNRELICKHEDLIVKAKEWDSAVEKLKSQALGILLSKDLAGKEHLQLSNQKLNRLQEEFGRLMVERKAWLSMANDFFTSANKSFDVLGKVEAYLKLLKSEGLSLPVLAAKHEELHREIKDSTATALQKGRTLISQVDSCRSRVTGIHEMMGYIQNRVDCLTEQCTAHEEFARKRQQLATSVDDYLRKVEMSIQEIRPILATTLDVASSPSESEKILNKYLELDIQVKETAHALEAAAKIMTEKNELELNEVALLPLKVKWLEEELSTLGRSISCRSRILQTYVAFRKSSEEAEEQLQSLKEFYLTEIPWKDEDDAVVKCQSNSAERKWQLFLKKSFLTQDLSLEFLNLINMAKENEILNVKNEMHIMENIMEKQTNGREELSHLRVAWYLKAIEGKPAREQWEMFKEKLTKTTHSVKLLHEVLMPVSALDLGGSLQSTSDLRRRWIAMKPQLQQLHEDVQQITKEWEVLSSQGAPLKEKAEQLKDLVHLHRRQRERIQEYEEILYKTVQFHQVKEELVHLIKPRELELLAQPMELASSEEVQMQLGRSQERRAHVDHLHQLALTLGVDIISSVQQPNCSNISAKNLQQQLEALELESRSWSAQAKEHERVLSCSLEYCTARDEISELKESFKDIKKKFNNLKFNYSKKNEKSRNLKTLQYQIQQVDTYAEKIQALRKKMEKVNNKTSDSFLSYPSNKANMLSEAMEDLKKNVDDFDKVVTDYKMNLDLTEHLQEVIEECNFWYEDASATVVRVGKYSMECQTREAVDILHRQFNKFITPSVPQQEERIQEVIDLAQRLYGLEEGQKYAEKIVTRHKEILESITELCGSLVELKEKLMQGEVPKMNSDLEDFHDNCIDLLKGPGKDDQKTFSEERNEGQVQGADVLAVNGTREDGLPMDLRRTSSDKEDSAQGLILPEDTLSGEESECISSDDISLPPLPVSPESPLAPSDMEVEELTSSSALALHISGYRMHPGTGGLGKAQESALPSPIAFADGGHSKKDTFTSHFERPYPQLKAESSLASRGSAEMSTKLHINVKCPASMPHEVHDKALQQCSQARESTLEMQEKVHADSNVTKTQDRLHAALDVSPGLGSQPDTSESHQRRVGPQGNKKNSSAENSVVSLAGQAPHFSRLLSNVTVMEGSPVTLEVEVTGFPEPTLTWFKKGQKLCADGHLQVLHKDTKHSVFIPKVCEADAGLYVAQAQNSSGTLSSKAILHVTGNHGPPITRLNWIMLCIIYVSVSVIYWLLTR

SEQ ID NO: 5 - DNA, 3251 bp, Homo sapiens
  source 1..3251: mol_type = genomic DNA
agaagcggccgcaggggtctgggcagggctgggcagtgctgccggagcaaaagcggtagcgggagcccggccggagctgggtctggagacgccgtggcagcctgaacggagtgtgcgacggattgggaggtttgtctacagattttgagcgttcgaagttgacccctgactaagtatactttgctgctccctcagcctttgaaaaaatgtctgtcacatatgatgattccgttggagtagaagtgtccagcgacagcttctgggaggtcgggaactacaagcggactgtgaagcggatcgacgatggccaccgcctgtgcagcgacctcatgaactgcctgcatgagcgggcgcgcatcgagaaggcgtatgcgcagcagctcactgagtgggcccggcgctggaggcagctcgtggagaaagggccccagtacgggaccgtggagaaggcctggatggccttcatgtccgaggcagagagggtgagcgagctgcacctcgaggtgaaggcctcactgatgaacgatgacttcgagaagatcaagaactggcagaaggaagcctttcacaagcagatgatgggcggcttcaaggagaccaaggaagctgaggacggctttcggaaggcacagaagccctgggccaagaagctgaaagaggtagaagcagcaaagaaagcccaccatgcagcgtgcaaagaggagaagctggctatctcacgagaagccaacagcaaggcagacccatccctcaaccctgaacagctcaagaaattgcaagacaaaatagaaaagtgcaagcaagatgttcttaagaccaaagagaagtatgagaagtccctgaaggaactcgaccagggcacaccccagtacatggagaacatggagcaggtgtttgagcagtgccagcagttcgaggagaaacgccttcgcttcttccgggaggttctgctggaggttcagaagcacctagacctgtccaatgtggctggctacaaagccatttaccatgacctggagcagagcatcagagcagctgatgcagtggaggacctgaggtggttccgagccaatcacgggccgggcatggccatgaactggccgcagtttgaggagtggtccgcagacctgaatcgaaccctcagccggagagagaagaagaaggccactgacggcgtcaccctgacgggcatcaaccagacaggcgaccagtctctgccgagtaagcccagcagcacccttaatgtcccgagcaaccccgcccagtctgcgcagtcacagtccagctacaaccccttcgaggatgaggacgacacgggcagcaccgtcagtgagaaggacgacactaaggccaaaaatgtgagcagctacgagaagacccagagctatcccaccgactggtcagacgatgagtctaacaaccccttctcctccacggatgccaatggggactcgaatccattcgacgacgacgccacctcggggacggaagtgcgagtccgggccctgtatgactatgaggggcaggagcatgatgagctgagcttcaaggctggggatgagctgaccaagatggaggacgaggatgagcagggctggtgcaagggacgcttggacaacgggcaagttggcctatacccggcaaattatgtggaggcgatccagtgatgagtcggggacaggccagcggggggacggaggcggcgggcccaggagcctcagccagccacgtgggcatccactccttttcctgcaagagatgatggttccattgctcttggcttcatggtgttcctggaaggcagatgagctggtcatttcgcctgggactcggcacctttccgagtgcagctggagggatctgagcgcaggaagacgcagaacaacagaaatagccgcccctccccgcccactgtgcctgttggcctatcatagatctctatgttcttgactttgtctctcctttccgagtcaatggtgggttacactgatcttgttccactgattactctctctgacgagtccatcacctgcaacttaaatgaacaagcttacatcccattttgagtgaagattttgaggtttttaatttaaaggctgtgtacagttatacttttttatacacctgttcatttctacttaaattatggcacagattgatgcgcaccagtcttgaggaaacgatctccctattcccttaccctgttactcagccacgccgtgtgtaggcttagcctcaggtggcagatgtttgaggaaaggaattatgccaggaaggtgggaccgggttatggtcgggtttctattgggaatgctctttgtgcttttgggcatctgaatggaagctttacatagaaccttaggtagaactcccccaaatcgccatatttaaaaattattttcactctattcttgcttaaaactgtactcttttgcaaattaacaattttatcactgattcagagttaaaaagaagactaacttttcaagcaaatgcatctgtaaagatgctttagattagactgtcatgtctcagtgtctatctgtatatattatttgatattcagagaatctaaagcactcgtctactgttttaatgagatttaacagcttttaacagtgagtttcgtttgtaaactgcttgaagtctgtggcattcaggcacacgtctggctggccggctgggtctcctcccgggctcagtgggcctggggcctctctgacgtggtgcctgctggagggaggctcgtcgtcaccagctgactgctggtccggcttctgaccggcctttgtcctggctccgtagcagaacactgtaaaagtgcccgcgtctttgcagtagttgcagatttcagtcgtcgtgttacttgtgcacaaacagaagctgggtcttacccgcagcacgagtgtctcgggctgcccggagtcgcccgggagcaggtgctgcagccagagttacgcgggggccacgcgggccggcgggggtggggggaacgtgggggaacctgtgtttcacgtgactcagcagtgcccgccgccgtcaccagctatgcattcactccgtttccagtgagcagatgtcttgcttggaaagtggacctgtgtctgtgtctgtcctgagaacttaccagcagaaatcctcatttctgtgctacggatttaccaaaaattgtcaagtctttttcagtttaacagttcctttacatgtgtagtatttgaggaaaaaaatcaataaacagttgatctcgtgca

SEQ ID NO: 6 - DNA, 3693 bp, Mus musculus
  source 1..3693: mol_type = genomic DNA
gcggcgcggtcgcggctggcggcagaagcggctgcggcgggtctgggcagggccggggagctcggttggagcggaacgggaaggggtcggagtcgcagcccggccggggcggcgtgctcagtgggacccgaggcgaacggcggccgaggtctgtctgcaggttttacacatctgacccctgaactggagtgtctgctgccaccccctcgtcctctgcagaaatgtctgtcacctacgatgactctgtgggagtggaagtgtccagcgacagcttctgggaggttgggaactacaaacggactgtgaagcggattgacgatggccaccgcctgtgtggtgacctcatgaactgtctgcatgagcgggcacgcatcgagaaggcgtatgcacagcagctcactgagtgggcccgacgctggaggcagctggtagagaagggaccacagtatgggaccgtggagaaggcctggatagctgtcatgtctgaagcagagagggtgagtgaactgcacctggaagtgaaggcatcactgatgaatgaagactttgagaagatcaagaactggcagaaggaagcctttcacaagcagatgatgggaggcttcaaggagaccaaagaagcagaggatggctttcggaaggcccagaagccctgggccaagaagctgaaagaggtggaagcggcaaagaaggcgcaccacacagcgtgcaaagaggagaagctggccatctcccgggaagccaacagcaaggcagatccatccctcaaccctgagcagctgaagaaactgcaagacaagatagaaaaatgcaaacaggacgttctaaagaccaaggacaagtatgagaagtccctgaaggagcttgatcagaccacaccccagtacatggagaacatggagcaggtgttcgagcagtgccagcagtttgaagagaagcgcctgcgcttcttccgggaggttctgctggaggttcagaagcacttggatctgtccaatgtggctagctataaaaccatttaccgggagctggagcagagcatcaaagcagcagatgcggtagaggacctgaggtggttccgggctaaccatgggccaggcatggctatgaactggccacagtttgaggagtggtctgcagatctgaatcgaactctcagccggagagagaagaagaaggctgttgacggtgtcaccctaacagggatcaaccagacaggtgaccagtctggacagaacaagcctggcagcaaccttagtgtcccgagcaaccccgcccagtccacgcagttacagtccagctacaaccccttcgaggacgaggacgacacgggcagcagcatcagtgagaaggaggacattaaggccaaaaatgtcagcagctatgagaagactcagacttaccccactgactggtctgatgatgagtctaacaaccctttctcctccacggatgccaacggggattcgaacccatttgatgaggacacgacctcaggaacagaagtgcgagttcgggccctctatgactatgaggggcaggaacatgatgagctgagcttcaaggctggggatgaactgaccaagatagaggatgaagatgaacagggttggtgcaagggacgtttagacagcggccaggttggcctatacccagccaactatgtcgaggctatccagtgacagcccatgggcaggctggcggagagacggaaatgggcagttcaggagctccgttagccttggcctgggcagtgacacctctagtgcccccagcagccatgtaggcatccactccacctgcaaaagacgatggctctgttgttcttggcttcctggtgtgctttgaaggcagatgagctggtgatttcattgggcacttggcccttttccaagcacatctgggcagatatagacacaggaagatagggtccaacagcgagagccaggcccctccccacccccaccagctctctctatcatggatctgcaccttctcgcccttgtctctcctgagtcatgacgggtcatactgattcttgttccactgatgattttctctgatgaggtcctatctgcaaggtcaatgagcagacttacatgccatcttctgagtaaagagtttgaggttttaatttaaaggcaatgtacagctatacttttttatatgctcttccagtcagttaaattatggcctacactgatctgagatgttctccacgtgagctgtcttcatttctctgtgctatgtccagatgtggggttgctgcagccggggttctatggcaagtgccagttgcagggctaaccttgtgcaacgttccccaacacttccacatacagaaattattttcactctatccctgcttcagtttttgcagattaacagttctattagtgatttggaaagttaacagtaagaagactaacttttcaaaacagttgcatctgtagattaagatgctttacattagaccgttgtgtctcgatgtatatctgtatatattatttgataatcagaaaatctatagagttcacccactgttgaatgagagctggtggcttctgacagcagatctggtcaactgcttgaagcccatggcattgaagcacaggcacggctggttaacggtgcccacccagttaggatgtggctctggcctctgagtggagctgctgggaagactgattctcattggcctgggctccaagctcatgaccgagcactggaaaagctcctaggacttggtagtaatcgtagacttcacagtccctgtgtcactcactggagagctagagggaggggttcgacaccctccaccacacacacacacacacacacacacacacacacaagttcctccagttgcccttgtcctcaggtgcagtgggactgttgtgagccccagggatgggcacaaagaggacttttattttgttagctcggacagtgcagtggtgcacatcagcaacttgtatttcttcggtgtttggcacgagcactgtctcgctgtggctgtgtgtcatgagaacttaccagcagaaatccttgttcctaagctacagaatgaccaaaagctgtcaagtccttaatgtttagaaactccttaaaatgtatagtattttagaacaacaacaacaaaactcaataaacagttgatcttgtgtgtttgacagtccctttgccttgcatccaccctcatgttcacacagctgatagacctgtctccttgggggtgtaccagctttgaaactgggcaagatccagttcataggatcccttctcgggaccatgtctcagttcctgcaccagaggctggggacttggggcaccttgggcatccctgaggagggcatccctggtgccaccctctctcccctgaccaacaaccgctcctccccaaacctgcgtgtgttgacctttatgtgccactgttctgttttctcaagctgatgatctctgggtgtaatgtgatcaatgcaggtcccagctggtcctgctcgcacaaagaacctccaaggatcttgcctcctattgggatcagctaaggagataaagccagtgcggtagccctgaccagtcccggtgaacggactgctgctgctgctgctgctgctgctgctgctgctgctgctgctgctgctgctgctgctgctgctcttcaacactagtaaagcttctctctggccttcc

SEQ ID NO: 7 - protein, 486 aa, Homo sapiens
MSVTYDDSVGVEVSSDSFWEVGNYKRTVKRIDDGHRLCSDLMNCLHERARIEKAYAQQLTEWARRWRQLVEKGPQYGTVEKAWMAFMSEAERVSELHLEVKASLMNDDFEKIKNWQKEAFHKQMMGGFKETKEAEDGFRKAQKPWAKKLKEVEAAKKAHHAACKEEKLAISREANSKADPSLNPEQLKKLQDKIEKCKQDVLKTKEKYEKSLKELDQGTPQYMENMEQVFEQCQQFEEKRLRFFREVLLEVQKHLDLSNVAGYKAIYHDLEQSIRAADAVEDLRWFRANHGPGMAMNWPQFEEWSADLNRTLSRREKKKATDGVTLTGINQTGDQSLPSKPSSTLNVPSNPAQSAQSQSSYNPFEDEDDTGSTVSEKDDTKAKNVSSYEKTQSYPTDWSDDESNNPFSSTDANGDSNPFDDDATSGTEVRVRALYDYEGQEHDELSFKAGDELTKMEDEDEQGWCKGRLDNGQVGLYPANYVEAIQ

SEQ ID NO: 8 - protein, 486 aa, Mus musculus
MSVTYDDSVGVEVSSDSFWEVGNYKRTVKRIDDGHRLCGDLMNCLHERARIEKAYAQQLTEWARRWRQLVEKGPQYGTVEKAWIAVMSEAERVSELHLEVKASLMNEDFEKIKNWQKEAFHKQMMGGFKETKEAEDGFRKAQKPWAKKLKEVEAAKKAHHTACKEEKLAISREANSKADPSLNPEQLKKLQDKIEKCKQDVLKTKDKYEKSLKELDQTTPQYMENMEQVFEQCQQFEEKRLRFFREVLLEVQKHLDLSNVASYKTIYRELEQSIKAADAVEDLRWFRANHGPGMAMNWPQFEEWSADLNRTLSRREKKKAVDGVTLTGINQTGDQSGQNKPGSNLSVPSNPAQSTQLQSSYNPFEDEDDTGSSISEKEDIKAKNVSSYEKTQTYPTDWSDDESNNPFSSTDANGDSNPFDEDTTSGTEVRVRALYDYEGQEHDELSFKAGDELTKIEDEDEQGWCKGRLDSGQVGLYPANYVEAIQ

SEQ ID NO: 9 - DNA, 25 bp, synthetic construct
  source 1..25: mol_type = other DNA
ggtaagaatggcattaaatggatca

SEQ ID NO: 10 - DNA, 25 bp, synthetic construct
  source 1..25: mol_type = other DNA
gaatggcattaaatggatcacacct

SEQ ID NO: 11 - DNA, 25 bp, synthetic construct
  source 1..25: mol_type = other DNA
tggcattaaatggatcacaccttct

SEQ ID NO: 12 - DNA, 25 bp, synthetic construct
  source 1..25: mol_type = other DNA
ggcattaaatggatcacaccttctg

SEQ ID NO: 13 - DNA, 25 bp, synthetic construct
  source 1..25: mol_type = other DNA
cattaaatggatcacaccttctgca

SEQ ID NO: 14 - DNA, 25 bp, synthetic construct
  source 1..25: mol_type = other DNA
tcacaccttctgcagggatactcat

SEQ ID NO: 15 - DNA, 25 bp, synthetic construct
  source 1..25: mol_type = other DNA
acaccttctgcagggatactcatca

SEQ ID NO: 16 - DNA, 25 bp, synthetic construct
  source 1..25: mol_type = other DNA
caccttctgcagggatactcatcaa

SEQ ID NO: 17 - DNA, 25 bp, synthetic construct
  source 1..25: mol_type = other DNA
ccttctgcagggatactcatcaaag

SEQ ID NO: 18 - DNA, 25 bp, synthetic construct
  source 1..25: mol_type = other DNA
tctgcagggatactcatcaaagtca

SEQ ID NO: 19 - DNA, 21 bp, synthetic construct
  source 1..21: mol_type = other DNA
ggtaagaatggcattaaatgg

SEQ ID NO: 20 - DNA, 21 bp, synthetic construct
  source 1..21: mol_type = other DNA
ggcattaaatggatcacacct

SEQ ID NO: 21 - DNA, 21 bp, synthetic construct
  source 1..21: mol_type = other DNA
gcagggatactcatcaaagtc

SEQ ID NO: 22 - DNA, 25 bp, synthetic construct
  source 1..25: mol_type = other DNA
cacatatgatgattccgttggagta

SEQ ID NO: 23 - DNA, 25 bp, synthetic construct
  source 1..25: mol_type = other DNA
tcgggaactacaagcggactgtgaa

SEQ ID NO: 24 - DNA, 25 bp, synthetic construct
  source 1..25: mol_type = other DNA
caagaactggcagaaggaagccttt

SEQ ID NO: 25 - DNA, 25 bp, synthetic construct
  source 1..25: mol_type = other DNA
gaaagaggtagaagcagcaaagaaa

SEQ ID NO: 26 - DNA, 25 bp, synthetic construct
  source 1..25: mol_type = other DNA
caaagaggagaagctggctatctca

SEQ ID NO: 27 - DNA, 25 bp, synthetic construct
  source 1..25: mol_type = other DNA
caagcaagatgttcttaagaccaaa

SEQ ID NO: 28 - DNA, 25 bp, synthetic construct
  source 1..25: mol_type = other DNA
tagacctgtccaatgtggctggcta

SEQ ID NO: 29 - DNA, 25 bp, synthetic construct
  source 1..25: mol_type = other DNA
cctgtccaatgtggctggctacaaa

SEQ ID NO: 30 - DNA, 25 bp, synthetic construct
  source 1..25: mol_type = other DNA
cagctacgagaagacccagagctat

SEQ ID NO: 31 - DNA, 25 bp, synthetic construct
  source 1..25: mol_type = other DNA
agttggcctatacccggcaaattat

SEQ ID NO: 32 - RNA, 19 nt, synthetic construct
  source 1..19: mol_type = other RNA
caaattatgtggaggcgat

SEQ ID NO: 33 - RNA, 19 nt, synthetic construct
  source 1..19: mol_type = other RNA
cccttaatgtcccgagcaa

SEQ ID NO: 34 - RNA, 19 nt, synthetic construct
  source 1..19: mol_type = other RNA
cctcactgatgaacgatga

SEQ ID NO: 35 - RNA, 19 nt, synthetic construct
  source 1..19: mol_type = other RNA
ctgaggtggttccgagcca

SEQ ID NO: 36 - DNA, 21 bp, synthetic construct
  source 1..21: mol_type = other DNA
ggcctcactgatgaacgatga

SEQ ID NO: 37 - DNA, 21 bp, synthetic construct
  source 1..21: mol_type = other DNA
ggtagaagcagcaaagaaagc

SEQ ID NO: 38 - DNA, 21 bp, synthetic construct
  source 1..21: mol_type = other DNA
gcaagcaagatgttcttaaga

SEQ ID NO: 39 - DNA, 21 bp, synthetic construct
  source 1..21: mol_type = other DNA
gcacctagacctgtccaatgt

SEQ ID NO: 40 - DNA, 21 bp, synthetic construct
  source 1..21: mol_type = other DNA
ggctggctacaaagccattta

SEQ ID NO: 41 - DNA, 21 bp, synthetic construct
  source 1..21: mol_type = other DNA
gctggctacaaagccatttac

SEQ ID NO: 42 - DNA, 21 bp, synthetic construct
  source 1..21: mol_type = other DNA
gctacaaagccatttaccatg

SEQ ID NO: 43 - DNA, 21 bp, synthetic construct
  source 1..21: mol_type = other DNA
ggtcagacgatgagtctaaca

SEQ ID NO: 44 - DNA, 21 bp, synthetic construct
  source 1..21: mol_type = other DNA
ggcctatacccggcaaattat

SEQ ID NO: 45 - DNA, 21 bp, synthetic construct
  source 1..21: mol_type = other DNA
gcctatacccggcaaattatg

(19) United States
(12) Patent Application Publication
(10) Pub. No.: US 2026/0347841 A1
(43) Pub. Date: Nov. 5, 2026

(54) REGULATION OF BLOOD-CENTRAL NERVOUS SYSTEM (BLOOD-CNS) BARRIER AND USES THEREOF

(71) Applicant: President and Fellows of Harvard College, Cambridge, MA (US)

(72) Inventors: Chenghua Gu, Cambrdge, MA (US); Shachar Y. Dagan, Cambrdge, MA (US)

(73) Assignee: President and Fellows of Harvard College, Cambridge, MA (US)

(21) Appl. No.: 19/111,711

(22) PCT Filed: Sep. 14, 2023
(86) PCT No.: PCT/US2023/074158

§ 371 Date: Mar. 13, 2025

Related U.S. Application Data
(60) Provisional application No. 63/406,463, filed on Sep. 14, 2022.

(51) Int. Cl. C12N 15/113; A61K 31/7088; A61K 45/06; A61K 48/00; A61P 25/00; C07K 14/47; C12N 15/86

(52) U.S. Cl. CPC C12N 15/113; A61K 31/7088; A61K 45/06; A61K 48/005; A61P 25/00; C07K 14/4702; C12N 15/86; C12N 2310/14; C12N 2310/531; C12N 2320/32; C12N 2750/14143

(57) ABSTRACT

The present disclosure provides compositions, methods, kits and uses for regulating blood-Central Nervous System (blood-CNS) barrier permeability (e.g., increasing or decreasing blood-CNS barrier permeability) by regulating signaling between endothelial cell derived Cede 141 and Pacsin2 expressed in CNS endothelial cells.

### RELATED APPLICATIONS

This application claims priority under 35 U.S.C. § 119 (e) to U.S. Provisional Application, U.S. Ser. No. 63/406,463, filed Sep. 14, 2022, which is incorporated herein by reference.

### GOVERNMENT SUPPORT

This invention was made with government support under grant numbers NS116820 and HL153261 awarded by the National Institutes of Health (NIH). The government has certain rights in the invention.

### REFERENCE TO AN ELECTRONIC SEQUENCE LISTING

The contents of the electronic sequence listing (H082470416WO00-SEQ-LJG.xml; Size: 64,042 bytes; and Date of Creation: Sep. 11, 2023) is incorporated herein by reference in its entirety.

### BACKGROUND

The CNS requires an optimal and tightly regulated microenvironment for efficient synaptic transmission. This is achieved by blood-CNS barriers that regulate substance flux to maintain tissue homeostasis. Barrier properties of CNS endothelial cells require induction and maintenance from brain parenchymal cells. However, the signal(s) that facilitate endothelial cells to maintain barrier integrity remain elusive.

### SUMMARY

The present disclosure, at least in part, provides compositions and the uses thereof, kits and methods for regulating Blood-Central Nervous System (blood-CNS) barrier permeability (e.g., increasing or decreasing Blood-CNS barrier permeability) by regulating signaling between Ccdc141 and Pacsin2. In some embodiments, the methods described herein comprises regulating (e.g., increase) blood-CNS barrier permeability by administering to a subject an inhibitor of Ccdc141, and/or an agent that increases Pacsin2 expression and/or activity. In some embodiments, the methods described herein comprises regulating (e.g., decrease) blood-CNS barrier permeability by administering to a subject an inhibitor of Pacsin2, and/or an agent that increases Ccdc141 expression and/or activity.

According to some aspects, a method for increasing Blood-Central Nervous System (Blood-CNS) Barrier permeability to treat a CNS disease in a subject is provided herein. In some embodiments, the method comprises administering the subject an inhibitor of Ccdc141 at the Blood-CNS Barrier.

In some embodiments, the blood-CNS barrier is the blood-brain barrier. In other embodiments, the blood-CNS barrier is the blood-retina barrier.

In some embodiments, the inhibitor of Ccdc141 is capable of inhibiting Ccdc141 expression and/or activity. In some embodiments, the inhibitor of Ccdc141 is an inhibitory nucleic acid targeting Ccdc141. In some embodiments, the inhibitory nucleic acid targets the coding sequence of Ccdc141 comprising SEQ ID NO: 1 or SEQ ID NO: 2. In some embodiments, the inhibitory nucleic acid targeting Ccdc141 is a siRNA, a shRNA, a miRNA, an AmiRNA, an aptamer, or an ASO.

In some embodiments, the inhibitory nucleic acid targeting Ccdc141 is a siRNA targeting Ccdc141. In some embodiments, the siRNA targeting Ccdc141 comprises an antisense strand complementary to any one of SEQ ID NOs: 9-21. I

In some embodiments, the inhibitory nucleic acid targeting Ccdc141 is a shRNA targeting Ccdc141. In some embodiments, the shRNA comprises a guide strand complementary to any one of SEQ ID NOs: 9-21.

In some embodiments, the inhibitor of Ccdc141 is an antibody, an antibody variant or an antigen-binding fragment targeting Ccdc141.

In some embodiments, the inhibitor of Ccdc141 is a small molecule.

In some embodiments, the inhibitor of Ccdc141 is delivered to CNS endothelial cells.

In some embodiments, the injection is systemic injection comprising intravenous injection, intramuscular injection, subcutaneous injection, or intraperitoneal injection.

In some embodiment, the injection is direct injection to the CNS comprising intracerebral injection, intraventricular injection, intracisternal injection, intraparenchymal injection, intrathecal injection, and any combination thereof.

According to some aspects, the present disclosure provides a method for increasing Blood-Central Nervous System (Blood-CNS) Barrier permeability to treat a CNS disease in a subject is provided herein. In some embodiments, the method comprising administering an isolated nucleic acid comprising a transgene encoding Pacsin2.

In some embodiments, the inhibitory nucleic acid is delivered by a recombinant adeno-associated virus (rAAV). In some embodiments, the capsid protein is BR1, BI30, AAV1, AAV2, AAV9, or variant thereof (e.g., AAV-PHP or AAV-PHP.B). In some embodiments, the capsid protein is BR1. In some embodiments, the capsid protein is BI30. In some embodiments, the inhibitor of Ccdc141 is administered to the subject via injection.

In some embodiment, the transgene further comprises a promoter operably linked to a nucleotide sequence encoding Pacsin2. In some embodiments, the isolated nucleic acid further comprises two adeno-associated virus inverted terminal repeats (ITRs) flanking the transgene encoding Pacsin2. In some embodiments, the isolated nucleic acid is administered to the subject via a recombinant adeno-associated virus (rAAV). In some embodiments, the rAAV further comprises a capsid protein. In some embodiments, the rAAV comprises a capsid protein comprising BR1, BI30, AAV1, AAV2, AAV9, or variant thereof. In some embodiments, the capsid protein BR1. In some embodiments, the capsid protein is BI30.

In some embodiments, the method further comprises administering a therapeutic agent for treating the CNS disease.

According to some aspects, the present disclosure provides a method for delivering a therapeutic agent to the central nervous system to treat a CNS disease is described herein. In some embodiments, the method comprises administering a subject, a therapeutic agent for treating the disease; and an agent for increasing Blood-Central Nervous System (Blood-CNS) Barrier.

In some embodiments, the agent for increasing Blood-CNS barrier permeability is an inhibitor of Ccdc141.

In some embodiments, the agent for increasing Blood-CNS barrier permeability is an isolated nucleic acid comprising a transgene encoding Pacsin2.

In some embodiments, the CNS disease is a neuromuscular disease, neurodegenerative disease, brain and nerve tumors, Neurogenetic Diseases, Cognitive disorders, Familial dystonia, Neuroinfectious disease, neuropsychiatric disorders. In some embodiments, the neuromuscular disease is Amyotrophic Lateral Sclerosis (ALS), Ataxia, Cerebral Palsy, Muscular Dystrophy.

In some embodiments, the therapeutic agent comprises antibiotics, antibodies, anticonvulsants (e.g., gabapentin), chemotherapeutics, anti-inflammatoires, neurotransmitters, pain medication (e.g., morphine), peptides, nucleic acids (e.g. RNAi-based therapies), or psychiatric drugs.

According to some aspects, the present disclosure provides a method for decreasing Blood-Central Nervous System (Blood-CNS) Barrier permeability to treat a CNS disease in a subject. In some embodiments, the method comprises administering the subject an inhibitor of Pacsin2 at the Blood-CNS Barrier.

In some embodiments, the inhibitor of Pacsin2 is capable of inhibiting Pacsin2 expression and/or activity. In some embodiments, the inhibitor of Pacsin2 is an inhibitory nucleic acid targeting Pacsin2. In some embodiments, the inhibitory nucleic acid targets the coding sequence of Pacsin2 comprising SEQ ID NOs: 5 and 6. In some embodiments, the inhibitory nucleic acid targeting Pacsin2 is a siRNA, a shRNA, a miRNA, an AmiRNA, an aptamer, or an ASO. In some embodiments, the inhibitory nucleic acid targeting Pacsin2 is a siRNA targeting Pacsin2. In some embodiments, the siRNA targeting Pacsin2 comprises an antisense strand complementary to any one of SEQ ID NOs: 22-31, and 36-45.

In some embodiments, the inhibitory nucleic acid targeting Pacsin2 is a shRNA targeting Pacsin2. In some embodiments, the shRNA targeting Pacsin2 comprises a guide strand complementary to any one of SEQ ID NOs: 22-31, and 36-45.

In some embodiments, the inhibitor of Pacsin2 is an antibody, an antibody variant or an antigen-binding fragment targeting Pacsin2.

In some embodiments, the inhibitor of Pacsin2 is a small molecule.

In some embodiments, the inhibitor of Pacsin2 is delivered to CNS endothelial cells.

In some embodiments, the inhibitor of Pacsin2 is delivered by a recombinant adeno-associated virus (rAAV). In some embodiments, the rAAV comprises a capsid protein comprising BR1, BI30, AAV1, AAV2, AAV9, or variant thereof. In some embodiments, the capsid protein is BR1. In some embodiments, the capsid protein is BI30.

In some embodiments, the inhibitor of Pacsin2 is administered to the subject via injection. In some embodiments, the injection is systemic injection comprising intravenous injection, intramuscular injection, subcutaneous injection, or intraperitoneal injection. In some embodiments, the injection is direct injection to the CNS comprising intracerebral injection, intraventricular injection, intracisternal injection, intraparenchymal injection, intrathecal injection, intravitreal injection, and any combination thereof.

According to some aspects, the present disclosure provides methods for decreasing Blood-Central Nervous System (Blood-CNS) Barrier permeability to treat a CNS disease in a subject are provided herein. In some embodiments, the method comprises administering an isolated nucleic acid comprising a transgene encoding Ccdc141.

In some embodiments, the transgene further comprises a promoter operably linked to a nucleotide sequence encoding Ccdc141.

In some embodiments, the isolated nucleic acid further comprises two adeno-associated virus inverted terminal repeats (ITRs) flanking the transgene encoding Ccdc141. In some embodiments, the isolated nucleic acid is administered to the subject via a recombinant adeno-associated virus (rAAV). In some embodiments, the rAAV further comprises a capsid protein. In some embodiments, the capsid protein is BR1, BI30, AAV1, AAV2, AAV9, or variant thereof. In some embodiments, the capsid protein is BR1. In some embodiments, the capsid protein is BI30.

In some embodiments, the CNS disease is retinal disease, neurodegenerative disease, acute injury of the CNS, neuroinfectious disease, primary and metastatic cancers f the CNS, autoimmune disease of the CNS, neuroinflammatory conditions, or cognitive disorder. In some embodiments, the retinal disease is diabetic retinopathy. In some embodiments, the neurodegenerative disease is Huntington's disease, Alzheimer's disease, Parkinson's disease, motor neuron disease, Amyotrophic lateral sclerosis, spinal muscular atrophy, spinocerebral ataxia. In some embodiments, the acute injury of the CNS is stroke or head trauma. In some embodiments, the neuroinfectious disease is encephalitis, sepsis, or COVID-19. In some embodiments, the primary cancer of the CNS is glioblastoma, meningioma, or lymphoma. In some embodiments, the metastatic cancer of the CNS is lung cancer, metastatic breast cancer, GI track cancers, or melanoma. In some embodiments, the autoimmune disease of the CNS is multiple sclerosis. In some embodiments, the neuroinflammatory condition is CNS Lupus, CNS Lyme Disease, Neurosarcoidosis, Neuromyelitis optica (NMO), or Paraneoplastic and Autoimmune Encephalitis.

In some embodiments, the cognitive disorder is dementia resulting from Alzheimer's disease, Lewy body dementia, frontotemporal dementia, encephalopathy, or post-acute COVID syndrome.

In some aspects, the present disclosure provides composition and method for treating a condition associated with increased blood-CNS barrier permeability, the method comprising administering to a subject a Pacsin2 inhibitor or an agent promoting Ccdc141 expression/activity. In some embodiments, the condition is associated with aging. In some embodiments, the condition is early onset dementia. In some embodiments, the condition is stroke. In some embodiments, the condition is spinal cord injury.

The details of one or more embodiments of the invention are set forth in the description below. Other features or advantages of the present invention will be apparent from the following drawing and detailed description of certain embodiments and also from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the Detailed Description of Specific Embodiments presented herein.

FIGS. 1A-1H show the delivery of gene-specific single-guide RNA (sgRNAs) using adeno-associated virus (AAV) that specifically infects brain microvessels called AAV-BR1 and results in increased blood-brain barrier leakage. FIG. 1A shows a genetic construct containing sgRNA incorporated into AAV-BR1 and injected into mice to achieve CNS-specific CRISPR/Cas9 gene knockout. FIG. 1B shows expression of AAV-BR1 (left panel) in CNS endothelial cells (right paten). FIG. 1C shows the elimination of Glut1 expression in CNS endothelial cells infected with AAV-BR1 sgRNA: Slcla. FIG. 1D shows that elimination of Glut1 through AAV-Glut1 injection results in blood-brain barrier leakage.

FIG. 1E shows increased brain leakage in the brain of mice injected with BR1-sgRNA:Slc2a1 relative to controls. FIG. 1F shows increased brain leakage in coronal sections of a mouse brain injected with BR1-sgRNA:Slc2a1 relative to controls. FIG. 1G shows increased leakage area outside of Glut1^{KD} vessels in the brains of mice injected with BR1-sgRNA:Slc2a1 relative to controls. FIG. 1H shows quantifications of increased tracer leakage outside of vessels in Glut1^{KD} vs controls.

FIGS. 2A-2D show the enrichment of Ccdc141 to the vessels of the CNS. FIG. 2A shows the vessels, nuclei and, Ccdc141 mRNA 1 in the brain and lungs. FIG. 2B shows the mRNA density of Ccdc141 (dots/area) in the brain and lungs. FIG. 2C shows Ccdc141 protein expression in purified endothelial prep from brain and lung. FIG. 2D shows Ccdc141 protein quantification using western-blot of purified endothelial prep from brain and lung. This analysis showed 4-fold higher Ccdc141 protein in brain ECs compared to lung ECs.

FIGS. 3A-3G show effects of knocking down Ccdc141 expression in CNS endothelial cells. FIG. 3A shows Ccdc141 ablation using AAV-BR1 delivered sgRNAs targeting Ccdc141. FIG. 3B shows no apparent tracer extravasation in the brains of control mice. FIG. 3C shows increased tracer extravasation in the brains of Cdc141^{KD} mice. FIG. 3D shows increased leakage area outside of Ccdc141^{KD} vessels in the brain of mice injected with BR1-sgRNA: Ccdc141 relative to controls. FIG. 3E shows the correlation of virus infection in vessels to leakage sites as measured by increased NHS-biotin concentrations surrounding vessels in sgCcdc141 injected mice versus controls. FIG. 3F shows quantifications of increased tracer leakage outside of vessels in Ccdc141^{KD} vs controls. FIG. 3G Shows increased endogenous IgG leakage outside of Ccdc141KD vessels in the brain of mice injected with BR1-sgRNA:Slc2a1 relative to controls.

FIGS. 4A-4B show the effects of endothelial-specific Ccdc141^{KD} on endothelial cells and neurons of the brain. FIG. 4A shows that acute endothelial specific ablation of Ccdc141 results in neuronal cell death. FIG. 4B shows that acute endothelial specific ablation of Ccdc141 does not affect endothelial cell viability.

FIGS. 5A-5B show the effects of Ccdc141^{KD} on vessel area in the CNS. FIG. 5A shows the percent vessel area in the CNS in Ccdc141^{KD} and control mice. FIG. 5B shows confocal micrographs of control and Ccdc141^{KD} mice.

FIGS. 6A-6E show the effects of Ccdc141 on BBB function through suppression of tubular vesicle trafficking. FIG. 6A shows an increase in intracellular tubular transport vesicles in Ccdc141^{KD} CNS endothelial cells vs controls. FIG. 6B shows increases in total vesicle density and tubular vesicle density in Ccdc141^{KD} mice relative to control mice. There was no change in round vesicle density between Ccdc141^{KD} mice and control mice. FIG. 6C shows magnified electron microscopy images showing the tubular structure of the transport vesicles following Ccdc141 knockdown. FIG. 6D shows that Ccdc141^{KD} has no effect on caveolae-mediated transcytosis at the BBB in CNS endothelial cells. FIG. 6E shows the quantification of Cav1 vesicle density in Ccdc141^{KD} and control mouse brains.

FIGS. 7A-7D show the effects of Ccdc141^{KD} on tight-junctions in the BBB endothelial cells of the CNS. FIG. 7A shows that Ccdc141^{KD} does not induce tracer transfer between adjacent endothelial cells, therefore exhibiting functional tight junctions. FIG. 7B shows that Wnt/β-catenin signaling is unchanged after Ccdc141 ablation in brain endothelial cells. FIG. 7C shows that Cldn5 expression in CNS endothelial cells was unaffected following Ccdc141^{KD} relative to controls. FIG. 7D shows that the expression of tight-junction protein ZO-1 was unaffected following Ccdc141^{KD} relative to controls.

FIGS. 8A-8F show that Pacsin2 expression is increased following Ccdc141^{KD}. FIG. 8A shows the relative expression of Pacsin2 mRNA in the brain and lungs of mice. FIG. 8B shows Pacsin2 protein expression in purified endothelial prep from brain and lung FIG. 8C shows Pacsin2 protein quantification using western blot of purified endothelial prep from brain and lung. FIG. 8D shows increased Pacsin2 expression in Ccdc141^{KD} vessels relative to controls. FIG. 8E shows a graph demonstrating the increased percentage of Pacsin2 mRNA positive vessels following Ccdc141^{KD} relative to controls. FIG. 8F shows that ablation of Ccdc141 in CNS capillaries results in upregulation of Pacsin2.

FIGS. 9A-9J show that Pacsin2 overexpression in CNS endothelial cells increases BBB leakage and tubular vesicle transcytosis. FIG. 9A shows increased Pacsin2 expression in brain endothelial cells following injection with AAV-BR1 expressing Pacsin2 in adult mice. FIG. 9B shows a graph demonstrating the increased percentage of Pacsin2 positive vessels following Pacsin2 overexpression relative to controls. FIG. 9C shows Pacsin2 protein upregulation following Pacsin2 overexpression compared to controls. FIG. 9D shows that Pacsin2 overexpression increases brain leakiness as measured by increased permeation of sulfo-NHS-biotin into adult mouse brain tissues. FIG. 9E shows confocal micrographs of leakage of sulfo-NHS-biotin into brain regions surrounding the vessels of adult mice overexpressing Pacsin2. FIG. 9F shows a graph quantifying the leakage of sulfo-NHS-biotin into adult mouse brain tissues surrounding vessels of endothelial cells overexpressing Pacsin2. FIG. 9G contains electron microscopy images showing that Pacsin2 overexpression results in increased tubular vesicles in CNS endothelial cells relative to controls. FIG. 9H shows increase in tubular vesicle density following Pacsin2 overexpression relative to controls. FIG. 9I shows that Pacsin2 overexpression results in the leakage of mouse endogenous IgG (mIgG). FIG. 9J shows a graph quantifying the leakage of endogenous mouse IgG into adult mouse brain parenchyma.

FIGS. 10A-10B show that Ccdc141 suppressing tubular vesicle mediated transcytosis and BBB leakiness by inhibiting Pacsin2 expression. FIG. 10A shows that double knock-down of Pacsin2 and Ccdc141 in adult mouse brains rescues leakiness phenotype observed in Ccdc141^{KD} animals. FIG. 10B shows a graph quantifying the percentage of leakage area outside of the vessels in Cdc141^{KD}, Pacsin2^{KD}, and Ccdc141^{KD}+Pacsin^{KD} adult mouse brains.

FIG. 11 shows Pacsin2 protein (middle panel) in the vessels (represented by Glut1 expression in the left panel) of the post-mortem temporal cortex of the aged human individual (78 years old). The right panel shows a merged image of Glut1 and Pacsin2 expression.

FIG. 12 shows that Pacsin2 is up-regulated in the endothelial cells in HSV-induced neuroinflammation. The immunostaining of mouse brain stem sections shows increased Pacsin2 protein levels (middle panel in infected region compared to middle panel in uninfected region) in the vessels (represented by Icam2 expression in the left panel of the uninfected region and the infected region) of the HSV infected mice compared to uninfected mice.

### DETAILED DESCRIPTION

The present disclosure, at least in part, provides compositions and uses thereof, kits and uses thereof, and methods for regulating Blood-Central Nervous System (blood-CNS) barrier permeability (e.g., increasing or decreasing blood-CNS barrier permeability) by regulating the Coiled-Coil Domain Containing 141 (Ccdc141). In some aspects, the present disclosure provides compositions and uses thereof, kits and uses thereof, and methods for regulating Blood-Central Nervous System (blood-CNS) barrier permeability (e.g., increasing or decreasing blood-CNS barrier permeability) by regulating protein kinase C and casein kinase substrate in neurons protein 2 (Pacsin2). In some embodiments, the present disclosre provides composition and methods for regulating Blood-Central Nervous System (blood-CNS) barrier permeability (e.g., increasing or decreasing blood-CNS barrier permeability) by regulating Ccdc141 and Pacsin2.

Ccdc141 is linked to congenital hypogonadotropic hypogonadism in humans. During embryonic development, gonadotropin releasing hormone (GnRH) neurons originating in the nasal placode migrate into the dorsal region of the olfactory bulb (OB) and then caudally towards the hypothalamus (Hutchins et al., CCDCl41 mutation identified in anosmic hypogonadotropic hypogonadism (Kallmann syndrome) alters GnRH neuronal migration. Endocrinology, 157 (5), 1956-1966 (2016)) Genetic screenings identified families with congenital hypogonadotropic hypogonadism carry novel mutations in Ccdc141 and hypothesized a role in neuronal migration. The present disclosure, at least in part, is based on the discovery that Ccdc141 is expressed by CNS endothelial cells. In some embodiments, Ccdc141 plays a role in regulating CNS endothelial cell function (e.g., regulating blood-CNS barrier permeability). In some embodiments, Ccdc141 regulates blood-CNS barrier permeability via Pacsin2. In some embodiments, the presence of Ccdc141 in CNS endothelial cells inhibits Pacsin2. In some embodiments, inhibition of Pacsin2 by Ccdc141 results in decreased tubular-vesicle mediated transcytosis of the CNS endothelial cells.

Pacsin2 colocalizes with amyloid-β particles, suggesting that Pacsin2 may have a protective role in the aging brain by providing clearance of amyloid-β particles. Pacsin2 is normally expressed at low levels in brain endothelial cells (ECs) compared to periphery ECs but is upregulated in EC-specific knockdown of Ccdc141 mutant mice. In some embodiments, overexpression of Pacsin2 in brain ECs increases blood-CNS barrier permeability. In some embodiments, overexpression of Pacsin2 in brain ECs up-regulates tubular vesicles. In some embodiments, knocking down Pacsin2 in brain ECs decreases blood-CNS barrier permeability (e.g., rescued the leakage phenotype observed in brain EC-specific Ccdc141 knockdown). The present disclosure enables specific manipulation of blood-CNS barrier permeability to improve the delivery of therapeutics, or to ameliorate ailments associated with blood-CNS leakage.

Ccdc141 inhibits tubular vesicle transcytosis that is mediated by Pacsin2. Ccdc141 gene ablation or Pacsin2 gene overexpression in brain endothelial cells in mice leads to an accumulation of tracer-filled tubular vesicles (e.g., tubular vesicles are involved in transcytosis (i.e., vesicular trafficking) of molecules across the blood-brain barrier endothelial cell layer, from blood into the brain tissue). Analogous tracer-filled tubular vesicles have been observed in the disrupted blood-brain barrier vasculature in the brain and spinal cord, under several neuropathological conditions. Specifically, tubular vesicles have been observed in the brain endothelial cells after traumatic brain injuries suggesting Pacsin2 overexpression in these conditions (see, e.g., Lossinsky et al., New ultrastructural evidence for a protein transport system in endothelial cells of gerbil brains. Acta Neuropathol. 47, 105-110; Lossinsky et al., Ultracytochemical studies of vesicular and canalicular transport structures in the injured mammalian blood-brain barrier. Acta Neuropathol. 61, 239-245; Lossinsky et al., Ultracytochemical evidence for endothelial channel-lysosome connections in mouse brain following blood-brain barrier changes. Acta Neuropathol. 53, 197-202; Lossinsky et al., A comparative ultrastructural study of endothelial cell tubular structures from injured mouse blood-brain barrier and normal hepatic sinusoids demonstrated after perfusion fixation with osmium tetroxide. Microvasc Res 31, 333-344) and stroke (see, e.g., Tagami, M. et al., Increased transendothelial channel transport of cerebral capillary endothelium in stroke-prone SHR. Stroke 14, 591-596) and in the spinal cord endothelial cells in the experimental autoimmune encephalomyelitis (EAE) mouse and rat models for multiple sclerosis (see, e.g., Claudio et al., Increased vesicular transport and decreased mitochondrial content in blood-brain barrier endothelial cells during experimental autoimmune encephalomyelitis. Am J Pathol 135, 1157-1168 (1989); Lossinsky et al., Sites of egress of inflammatory cells and horseradish peroxidase transport across the blood-brain barrier in a murine model of chronic relapsing experimental allergic encephalomyelitis. Acta Neuropathol 78, 359-371. In other embodiments, Pacsin2 is overexpressed in ageing brain where blood-CNS barrier permeability has been shown to be increased (e.g., early onset dementia such as frontotemporal dementia (see, Gerrits et al., Neurovascular dysfunction in GRN-associated frontotemporal dementia identified by single-nucleus RNA sequencing of human cerebral cortex, Nat. Neurosci. 2022 August; 25(8): 1034-1048). In some embodiments, Ccdc141 is decreased in conditions where blood-CNS barrier permeability have been shown to be increased (e.g., stoke (see, Garcia-Bonilla et al., Brain and blood single-cell transcriptomics in acute and subacute phases after experimental stroke; bioRxiv. Preprint. 2023 Apr. 3), spinal cord injury (see, Cao et al., Single-cell RNA sequencing for traumatic spinal cord injury, The FASEB Journal. 2022; 36:e22656), ageing (see, Ximerakis et al., Single-cell transcriptomic profiling of the aging mouse brain, Nature Neuroscience, volume 22, pages 1696-1708 (2019)). Accordingly, in some embodiments, the present disclosure contemplates treating diseases and conditions associated with increased blood-CNS permeability (e.g., ageing, early onset dementia, stroke, or traumatic spinal cord injury, Multiple sclerosis, epilepsy, cerebral edema) by administering a subject in need thereof an agent that inhibits Pacsin2 expression/activity and/or promotes Ccdc141 expression/activity. In some embodiments, the present disclosure provides methods of inhibiting transcytosis at the blood-CNS barrier by administering to a subject in need thereof an agent that inhibits Pacsin2 expression/activity and/or promotes Ccdc141 expression/activity. The blood-CNS barrier is a structure that separates circulating blood from the central nervous system (CNS). In some embodiments, the blood-CNS barrier includes the blood-brain barrier (BBB) and blood-retina barrier (BRB). The blood-CNS barrier lines the capillaries associated with the CNS and is comprised of endothelial cells and the tight junctions between them. The blood-brain barrier is formed by endothelial cells of the blood vessel (e.g., capillary) wall, astrocyte end-feet ensheathing the capillary, and pericytes embedded in the blood vessel (e.g., capillary) basement membrane. In some embodiments, the endothelial cells of the CNS are brain endothelial cells. In some embodiments, the brain endothelial cells are arterial endothelial cells, venous endothelial cells, and/or capillary endothelial cells. In some embodiments, the brain capillary endothelial cells are brain microvascular endothelial cells (BMVECs). In some embodiments, the endothelial cells of the CNS are spinal cord endothelial cells. In some embodiments, the endothelial cells of the CNS are retina vasculature endothelial cells. In some embodiments, the retina vasculature endothelial cells are superficial plexus arterial endothelial cells, superficial plexus venous endothelial cells, intermediate plexus endothelial cells, or deep plexus endothelial cells.

The blood-CNS barrier generally excludes large hydrophilic molecules and pathogens (e.g., bacteria, viruses, or parasites) from entering the CNS while allowing the passage of small hydrophobic molecules, such as lipids and oxygen. Other molecules are actively transported across the blood-CNS barrier, e.g., glucose. The restrictive permeability of CNS endothelial cells that constitute these barriers is a result of specialized tight junctions and low rates of transcytosis, which limit substance exchange between blood and CNS tissue. While the blood-CNS barrier is generally very effective at excluding, e.g., pathogens, from the CNS, the blood-CNS barrier poses a formidable obstacle when a drug needs to be delivered to the CNS. For example, antibodies and most antibiotics will not cross the blood-CNS barrier. The degradation of the blood-CNS barrier is a feature of many neurodegenerative diseases, e.g., multiple sclerosis. Accordingly, methods and compositions for modulating the permeability of the blood-CNS barrier, both by increasing or decreasing the permeability of the barrier, have a role in the treatment of a wide variety of diseases that impact the CNS. (See, e.g., Kadry et al., A blood-brain barrier overview on structure, function, impairment, and biomarkers of integrity, Fluids and Barriers of the CNS, volume 17, Article number: 69 (2020)).

The present disclosure, at least in part, is based on the theory that the barrier properties of the CNS endothelial cells are not intrinsic to those cells. Rather, the CNS endothelial cells require active induction and maintenance from the CNS environment to induce and maintain the barrier properties (see, e.g., P. A. Stewart et al., Developing nervous tissue induces formation of blood-brain barrier characteristics in invading endothelial cells: A study using quail-chick transplantation chimeras. Dev. Biol. 84, 183-192 (1981)). In some aspects, the present disclosure is based on the discovery that the scaffolding protein coiled-coil domain 141 (Ccdc141) inhibits expression of protein kinase C and casein kinase substrate in neurons 2 (Pacsin2) protein in CNS endothelial cells. In some embodiments, Ccdc141 is expressed by CNS endothelial cells. In some embodiments, the Ccdc141 protein expressed by CNS endothelial cells regulates tubular vesicle-mediated transcytosis via inhibition of Pacsin2 in endothelial cells in the CNS. In some embodiments, inhibition of Ccdc141 increases Pacsin2 expression in CNS endothelial cells and increases blood-CNS barrier permeability by increasing transcytosis in CNS endothelial cells. In some embodiments, inhibition of Pacsin2 in CNS endothelial cells decreases blood-CNS barrier permeability by regulating transcytosis (e.g., tubular vesicle mediated transcytosis) in CNS endothelial cells.

In some aspects, the present disclosure provides compositions and methods for increasing Blood-Central Nervous System (blood-CNS) barrier permeability to treat a disease (e.g., brain and CNS tumors, ALS, etc.) in a subject, the method comprising administering to the subject an inhibitor (e.g., inhibitory nucleic acids, small molecule inhibitors, and/or antibodies targeting Ccdc141) of Ccdc141 at the blood-CNS barrier. In some aspects, the present disclosure also provides methods for increasing blood-CNS barrier permeability, the method comprising administering to a subject an activator of Pacsin2 (e.g., coding sequence of Pacsin2). In some aspects, the present disclosure provides compositions and methods for decreasing blood-CNS barrier permeability for treating or preventing diseases associated with increased blood-CNS barrier permeability (e.g., ageing related neurodegenerative conditions (e.g., early onset dementia), brain vasculature diseases (e.g., stroke, spinal cord injury), chemical exposures, and/or bacterial and viral infections) in a subject, the method comprising administering to a subject an inhibitor (e.g., inhibitory nucleic acids, small molecule inhibitors, and/or antibodies targeting Pacsin2) of Pacsin2 signaling. In some aspects, the present disclosure also provides compositions and methods of decreasing blood-CNS permeability for treating or preventing diseases associated with increased blood-CNS barrier permeability (e.g., ageing related neurodegenerative conditions (e.g., early onset dementia), brain vasculature diseases (e.g., stroke, spinal cord injury), chemical exposures, and/or bacterial and viral infections) in a subject, the method comprising increasing Ccdc141 expression and/or activity in the CNS endothelial cells (e.g., overexpressing Ccdc141).

Other aspects of the present disclosure relate to co-delivering a molecule (e.g., a therapeutic agent) with an inhibitor of Ccdc141 or an activator of Pacsin2 (e.g., Pacsin2 coding sequence) at the blood-CNS Barrier to facilitate the CNS delivery of the molecule (e.g., a therapeutic agent).

In some aspects, the present disclosure provides methods for decreasing Blood-Central Nervous System (blood-CNS) barrier permeability for treating a disease in a subject, the method comprising administering to the subject an agent that inhibits Pacsin2 or increases Ccdc141 expression and/or activity in the central nervous system (CNS).

I. Increasing Blood-Central Nervous System (blood-CNS) Barrier Permeability

In some aspects, the present disclosure provides compositions and methods, kits and uses for increasing blood-CNS barrier permeability. In some embodiments, the method comprises administering to a subject an inhibitor of Ccdc141 signaling. In some embodiments, the Ccdc141 is expressed by CNS endothelial cells. In some embodiments, the method comprises administering to the subject an agent that promotes Pacsin2 expression and/or activity (e.g., an isolated nucleic acid encoding Pacsin2) at the blood-CNS barrier.

In some embodiments, the inhibitor of Ccdc141 signaling is a Ccdc141 inhibitor. In some embodiments, inhibition of Ccdc141 in CNS endothelial cells results in increased blood-CNS barrier permeability.

In some embodiments, the Ccdc141 inhibitor is capable of inhibiting Ccdc141 expression and/or activity. In some embodiments, the Ccdc141 inhibitor is an inhibitory nucleic acid targeting Ccdc141 mRNA. As described herein, an inhibitory nucleic acid, refers to nucleic acids capable of inhibiting expression or activity of a target gene (e.g., DNA, RNA, or protein of the target gene), for example, CCDCl41. Non-limiting examples of inhibitory nucleic acids include e.g., dsRNA, siRNA, shRNA, miRNA, amiRNA, antisense oligonucleotides (ASOs), DNA or RNA aptamers, et cetera An ASO is a small chain of nucleotides, generally 18-30 nucleotides long, that targets messenger RNA (mRNA) and is capable of altering mRNA expression through a variety of mechanisms, including ribonuclease H mediated decay of the pre-mRNA, direct steric blockage, and exon content modulation through splicing site binding on pre-mRNA. A small interfering RNA (siRNA), also known as short interfering RNA or silencing RNA, is a double-stranded non-coding RNA molecules, typically 20-27 base pairs in length, which interferes with the expression of specific genes with complementary nucleotide sequences by degrading mRNA after transcription, preventing translation. A short hairpin RNA or small hairpin RNA (shRNA) is an artificial RNA molecule with a tight hairpin turn that can be used to silence target gene expression via RNA interference (RNAi). A microRNA (abbreviated miRNA) is a small single-stranded non-coding RNA molecule (containing about 22 nucleotides) that functions in RNA silencing and post-transcriptional regulation of gene expression via base-pairing with complementary sequences within mRNA molecules. An amiRNA is an artificial miRNA. A mixmer is an oligomer consisting of alternating short stretch of LNA and DNA. An LNA, or Locked Nucleic Acid, also known as bridged nucleic acid (BNA), or inaccessible RNA, is a modified RNA nucleotide in which the ribose moiety is modified with an extra bridge connecting the 2′ oxygen and 4′ carbon. Aptamers are short sequences of artificial DNA or RNA that bind a specific target molecule.

In some embodiments, an inhibitory nucleic acid targeting Ccdc141 is an siRNA. siRNA molecules comprise a specific antisense sequence in addition to the reverse complement (sense) sequence.

The specificity of siRNA molecules may be determined by the binding of the antisense strand of the molecule to its target RNA (e.g., Ccdc141 mRNA). In some embodiments, the siRNA molecules are 60, 65, 70, 75, 80, 85, 90, 95, 100, or more base pairs in length. In some embodiments, the antisense sequence of the siRNA molecules is 7, 8, 9, 10, 11, 12, 13, 14, 15, 16, 17, 18, 19, 20, 21, 22, 23, 24, 25, 26, 27, 28, 29, 30, 35, 40, 45, 50, or more base pairs in length. In some embodiments, the antisense sequence of the siRNA molecules are 8 to 30 base pairs in length, 10 to 15 base pairs in length, 10 to 20 base pairs in length, 15 to 25 base pairs in length, 19 to 21 base pairs in length, or 21 to 23 base pairs in length.

Following selection of an appropriate target RNA sequence, siRNA molecules that comprise a nucleotide sequence complementary to all or a portion of the target sequence, i.e., an antisense sequence, can be designed and prepared using methods known in the art.

In some embodiments, the antisense sequence of the siRNA molecule is 7, 8, 9, 10, 11, 12, 13, 14, 15, 16, 17, 18, 19, 20, 21, 22, 23, 24, 25, 26, 27, 28, 29, 30, 35, 40, 45, 50, or more nucleotides in length. In some embodiments, the antisense sequence is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 19 to 21 nucleotides in length, or 21 to 23 nucleotides in lengths.

In some embodiments, the sense sequence of the siRNA molecule is 7, 8, 9, 10, 11, 12, 13, 14, 15, 16, 17, 18, 19, 20, 21, 22, 23, 24, 25, 26, 27, 28, 29, 30, 35, 40, 45, 50, or more nucleotides in length. In some embodiments, the sense sequence is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 19 to 21 nucleotides in length, or 21 to 23 nucleotides in lengths.

In some embodiments, siRNA molecules comprise an antisense sequence comprising a region of complementarity to a target region in a Ccdc141 mRNA (e.g., human Ccdc141 mRNA (SEQ ID NO: 1) or mouse Ccdc141 mRNA (SEQ ID NO: 2)). In some embodiments, the region of complementarity is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to a target region in a Ccdc141 mRNA (e.g., human Ccdc141 mRNA or mouse Ccdc141 mRNA). In some embodiments, the target region is a region of consecutive nucleotides in a CCDCl41 mRNA (e.g., human CCDCl41 mRNA or mouse Ccdc141 mRNA). In some embodiments, a complementary nucleotide sequence need not be 100% complementary to that of its target to be specifically hybridizable or specific for a Ccdc141 mRNA (e.g., human Ccdc141 mRNA or mouse Ccdc141 mRNA). Exemplary human CCDCl41 mRNA and mouse Ccdc141 mRNA sequences are set forth in SEQ ID NOs: 1 and 2.

In some embodiments, siRNA molecules comprise an antisense sequence that comprises a region of complementarity to in a Ccdc141 mRNA (e.g., human Ccdc141 mRNA or mouse Ccdc141 mRNA) sequence and the region of complementarity is in the range of 8 to 15, 8 to 30, 8 to 40, or 10 to 50, or 5 to 50, or 5 to 40 nucleotides in length. In some embodiments, the region of complementarity is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary to at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a Ccdc141 mRNA (e.g., human Ccdc141 mRNA (SEQ ID NO: 1) or mouse Ccdc141 mRNA (SEQ ID NO: 2)). In some embodiments, the region of complementarity comprises a nucleotide sequence that contains no more than 1, 2, 3, 4, or 5 base mismatches compared to the complementary portion of a Ccdc141 mRNA (e.g., human Ccdc141 mRNA or mouse Ccdc141 mRNA). In some embodiments, the region of complementarity comprises a nucleotide sequence that has up to 3 mismatches over 15 bases, up to 2 mismatches over 10 bases, or up to 1 mismatch over 5 bases.

Exemplary Ccdc141 target sequences are set forth below:

(SEQ ID NO: 9)
GGTAAGAATGGCATTAAATGGATCA
(SEQ ID NO: 10)
GAATGGCATTAAATGGATCACACCT
(SEQ ID NO: 11)
TGGCATTAAATGGATCACACCTTCT
(SEQ ID NO: 12)
GGCATTAAATGGATCACACCTTCTG
(SEQ ID NO: 13)
CATTAAATGGATCACACCTTCTGCA
(SEQ ID NO: 14)
TCACACCTTCTGCAGGGATACTCAT
(SEQ ID NO: 15)
ACACCTTCTGCAGGGATACTCATCA
(SEQ ID NO: 16)
CACCTTCTGCAGGGATACTCATCAA
(SEQ ID NO: 17)
CCTTCTGCAGGGATACTCATCAAAG
(SEQ ID NO: 18)
TCTGCAGGGATACTCATCAAAGTCA
(SEQ ID NO: 19)
GGTAAGAATGGCATTAAATGG
(SEQ ID NO: 20)
GGCATTAAATGGATCACACCT
(SEQ ID NO: 21)
GCAGGGATACTCATCAAAGTC

In some embodiments, siRNA molecules targeting Ccdc141 comprise an antisense strand which comprises a region of complementarity that is at least 60%, at least 65%, at least 70%, at least 75%, at least 80%, at least 85%, at least 90%, at least 95%, or 100% complementary to the Ccdc141 target sequences as set forth in any one of SEQ ID NOs: 9-21. In some embodiments, siRNA molecules targeting Ccdc141 comprise an antisense strand which comprises a region of complementarity that is complementary to at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides sequences as set forth in any one of SEQ ID NOs: 9-21.

In some embodiments, siRNA molecules targeting Ccdc141 comprise a sense strand which comprises a nucleotide sequence that is at least 60%, at least 65%, at least 70%, at least 75%, at least 80%, at least 85%, at least 90%, at least 95%, or 100% identical to the sequences as set forth in SEQ ID NOs: 9-18. In some embodiments, siRNA molecules targeting Ccdc141 comprise a sense strand at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of the sequence as set forth in SEQ ID NOs: 9-21.

In some embodiments, an inhibitory nucleic acid targeting Ccdc141 is an shRNA. shRNA molecules comprise a specific antisense sequence in addition to the reverse complement (sense) sequence, typically separated by a spacer or loop sequence. Cleavage of the spacer or loop provides a single-stranded RNA molecule and its reverse complement, such that they may anneal to form a dsRNA molecule (optionally with additional processing steps that may result in the addition or removal of one, two, three, or more nucleotides from the 3′ end and/or (e.g., and) the 5′ end of either or both strands). A spacer can be of a sufficient length to permit the antisense and sense sequences to anneal and form a double-stranded structure (or stem) prior to cleavage of the spacer (and, optionally, subsequent processing steps that may result in the addition or removal of one, two, three, four, or more nucleotides from the 3′ end and/or (e.g., and) the 5′ end of either or both strands). A spacer sequence may be an unrelated nucleotide sequence that is situated between two complementary nucleotide sequence regions which, when annealed into a double-stranded nucleic acid, comprise a shRNA.

The specificity of shRNA molecules may be determined by the binding of the antisense strand of the molecule to its target RNA sequence (e.g., Ccdc141 mRNA; SEQ ID NO: 1 or SEQ ID NO: 2). In some embodiments, the shRNA molecules are 60, 65, 70, 75, 80, 85, 90, 95, 100 or more base pairs in length. In some embodiments, the antisense sequence of the shRNA molecules is 7, 8, 9, 10, 11, 12, 13, 14, 15, 16, 17, 18, 19, 20, 21, 22, 23, 24, 25, 26, 27, 28, 29, 30, 35, 40, 45, 50, or more base pairs in length. In some embodiments, the antisense sequence of the shRNA molecules are 8 to 30 base pairs in length, 10 to 15 base pairs in length, 10 to 20 base pairs in length, 15 to 25 base pairs in length, 19 to 21 base pairs in length, or 21 to 23 base pairs in length.

Following selection of an appropriate target RNA sequence, shRNA molecules that comprise a nucleotide sequence complementary to all or a portion of the target sequence, i.e., an antisense sequence, can be designed and prepared using methods known in the art (see, e.g., Moore et al., Short Hairpin RNA (shRNA): Design, Delivery, and Assessment of Gene Knockdown, Methods Mol Biol. 2010; 629:141-158).

The shRNA molecules can comprise a hairpin (i.e., when two regions of the same strand, usually complementary in nucleotide sequence when read in opposite directions, base-pair to form a double helix that ends in an unpaired loop), or asymmetric hairpin (i.e., hairpin with a strand overhang) secondary structure, having self-complementary sense and antisense strands. In some embodiments, the shRNA targeting inhibitory nucleic acid described herein comprises an antisense sequence and a sense sequence.

In some embodiments, the antisense sequence of the shRNA molecule is 7, 8, 9, 10, 11, 12, 13, 14, 15, 16, 17, 18, 19, 20, 21, 22, 23, 24, 25, 26, 27, 28, 29, 30, 35, 40, 45, 50, or more nucleotides in length. In some embodiments, the antisense sequence is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 19 to 21 nucleotides in length, or 21 to 23 nucleotides in lengths.

In some embodiments, the sense sequence of the shRNA molecule is 7, 8, 9, 10, 11, 12, 13, 14, 15, 16, 17, 18, 19, 20, 21, 22, 23, 24, 25, 26, 27, 28, 29, 30, 35, 40, 45, 50, or more nucleotides in length. In some embodiments, the sense sequence is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 19 to 21 nucleotides in length, or 21 to 23 nucleotides in lengths.

In some embodiments, shRNA molecules comprise an antisense sequence comprising a region of complementarity to a target region in a Ccdc141 mRNA (e.g., human Ccdc141 mRNA or mouse Ccdc141 mRNA). In some embodiments, the region of complementarity is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the target region in a Ccdc141 mRNA (e.g., human Ccdc141 mRNA or mouse Ccdc141 mRNA) of SEQ ID NOs: 9-21. In some embodiments, the target region is a region of consecutive nucleotides in a Ccdc141 mRNA (e.g., human Ccdc141 mRNA or mouse Ccdc141 mRNA). In some embodiments, a complementary nucleotide sequence need not be 100% complementary to that of its target to be specifically hybridizable or specific for a Ccdc141 mRNA (e.g., human Ccdc141 mRNA or mouse Ccdc141 mRNA).

In some embodiments, shRNA molecules comprise an antisense sequence that comprises a region of complementarity in a Ccdc141 mRNA (e.g., human Ccdc141 mRNA or mouse Ccdc141 mRNA) sequence and the region of complementarity is in the range of 8 to 15, 8 to 30, 8 to 40, or 10 to 50, or 5 to 50, or 5 to 40 nucleotides in length. In some embodiments, the region of complementarity is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary to at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a Ccdc141 mRNA (e.g., human Ccdc141 mRNA or mouse Ccdc141 mRNA). In some embodiments, the region of complementarity comprises a nucleotide sequence that contains no more than 1, 2, 3, 4, or 5 base mismatches compared to the complementary portion of a Ccdc141 mRNA (e.g., human Ccdc141 mRNA or mouse Ccdc141 mRNA). In some embodiments, the region of complementarity comprises a nucleotide sequence that has up to 3 mismatches over 15 bases, up to 2 mismatches over 10 bases, or up to 1 mismatch over 5 bases.

In some embodiments, shRNA molecules targeting Ccdc141 comprises an antisense strand comprising a region of complementarity that is at least 60%, at least 65%, at least 70%, at least 75%, at least 80%, at least 85%, at least 90%, at least 95%, or 100% complement to the sequences as set forth in any one of SEQ ID NOs: 9-21. In some embodiments, shRNA molecules targeting Ccdc141 comprise an antisense strand comprising a region of complementarity that is complementary to at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of the sequences as set forth in any one of SEQ ID NOs: 9-21.

In some embodiments, the inhibitory nucleic acids targeting Ccdc141 can be administered to the subject using any suitable known method, for example, but not limited to, direct injection, viral vector mediated delivery (e.g., AAV, retrovirus, adeno virus, or lentivirus), or ceDNA. In some embodiments, the Ccdc141 inhibitor is an antibody, an antibody variant or an antigen-binding fragment thereof targeting Ccdc141. An antibody: As used herein, the term “antibody” refers to a polypeptide that includes at least one immunoglobulin variable domain or at least one antigenic determinant, e.g., paratope that specifically binds to an antigen. In some embodiments, an antibody is a full-length antibody. In some embodiments, an antibody is a chimeric antibody. In some embodiments, an antibody is a humanized antibody. However, in some embodiments, an antibody is a Fab fragment, a F(ab′) 2 fragment, a Fv fragment or a scFv fragment. In some embodiments, an antibody is a nanobody derived from a camelid antibody or a nanobody derived from shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (a), delta (A), epsilon (€), gamma (γ) or mu (u) heavy chain. In some embodiments, the heavy chain of an antibody described herein can comprise a human alpha (a), delta (4), epsilon (E), gamma (Y) or mu (u) heavy chain. In a particular embodiment, an antibody described herein comprises a human gamma 1 CH1, CH2, and/or CH3 domain. In some embodiments, the amino acid sequence of the VH domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) NIH publication no. 91-3242. In some embodiments, the VH domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P., et a1. (1993) Proc. Natl. Acad. Sci. USA 90:6444-6448; Poljak, R. J., et a1. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et a1. (1995) Human Antibodies and Hybridomas 6:93-101) and use of a cysteine residue, a marker peptide and a C-terminal polyhistidine tag to make bivalent and biotinylated scFv molecules, Mol. Immunol. 31:1047-1058). Antibodies, antibody variants and antigen-binding fragments targeting Ccdc141 have been previously described, see, e.g., PA5-21169, PA5-116237 from Invitrogen, DPABH-17228, DPABH-24009, DPAB-L20267 from Creative Diagnostics, 6399 from ProSci Inc. Non-limiting examples of anti-Ccdc 141 antibody include LS-B9522 from LS Bio; CAT #: AP50767PU-N, CAT #: TA320162, CAT #: TA334850 from Origene.

In some embodiments, administration of an effective amount of any one of the Ccdc141 inhibitors described herein or a combination thereof results in increased blood-CNS barrier permeability.

In some embodiments, an agent that promotes Pacsin2 expression and/or activity at the blood-CNS barrier is an isolated nucleic acid encoding Pacsin2. In some embodiments, promoting expression/activity in CNS endothelial cells results in increased blood-CNS barrier permeability.

In some embodiments, an agent that promotes the Pacsin2 signaling at the blood-CNS barrier is a recombinant human Pacsin2 or a fragment thereof. In some embodiments, the present disclosure provides a recombinant human Pacsin2 comprises an amino acid sequence. An exemplary human Pacsin2 amino acid sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% identical to the amino acid sequence as set forth in SEQ ID NO: 7 (NP_001171899.1). In some embodiments, an exemplary human Pacsin2 amino acid sequence is set forth in SEQ ID NO: 7 (NP_001171899.1):

MSVTYDDSVGVEVSSDSFWEVGNYKRTVKRIDDGHRLCSDLMNCLHERAR
IEKAYAQQLTEWARRWRQLVEKGPQYGTVEKAWMAFMSEAERVSELHLEV
KASLMNDDFEKIKNWQKEAFHKQMMGGFKETKEAEDGFRKAQKPWAKKLK
EVEAAKKAHHAACKEEKLAISREANSKADPSLNPEQLKKLQDKIEKCKQD
VLKTKEKYEKSLKELDQGTPQYMENMEQVFEQCQQFEEKRLRFFREVLLE
VQKHLDLSNVAGYKAIYHDLEQSIRAADAVEDLRWFRANHGPGMAMNWPQ
FEEWSADLNRTLSRREKKKATDGVTLIGINQTGDQSLPSKPSSTLNVPSN
PAQSAQSQSSYNPFEDEDDTGSTVSEKDDTKAKNVSSYEKTQSYPTDWSD
DESNNPFSSTDANGDSNPFDDDATSGTEVRVRALYDYEGQEHDELSFKAG
DELTKMEDEDEQGWCKGRLDNGQVGLYPANYVEAIQ

In some embodiments, an agent that promotes the Pacsin2 signaling at the blood-CNS barrier is a recombinant mouse Pacsin2 or a fragment thereof. In some embodiments, the recombinant mouse Pacsin2 comprises an amino acid sequence. An exemplary mouse Pacsin2 amino acid sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% identical to the amino acid sequence as set forth in SEQ ID NO: 8 (NP_001152981.1). In some embodiments, an exemplary mouse Pacsin2 amino acid sequence is set forth in SEQ ID NO: 8

### (NP_001152981.1):

MSVTYDDSVGVEVSSDSFWEVGNYKRTVKRIDDGHRLCGDLMNCLHERAR
IEKAYAQQLTEWARRWRQLVEKGPQYGTVEKAWIAVMSEAERVSELHLEV
KASLMNEDFEKIKNWQKEAFHKQMMGGFKETKEAEDGFRKAQKPWAKKLK
EVEAAKKAHHTACKEEKLAISREANSKADPSLNPEQLKKLQDKIEKCKQD
VLKTKDKYEKSLKELDQTTPQYMENMEQVFEQCQQFEEKRLRFFREVLLE
VQKHLDLSNVASYKTIYRELEQSIKAADAVEDLRWFRANHGPGMAMNWPQ
FEEWSADLNRTLSRREKKKAVDGVTLTGINQTGDQSGQNKPGSNLSVPSN
PAQSTQLQSSYNPFEDEDDTGSSISEKEDIKAKNVSSYEKTQTYPTDWSD
DESNNPFSSTDANGDSNPFDEDTTSGTEVRVRALYDYEGQEHDELSFKAG
DELTKIEDEDEQGWCKGRLDSGQVGLYPANYVEAIQ

In some embodiments, an agent that promotes the Pacsin2 signaling at the blood-CNS barrier is a nucleic acid encoding Pacsin2 (e.g., human or mouse Pacsin2). In some embodiments, the nucleic acid encoding Pacsin2 encodes a mammalian Pacsin2 (e.g., human Pacsin2, mouse Pacsin2, rat Pacsin2, non-human primate Pacsin2, etc.). In some embodiments, the nucleic acid encoding Pacsin2 encodes a human Pacsin2. In some embodiments, the nucleic acid encoding Pacsin2 encodes a mouse Pacsin2.

Human Pacsin2 mRNA sequence is set forth in SEQ ID NO: 5 (NM_001184970.3). In some embodiments, the nucleic acid encoding human Pacsin2 comprises a nucleic acid sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% identical to the nucleotide sequence of SEQ ID NO: 5. The nucleotide sequence encoding human Pacsin2 includes NM_001184970.3. An exemplary nucleotide sequence encoding human Pacsin2 protein is set forth in SEQ ID NO: 5 (NM_001184970.3):

AGAAGCGGCCGCAGGGGTCTGGGCAGGGCTGGGCAGTGCTGCCGGAGCAAAAGCGGTAGCGG
GAGCCCGGCCGGAGCTGGGTCTGGAGACGCCGTGGCAGCCTGAACGGAGTGTGCGACGGATT
GGGAGGTTTGTCTACAGATTTTGAGCGTTCGAAGTTGACCCCTGACTAAGTATACTTTGCTG
CTCCCTCAGCCTTTGAAAAAATGTCTGTCACATATGATGATTCCGTTGGAGTAGAAGTGTCC
AGCGACAGCTTCTGGGAGGTCGGGAACTACAAGCGGACTGTGAAGCGGATCGACGATGGCCA
CCGCCTGTGCAGCGACCTCATGAACTGCCTGCATGAGCGGGCGCGCATCGAGAAGGCGTATG
CGCAGCAGCTCACTGAGTGGGCCCGGCGCTGGAGGCAGCTCGTGGAGAAAGGGCCCCAGTAC
GGGACCGTGGAGAAGGCCTGGATGGCCTTCATGTCCGAGGCAGAGAGGGTGAGCGAGCTGCA
CCTCGAGGTGAAGGCCTCACTGATGAACGATGACTTCGAGAAGATCAAGAACTGGCAGAAGG
AAGCCTTTCACAAGCAGATGATGGGCGGCTTCAAGGAGACCAAGGAAGCTGAGGACGGCTTT
CGGAAGGCACAGAAGCCCTGGGCCAAGAAGCTGAAAGAGGTAGAAGCAGCAAAGAAAGCCCA
CCATGCAGCGTGCAAAGAGGAGAAGCTGGCTATCTCACGAGAAGCCAACAGCAAGGCAGACC
CATCCCTCAACCCTGAACAGCTCAAGAAATTGCAAGACAAAATAGAAAAGTGCAAGCAAGAT
GTTCTTAAGACCAAAGAGAAGTATGAGAAGTCCCTGAAGGAACTCGACCAGGGCACACCCCA
GTACATGGAGAACATGGAGCAGGTGTTTGAGCAGTGCCAGCAGTTCGAGGAGAAACGCCTTC
GCTTCTTCCGGGAGGTTCTGCTGGAGGTTCAGAAGCACCTAGACCTGTCCAATGTGGCTGGC
TACAAAGCCATTTACCATGACCTGGAGCAGAGCATCAGAGCAGCTGATGCAGTGGAGGACCT
GAGGTGGTTCCGAGCCAATCACGGGCCGGGCATGGCCATGAACTGGCCGCAGTTTGAGGAGT
GGTCCGCAGACCTGAATCGAACCCTCAGCCGGAGAGAGAAGAAGAAGGCCACTGACGGCGTC
ACCCTGACGGGCATCAACCAGACAGGCGACCAGTCTCTGCCGAGTAAGCCCAGCAGCACCCT
TAATGTCCCGAGCAACCCCGCCCAGTCTGCGCAGTCACAGTCCAGCTACAACCCCTTCGAGG
ATGAGGACGACACGGGCAGCACCGTCAGTGAGAAGGACGACACTAAGGCCAAAAATGTGAGC
AGCTACGAGAAGACCCAGAGCTATCCCACCGACTGGTCAGACGATGAGTCTAACAACCCCTT
CTCCTCCACGGATGCCAATGGGGACTCGAATCCATTCGACGACGACGCCACCTCGGGGACGG
AAGTGCGAGTCCGGGCCCTGTATGACTATGAGGGGCAGGAGCATGATGAGCTGAGCTTCAAG
GCTGGGGATGAGCTGACCAAGATGGAGGACGAGGATGAGCAGGGCTGGTGCAAGGGACGCTT
GGACAACGGGCAAGTTGGCCTATACCCGGCAAATTATGTGGAGGCGATCCAGTGATGAGTCG
GGGACAGGCCAGCGGGGGGACGGAGGCGGCGGGCCCAGGAGCCTCAGCCAGCCACGTGGGCA
TCCACTCCTTTTCCTGCAAGAGATGATGGTTCCATTGCTCTTGGCTTCATGGTGTTCCTGGA
AGGCAGATGAGCTGGTCATTTCGCCTGGGACTCGGCACCTTTCCGAGTGCAGCTGGAGGGAT
CTGAGCGCAGGAAGACGCAGAACAACAGAAATAGCCGCCCCTCCCCGCCCACTGTGCCTGTT
GGCCTATCATAGATCTCTATGTTCTTGACTTTGTCTCTCCTTTCCGAGTCAATGGTGGGTTA
CACTGATCTTGTTCCACTGATTACTCTCTCTGACGAGTCCATCACCTGCAACTTAAATGAAC
AAGCTTACATCCCATTTTGAGTGAAGATTTTGAGGTTTTTAATTTAAAGGCTGTGTACAGTT
ATACTTTTTTATACACCTGTTCATTTCTACTTAAATTATGGCACAGATTGATGCGCACCAGT
CTTGAGGAAACGATCTCCCTATTCCCTTACCCTGTTACTCAGCCACGCCGTGTGTAGGCTTA
GCCTCAGGTGGCAGATGTTTGAGGAAAGGAATTATGCCAGGAAGGTGGGACCGGGTTATGGT
CGGGTTTCTATTGGGAATGCTCTTTGTGCTTTTGGGCATCTGAATGGAAGCTTTACATAGAA
CCTTAGGTAGAACTCCCCCAAATCGCCATATTTAAAAATTATTTTCACTCTATTCTTGCTTA
AAACTGTACTCTTTTGCAAATTAACAATTTTATCACTGATTCAGAGTTAAAAAGAAGACTAA
CTTTTCAAGCAAATGCATCTGTAAAGATGCTTTAGATTAGACTGTCATGTCTCAGTGTCTAT
CTGTATATATTATTTGATATTCAGAGAATCTAAAGCACTCGTCTACTGTTTTAATGAGATTT
AACAGCTTTTAACAGTGAGTTTCGTTTGTAAACTGCTTGAAGTCTGTGGCATTCAGGCACAC
GTCTGGCTGGCCGGCTGGGTCTCCTCCCGGGCTCAGTGGGCCTGGGGCCTCTCTGACGTGGT
GCCTGCTGGAGGGAGGCTCGTCGTCACCAGCTGACTGCTGGTCCGGCTTCTGACCGGCCTTT
GTCCTGGCTCCGTAGCAGAACACTGTAAAAGTGCCCGCGTCTTTGCAGTAGTTGCAGATTTC
AGTCGTCGTGTTACTTGTGCACAAACAGAAGCTGGGTCTTACCCGCAGCACGAGTGTCTCGG
GCTGCCCGGAGTCGCCCGGGAGCAGGTGCTGCAGCCAGAGTTACGCGGGGGCCACGCGGGCC
GGCGGGGGTGGGGGGAACGTGGGGGAACCTGTGTTTCACGTGACTCAGCAGTGCCCGCCGCC
GTCACCAGCTATGCATTCACTCCGTTTCCAGTGAGCAGATGTCTTGCTTGGAAAGTGGACCT
GTGTCTGTGTCTGTCCTGAGAACTTACCAGCAGAAATCCTCATTTCTGTGCTACGGATTTAC
CAAAAATTGTCAAGTCTTTTTCAGTTTAACAGTTCCTTTACATGTGTAGTATTTGAGGAAAA
AAATCAATAAACAGTTGATCTCGTGCA

Mouse Pacsin2 mRNA sequence is set forth in SEQ ID NO: 6 (NM_001159509.1). In some embodiments, the nucleic acid encoding mouse Pacsin2 comprises a nucleic acid sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% identical to the nucleotide sequence of SEQ ID NO: 6. An exemplary nucleotide sequence encoding mouse Pacsin2 protein is set forth in SEQ ID NO: 6 (NM_001159509.1):

GCGGCGCGGTCGCGGCTGGCGGCAGAAGCGGCTGCGGCGGGTCTGGGCAGGGCCGGGGAGCT
CGGTTGGAGCGGAACGGGAAGGGGTCGGAGTCGCAGCCCGGCCGGGGCGGCGTGCTCAGTGG
GACCCGAGGCGAACGGCGGCCGAGGTCTGTCTGCAGGTTTTACACATCTGACCCCTGAACTG
GAGTGTCTGCTGCCACCCCCTCGTCCTCTGCAGAAATGTCTGTCACCTACGATGACTCTGTG
GGAGTGGAAGTGTCCAGCGACAGCTTCTGGGAGGTTGGGAACTACAAACGGACTGTGAAGCG
GATTGACGATGGCCACCGCCTGTGTGGTGACCTCATGAACTGTCTGCATGAGCGGGCACGCA
TCGAGAAGGCGTATGCACAGCAGCTCACTGAGTGGGCCCGACGCTGGAGGCAGCTGGTAGAG
AAGGGACCACAGTATGGGACCGTGGAGAAGGCCTGGATAGCTGTCATGTCTGAAGCAGAGAG
GGTGAGTGAACTGCACCTGGAAGTGAAGGCATCACTGATGAATGAAGACTTTGAGAAGATCA
AGAACTGGCAGAAGGAAGCCTTTCACAAGCAGATGATGGGAGGCTTCAAGGAGACCAAAGAA
GCAGAGGATGGCTTTCGGAAGGCCCAGAAGCCCTGGGCCAAGAAGCTGAAAGAGGTGGAAGC
GGCAAAGAAGGCGCACCACACAGCGTGCAAAGAGGAGAAGCTGGCCATCTCCCGGGAAGCCA
ACAGCAAGGCAGATCCATCCCTCAACCCTGAGCAGCTGAAGAAACTGCAAGACAAGATAGAA
AAATGCAAACAGGACGTTCTAAAGACCAAGGACAAGTATGAGAAGTCCCTGAAGGAGCTTGA
TCAGACCACACCCCAGTACATGGAGAACATGGAGCAGGTGTTCGAGCAGTGCCAGCAGTTTG
AAGAGAAGCGCCTGCGCTTCTTCCGGGAGGTTCTGCTGGAGGTTCAGAAGCACTTGGATCTG
TCCAATGTGGCTAGCTATAAAACCATTTACCGGGAGCTGGAGCAGAGCATCAAAGCAGCAGA
TGCGGTAGAGGACCTGAGGTGGTTCCGGGCTAACCATGGGCCAGGCATGGCTATGAACTGGC
CACAGTTTGAGGAGTGGTCTGCAGATCTGAATCGAACTCTCAGCCGGAGAGAGAAGAAGAAG
GCTGTTGACGGTGTCACCCTAACAGGGATCAACCAGACAGGTGACCAGTCTGGACAGAACAA
GCCTGGCAGCAACCTTAGTGTCCCGAGCAACCCCGCCCAGTCCACGCAGTTACAGTCCAGCT
ACAACCCCTTCGAGGACGAGGACGACACGGGCAGCAGCATCAGTGAGAAGGAGGACATTAAG
GCCAAAAATGTCAGCAGCTATGAGAAGACTCAGACTTACCCCACTGACTGGTCTGATGATGA
GTCTAACAACCCTTTCTCCTCCACGGATGCCAACGGGGATTCGAACCCATTTGATGAGGACA
CGACCTCAGGAACAGAAGTGCGAGTTCGGGCCCTCTATGACTATGAGGGGCAGGAACATGAT
GAGCTGAGCTTCAAGGCTGGGGATGAACTGACCAAGATAGAGGATGAAGATGAACAGGGTTG
GTGCAAGGGACGTTTAGACAGCGGCCAGGTTGGCCTATACCCAGCCAACTATGTCGAGGCTA
TCCAGTGACAGCCCATGGGCAGGCTGGCGGAGAGACGGAAATGGGCAGTTCAGGAGCTCCGT
TAGCCTTGGCCTGGGCAGTGACACCTCTAGTGCCCCCAGCAGCCATGTAGGCATCCACTCCA
CCTGCAAAAGACGATGGCTCTGTTGTTCTTGGCTTCCTGGTGTGCTTTGAAGGCAGATGAGC
TGGTGATTTCATTGGGCACTTGGCCCTTTTCCAAGCACATCTGGGCAGATATAGACACAGGA
AGATAGGGTCCAACAGCGAGAGCCAGGCCCCTCCCCACCCCCACCAGCTCTCTCTATCATGG
ATCTGCACCTTCTCGCCCTTGTCTCTCCTGAGTCATGACGGGTCATACTGATTCTTGTTCCA
CTGATGATTTTCTCTGATGAGGTCCTATCTGCAAGGTCAATGAGCAGACTTACATGCCATCT
TCTGAGTAAAGAGTTTGAGGTTTTAATTTAAAGGCAATGTACAGCTATACTTTTTTATATGC
TCTTCCAGTCAGTTAAATTATGGCCTACACTGATCTGAGATGTTCTCCACGTGAGCTGTCTT
CATTTCTCTGTGCTATGTCCAGATGTGGGGTTGCTGCAGCCGGGGTTCTATGGCAAGTGCCA
GTTGCAGGGCTAACCTTGTGCAACGTTCCCCAACACTTCCACATACAGAAATTATTTTCACT
CTATCCCTGCTTCAGTTTTTGCAGATTAACAGTTCTATTAGTGATTTGGAAAGTTAACAGTA
AGAAGACTAACTTTTCAAAACAGTTGCATCTGTAGATTAAGATGCTTTACATTAGACCGTTG
TGTCTCGATGTATATCTGTATATATTATTTGATAATCAGAAAATCTATAGAGTTCACCCACT
GTTGAATGAGAGCTGGTGGCTTCTGACAGCAGATCTGGTCAACTGCTTGAAGCCCATGGCAT
TGAAGCACAGGCACGGCTGGTTAACGGTGCCCACCCAGTTAGGATGTGGCTCTGGCCTCTGA
GTGGAGCTGCTGGGAAGACTGATTCTCATTGGCCTGGGCTCCAAGCTCATGACCGAGCACTG
GAAAAGCTCCTAGGACTTGGTAGTAATCGTAGACTTCACAGTCCCTGTGTCACTCACTGGAG
AGCTAGAGGGAGGGGTTCGACACCCTCCACCACACACACACACACACACACACACACACACA
CAAGTTCCTCCAGTTGCCCTTGTCCTCAGGTGCAGTGGGACTGTTGTGAGCCCCAGGGATGG
GCACAAAGAGGACTTTTATTTTGTTAGCTCGGACAGTGCAGTGGTGCACATCAGCAACTTGT
ATTTCTTCGGTGTTTGGCACGAGCACTGTCTCGCTGTGGCTGTGTGTCATGAGAACTTACCA
GCAGAAATCCTTGTTCCTAAGCTACAGAATGACCAAAAGCTGTCAAGTCCTTAATGTTTAGA
AACTCCTTAAAATGTATAGTATTTTAGAACAACAACAACAAAACTCAATAAACAGTTGATCT
TGTGTGTTTGACAGTCCCTTTGCCTTGCATCCACCCTCATGTTCACACAGCTGATAGACCTG
TCTCCTTGGGGGTGTACCAGCTTTGAAACTGGGCAAGATCCAGTTCATAGGATCCCTTCTCG
GGACCATGTCTCAGTTCCTGCACCAGAGGCTGGGGACTTGGGGCACCTTGGGCATCCCTGAG
GAGGGCATCCCTGGTGCCACCCTCTCTCCCCTGACCAACAACCGCTCCTCCCCAAACCTGCG
TGTGTTGACCTTTATGTGCCACTGTTCTGTTTTCTCAAGCTGATGATCTCTGGGTGTAATGT
GATCAATGCAGGTCCCAGCTGGTCCTGCTCGCACAAAGAACCTCCAAGGATCTTGCCTCCTA
TTGGGATCAGCTAAGGAGATAAAGCCAGTGCGGTAGCCCTGACCAGTCCCGGTGAACGGACT
GCTGCTGCTGCTGCTGCTGCTGCTGCTGCTGCTGCTGCTGCTGCTGCTGCTGCTGCTGCTGC
TCTTCAACACTAGTAAAGCTTCTCTCTGGCCTTCC

An effective amount of an agent that increases blood-CNS barrier permeability (e.g., e.g., Ccdc141 inhibitor or an agent that promotes Pacsin2 expression/activity) may be an amount sufficient to have a therapeutic benefit in a subject, e.g., to extend the lifespan of a subject, to improve and/or reverse in the subject one or more symptoms of disease, or to slow disease progression. The effective amount will depend on a variety of factors such as, for example, the species, age, weight, health of the subject, and the tissue to be targeted, and may thus vary among subject and tissue. An effective amount may also depend on the inhibitor used.

In some embodiments, administration of the inhibitors described herein may result in inhibition of Ccdc141 signaling of one or more of the CNS endothelial cells. In some embodiments, administration of the inhibitors described herein may result in inhibition of Ccdc141 signaling in all of the CNS endothelial cells.

In some embodiments, administration of an agent promoting Pacsin2 expression/activity described herein may result in increasing of Pacsin2 signaling in one or more of the CNS endothelial cells. In some embodiments, administration of an agent promoting Pacsin2 expression/activity described herein may result in increasing of Pacsin2 signaling in all of the CNS endothelial cells.

An effective amount may also depend on the mode of administration. For example, targeting endothelial cells in the CNS by intravenous administration or subcutaneous injection may require different (e.g., higher, or lower) doses, in some cases, than targeting endothelial cells in the CNS by another method (e.g., local injection to the CNS). In some embodiments, the inhibitor (e.g., Ccdc141 inhibitor) described herein is a small molecule and can be taken orally. In some embodiments, an agent promoting Pacsin2 expression/activity is an rAAV encoding Pacsin2.

In some embodiments, administering an inhibitor of Ccdc141 signaling decreases the level and/or activity of Ccdc141. In some embodiments, administration of an Ccdc141inhibitor described herein decreases the expression and/or activity of Ccdc141 by at least 10% or more, e.g., by 10% or more, 50% or more, 100% or more, 200% or more, 500% or more, or 1000% or more. In some embodiments, administration of an inhibitors described herein results in increasing of the permeability of the blood-CNS barrier in the subject. In some embodiments, administration of an inhibitor described herein results in increasing the permeability of the blood-CNS barrier in the subject by at least 10%, at least 15%, at least 20%, at least 25%, at least 30%, at least 35%, at least 40%, at least 45%, at least 50%, at least 55%, at least 60%, at least 65%, at least 70%, at least 80%, at least 85%, at least 90%, at least 95%, at least 99%, at least 100%, at least 150%, at least 200%, at least 300%, at least 400%, at least 500% or more compared the permeability of the blood-CNS barrier of the subject prior to administration of the inhibitor.

In some embodiments, administering an agent that promotes Pacsin2 expression/activity (e.g., rAAV encoding Pacsin2). In some embodiments, administration of an agent that promotes Pacsin2 expression/activity (e.g., rAAV encoding Pacsin2) by at least 10% or more, e.g., by 10% or more, 50% or more, 100% or more, 200% or more, 500% or more, or 1000% or more. In some embodiments, administration of an agent that promotes Pacsin2 expression/activity (e.g., rAAV encoding Pacsin2) described herein results in increasing of the permeability of the blood-CNS barrier in the subject. In some embodiments, administration of an agent that promotes Pacsin2 expression/activity (e.g., rAAV encoding Pacsin2) described herein results in increasing the permeability of the blood-CNS barrier in the subject by at least 10%, at least 15%, at least 20%, at least 25%, at least 30%, at least 35%, at least 40%, at least 45%, at least 50%, at least 55%, at least 60%, at least 65%, at least 70%, at least 80%, at least 85%, at least 90%, at least 95%, at least 99%, at least 100%, at least 150%, at least 200%, at least 300%, at least 400%, at least 500%, or more compared the permeability of the blood-CNS barrier of the subject prior to administration of the inhibitor.

The permeability of blood-CNS barrier can be measured using any suitable technique or method known in the art, e.g., neuroimaging techniques including dynamic perfusion CT (PCT) and dynamic contrast-enhanced magnetic resonance imaging (DCEMRI), quantification of protein biomarkers (e.g., neuron-specific enolase (NSE), glial fibrillary acidic protein (GFAP), and S100β) in the cerebral spinal fluid (CSF), et cetera.

In some embodiments, the methods described herein relate to treating a subject having or diagnosed as having a disease affecting the CNS, e.g., a neurological disease or a condition treated by delivering therapeutic agents suitable for treating the suspected or diagnosed disease to the CNS. Subjects having a disease affecting the CNS can be identified by a physician using current methods of diagnosing such conditions. Symptoms and/or complications of such conditions which characterize these conditions and aid in diagnosis are known in the art and include, but are not limited to, loss of neural function (e.g., lack of coordination, lack of sensation, altered behaviors, inflammation of the CNS, headaches, et cetera). Tests that may aid in a diagnosis of such conditions can include, but are not limited to, CT scan, MRI scan, spinal tap, brain biopsy, nerve biopsy, electroencephalogram (EEG), lumbar puncture, physical examination, nerve conduction studies, and/or blood tests. For some conditions, a family history of the condition (e.g., by genetic analysis), or exposure to risk factors for the condition can also aid in determining if a subject is likely to have the condition or in making a diagnosis.

In some embodiments, compositions for increasing blood-CNS permeability (e.g., Ccdc141 inhibitors or rAAV encoding Pacsin2) described herein can be administered to a subject having or diagnosed as having a disease affecting the CNS. In some embodiments, the methods described herein comprise administering an effective amount of a composition for increasing blood-CNS permeability (e.g., Ccdc141 inhibitors or rAAV encoding Pacsin2) described herein or a composition thereof, to a subject in order to alleviate a symptom of a disease affecting the CNS. As used herein, “alleviating a symptom” is ameliorating any condition or symptom associated with the disease affecting the CNS. As compared with an equivalent untreated control, such reduction is by at least 5%, 10%, 20%, 40%, 50%, 60%, 80%, 90%, 95%, 99% or more as measured by any standard technique.

In some embodiments, a composition for increasing blood-CNS permeability (e.g., an inhibitor of Ccdc141 signaling or rAAV encoding Pacsin2) can be administered to a subject in need thereof in combination with a therapeutic agent to the central nervous system. In some embodiments, the administration of a composition for increasing blood-CNS permeability (e.g., an inhibitor of Ccdc141 signaling or rAAV encoding Pacsin2) at the blood-CNS barrier increases blood-CNS barrier permeability thereby facilitating the delivery of the therapeutic agent to the CNS. The therapeutic agent can be any agent for the treatment of any disease, provided that it is desired that the therapeutic agent reaches the central nervous system. In some embodiments, methods which comprise administering an inhibitor of Ccdc141 signaling can further comprise administering a therapeutic agent to the subject. In some embodiments, the present disclosure provides a methods for delivering a therapeutic agent to the CNS, the method comprising administering to the subject (i) a therapeutic agent, and (ii) a composition for increasing blood-CNS permeability (e.g., an inhibitor of Ccdc141 signaling or rAAV encoding Pacsin2). In some embodiments, a therapeutic agent and a composition for increasing blood-CNS permeability (e.g., an inhibitor of Ccdc141 signaling or rAAV encoding Pacsin2) are administered sequentially (e.g., administration of a composition for increasing blood-CNS permeability prior to administration of the therapeutic agent). In some embodiments, a therapeutic agent and a composition for increasing blood-CNS permeability (e.g., an inhibitor of Ccdc141 signaling or rAAV encoding Pacsin2) are administered concurrently (e.g., administration of a composition for increasing blood-CNS permeability and the therapeutic agent at the same time in one formulation or separate formulations). In some embodiments, a therapeutic agent and a composition for increasing blood-CNS permeability (e.g., an inhibitor of Ccdc141 signaling or rAAV encoding Pacsin2) are administered at different frequencies (e.g., administration of a composition for increasing blood-CNS permeability once followed by administration of the therapeutic agent multiple times). Non-limiting examples of such therapeutic agents can include, antibiotics, antibodies, anticonvulsant (e.g., gabapentin), chemotherapeutics, anti-inflammatoireurotransmitters, pain medication (e.g., morphine), peptides, nucleic acids (e.g., RNAi-based therapies), or psychiatric drugs. In some embodiments, a subject in need of increased permeability of the blood-brain barrier is in need of treatment for a condition selected from the group consisting of neuromuscular diseases (e.g., Amyotrophic Lateral Sclerosis (ALS), Ataxia, Cerebral Palsy, Muscular Dystrophy), neurodegenerative disease (e.g., Alzheimer's Disease, Parkinson's disease, motor neuron disease, Amyotrophic lateral sclerosis, spinal muscular atrophy, spinocerebral ataxia.), brain and nerve tumors (e.g., Chordomas, Craniopharyngiomas, Gangliocytomas, Glomus jugulare, Meningiomas, Pineocytomas, Pituitary adenomas, Schwannomas, Gliomas, Astrocytomas, Ependymomas, Glioblastoma multiforme (GBM), Medulloblastomas, Oligodendrogliomas, Hemangioblastomas, Rhabdoid tumors, medulloblastomas, low-grade astrocytomas (pilocytic), ependymomas, craniopharyngiomas and brainstem gliomas, et cetera), Neurogenetic Diseases (e.g., Ataxia including spinocerebellar ataxias, olivopontocerebellar atrophies, and multiple system degeneration, CADASIL (Cerebral Autosomal Dominant Arteriopathy with Sub-cortical Infarcts and Leukoencephalopathy), Charcot-Marie-Tooth disease (hereditary neuropathy), Cognitive disorders (e.g., familial Alzheimer's disease and other familial dementias including frontotemporal dementia, familial Pick's disease, familial Creutzfeldt-Jakob disease, Familial amyotrophic lateral sclerosis (familial ALS also known as Lou Gehrig's disease), Familial dystonia including Dopa-responsive dystonia, Fragile X and Fragile X associated Tremor Ataxia Syndrome (FXTAS), Hereditary Spastic Paraplegia, Huntington's disease, Leukodystrophy including adrenomyeloneuropathy and cerebrotendinous xanthomatosis, Lysosomal storage disorders including Gaucher, Niemann-Pick, and Fabry diseases, Mitochondrial encephalomyopathies (MELAS syndrome), Mucopolysaccharidoses, Neurofibromatosis, Primary Lateral Sclerosis, Tourette's syndrome, Tuberous sclerosis, Von-Hippel-Lindau disease, Wilsons disease), or neuropsychiatric disorders (e.g., schizophrenia (SZ), bipolar disorder (BD), major depressive disorder (MDD) and attention deficit hyperactivity disorder (ADHD), et cetera). The identity of such CNS therapeutic agents is known in the art and described, e.g., in Ghose et al., J Comb Chem 1999 1:55-68 and Pardridge. NeuroRx 2005 2:3-14; each of which is incorporated by reference herein in its entirety. In some embodiments, a central nervous system therapeutic agent can inhibit the activity and/or expression of a therapeutic target gene associated with a central nervous system disease (e.g., examples of such genes are described below herein), e.g., it can be an inhibitory nucleic acid or an inhibitory antibody reagent.

In some embodiments, the central nervous system therapeutic agent is less than about 1000 kDa in size. In some embodiments, the central nervous system therapeutic agent is less than about 500 kDa in size. In some embodiments, the central nervous system therapeutic reagent is less than about 300 kDa in size. In some embodiments, the central nervous system therapeutic reagent is less than about 200 kDa in size. In some embodiments, the central nervous system therapeutic reagent is less than about 70 kDa in size. In some embodiments, the central nervous system therapeutic reagent is less than about 50 kDa in size. In some embodiments, the central nervous system therapeutic reagent is less than about 20 kDa in size. In some embodiments, the central nervous system therapeutic reagent is less than about 10 kDa in size. In some embodiments, the central nervous system therapeutic reagent is less than about 1 kDa in size. In some embodiments, a CNS therapeutic agent is between 1 and 1000 kDa in size, between 5 and 1000 kDa in size, between 10 and 1000 kDa in size, between 20 and 1000 kDa in size, between 50 and 1000 kDa in size, between 100 and 1000 kDa in size, between 150 and 1000 kDa in size, between 200 and 1000 kDa in size, between 300 and 1000 kDa in size, between 400 and 1000 kDa in size, between 500 and 1000 kDa in size, between 600 and 1000 kDa in size, between 700 and 1000 kDa in size, between 800 and 1000 kDa in size, between 900 and 1000 kDa in size, between 1 and 750 kDa in size, between 5 and 750 kDa in size, between 10 and 750 kDa in size, between 20 and 750 kDa in size, between 50 and 750 kDa in size, between 100 and 750 kDa in size, between 150 and 750 kDa in size, between 200 and 750 kDa in size, between 300 and 750 kDa in size, between 400 and 750 kDa in size, between 500 and 750 kDa in size, between 600 and 750 kDa in size, between 700 and 750 kDa in size, between 1 and 500 kDa in size, between 5 and 500 kDa in size, between 10 and 500 kDa in size, between 20 and 500 kDa in size, between 50 and 500 kDa in size, between 100 and 500 kDa in size, between 150 and 500 kDa in size, between 200 and 500 kDa in size, between 300 and 500 kDa in size, between 400 and 500 kDa in size, between 1 and 250 kDa in size, between 5 and 250 kDa in size, between 10 and 250 kDa in size, between 20 and 250 kDa in size, between 50 and 250 kDa in size, between 100 and 250 kDa in size, between 150 and 250 kDa in size, between 200 and 250 kDa in size, between 300 and 250 kDa in size, between 400 and 250 kDa in size, between 1 and 200 kDa in size, between 5 and 200 kDa in size, between 10 and 200 kDa in size, between 20 and 200 kDa in size, between 50 and 200 kDa in size, between 100 and 200 kDa in size, between 150 and 200 kDa in size, between 5 and 180 kDa in size, between 5 and 160 kDa in size, between 5 and 150 kDa in size, between 5 and 125 kDa in size, between 5 and 120 kDa in size, between 5 and 100 kDa in size, between 5 and 90 kDa in size, between 5 and 80 kDa in size, between 5 and 75 kDa in size, between 5 and 60 kDa in size, between 5 and 50 kDa in size, between 5 and 45 kDa in size, between 5 and 40 kDa in size, between 5 and 30 kDa in size, between 5 and 20 kDa in size, between 5 and 10 kDa in size, between 10 and 200 kDa, between 10 and 180 kDa in size, between 10 and 160 kDa in size, between 10 and 150 kDa in size, between 10 and 125 kDa in size, between 10 and 120 kDa in size, between 10 and 100 kDa in size, between 10 and 90 kDa in size, between 10 and 80 kDa in size, between 10 and 75 kDa in size, between 10 and 60 kDa in size, between 10 and 50 kDa in size, between 10 and 45 kDa in size, between 10 and 40 kDa in size, between 10 and 30 kDa in size, between 10 and 20 kDa in size, between 20 and 200 kDa, between 20 and 180 kDa in size, between 20 and 160 kDa in size, between 20 and 150 kDa in size, between 20 and 125 kDa in size, between 20 and 120 kDa in size, between 20 and 100 kDa in size, between 20 and 90 kDa in size, between 20 and 80 kDa in size, between 20 and 75 kDa in size, between 20 and 60 kDa in size, between 20 and 50 kDa in size, between 20 and 45 kDa in size, between 20 and 40 kDa in size, between 20 and 30 kDa in size, between 30 and 200 kDa, between 30 and 180 kDa in size, between 30 and 160 kDa in size, between 30 and 150 kDa in size, between 30 and 125 kDa in size, between 30 and 120 kDa in size, between 30 and 100 kDa in size, between 30 and 90 kDa in size, between 30 and 80 kDa in size, between 30 and 75 kDa in size, between 30 and 60 kDa in size, between 30 and 50 kDa in size, between 30 and 45 kDa in size, between 30 and 40 kDa in size, between 40 and 200 kDa, between 40 and 180 kDa in size, between 40 and 160 kDa in size, between 40 and 150 kDa in size, between 40 and 125 kDa in size, between 40 and 120 kDa in size, between 40 and 100 kDa in size, between 40 and 90 kDa in size, between 40 and 80 kDa in size, between 40 and 75 kDa in size, between 40 and 60 kDa in size, between 40 and 50 kDa in size, between 40 and 45 kDa in size, between 50 and 200 kDa in size, between 50 and 180 kDa in size, between 50 and 160 kDa in size, between 50 and 150 kDa in size, between 50 and 125 kDa in size, between 50 and 120 kDa in size, between 50 and 100 kDa in size, between 50 and 90 kDa in size, between 50 and 80 kDa in size, between 50 and 75 kDa in size, between 50 and 60 kDa in size, between 75 and 200 kDa in size, between 75 and 180 kDa in size, between 75 and 160 kDa in size, between 75 and 150 kDa in size, between 75 and 125 kDa in size, between 75 and 120 kDa in size, between 75 and 100 kDa in size, between 75 and 90 kDa in size, between 75 and 80 kDa in size, between 100 and 200 kDa in size, between 100 and 120 kDa, between 100 and 125 kDa in size, between 100 and 150 kDa in size, between 100 and 175 kDa in size, between 125 and 200 kDa in size, between 150 and 175 kDa in size, or between 100 and 175 kDa in size.

In some embodiments, the central nervous system therapeutic agent can be, e.g., a biologic agent (e.g., an enzyme, an antibody, a polypeptide), a sugar, and/or a small molecule. In some embodiments, the CNS therapeutic agent is a therapeutic antibody or fragment thereof. Any suitable therapeutic antibody can be delivered to the CNS using the methods described herein.

In some embodiments, the therapeutic agent is an agent that does not normally cross the blood-CNS barrier. In some embodiments, the CNS therapeutic agent is an agent that inefficiently crosses the blood-CNS barrier, e.g., a therapeutically effective dose of the agent is unable to cross the blood-CNS barrier when administered systemically. In some embodiments, the CNS therapeutic agent is an agent that does efficiently cross the blood-CNS barrier, e.g., a therapeutically effective dose of the agent is able to cross the blood-CNS barrier when administered systemically. Administration of an inhibitor of Ccdc141 signaling at the blood-CNS barrier can increase the permeability of the blood-CNS barrier such that, e.g., a therapeutically effective dose of the CNS therapeutic agent is able to reach the CNS, or the necessary dose of the CNS therapeutic agent is lowered.

Delivery of an inhibitor of Ccdc141 signaling at the blood-CNS barrier to a mammalian subject (e.g., human) may be by, for example, injection to the CNS. In some embodiments, the injection is direct injection to the CNS (e.g., intracerebral injection, intraventricular injection, intracisternal injection, intraparenchymal injection, intrathecal injection, and any combination of the foregoing). In some embodiments, the injection is systemic injection (e.g., intravenous injection, intradermal injection, or subcutaneous injection). In some embodiments, the inhibitor can be administered orally.

II. Decreasing blood-CNS Barrier Permeability

In some aspects, the present disclosure also provides methods for decreasing blood-CNS barrier permeability (i.e., maintaining blood-CNS integrity) in a subject. In some embodiments, the method comprises administering to the subject an agent that promotes Ccdc141 signaling. In some embodiments, the method comprises administering to the subject an agent that inhibits Pacsin2 signaling at the blood-CNS barrier. In some embodiments, an agent that promotes the Ccdc141 signaling decreases expression and/or activity of Pacsin2.

In some embodiments, an agent that promotes the Ccdc141 signaling at the blood-CNS barrier is a recombinant Ccdc141 or a fragment thereof. In some embodiments, an agent that promotes the Ccdc141 signaling at the blood-CNS barrier is a recombinant human Ccdc141 or a fragment thereof. In some embodiments, the recombinant human Ccdc141 comprises an amino acid sequence. The human Ccdc141 amino acid sequence has been described in NM_001316745. An exemplary human Ccdc141 amino acid sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% identical to the amino acid sequence as set forth in SEQ ID NO: 3 (NP_001303674.1). In some embodiments, an exemplary human Ccdc141 amino acid sequence is set forth in SEQ ID NO: 3 (NP_001303674.1):

MSSQGSPSVALSTTTVSSVAVQAGDSKIVIAVIKCGKWVQLQLAESQPNL
LEIGSSQDETKKLLHDHELLLAKLKALEDRVWELLQEADKTAEENKDQSQ
VYDAMAETLGEAWAALVSMLERRTELLRLTSEFFENALEFAIKIDQAEDF
LQNTHEFESAESLKSLLQLHEHHTKELLERSLALLNKSQQLTDFIEKFKC
EGPNVNPELTQGAHSSCLKVDRLLELLQDRRRQLDKYLKQQWQELSQVLQ
ICQWDQQENQVTCWFQKTIRNLQEQSLGSSLSDNEDRIHKQEELIIKAKE
WNSAVEKLKSEALRILLSKDYVEKEHLQLSHQKLSQLQEEFGQLMVERNT
WLKKANEFFNSANKAFDVLGRVEAYLKLLKSEGLSLAVLAVRHEELHRKI
KDCTTDALQKGQTLISQVDSCSSQVSGIHEMMGCIKRRVDHLTEQCSAHK
EYALKKQQLTASVEGYLRKVEMSIQKISPVLSNAMDVGSTRSESEKILNK
YLELDIQAKETSHELEAAAKTMMEKNEFVSDEMVSLSSKARWLAEELNLF
GQSIDYRSQVLQTYVAFLKSSEEVEMQFQSLKEFYETEIPQKEQDDAKAK
HCSDSAEKQWQLFLKKSFITQDLGLEFLNLINMVRMALNGSHLLQGYSSK
SK

In some embodiments, an agent that promotes the Ccdc141 signaling at the blood-CNS barrier is a recombinant mouse Ccdc141 or a fragment thereof. In some embodiments, the recombinant mouse Ccdc141 comprises an amino acid sequence An exemplary mouse Cdc141 amino acid sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% identical to the amino acid sequence as set forth in SEQ ID NO: 4 (NP_001020747.2). In some embodiments, an exemplary mouse Ccdc141 amino acid sequence is set forth in SEQ ID NO: 4 (NP_001020747.2):

MSCKESPHVGASTTTVSSVAVHAGDSKIVIAVVKCGKWVRLQLAESQPNLLEIGSSQDETKK
LLHDHELLLAKLKALEDRVWELLREADRTAEANKAQSQVYDAMAQTLGEAWATLVSMLERRR
ELLGLTSEFFQSALEFAIKIDQAEAFLQNPHEFESTEALQSLLLLHDRHAKELLERSLDLLN
KSQQLTDFIEKFKCEGSTMNSELIQGAQSSCLKIDSLLELLQDRRRQLDKYLQQQRQELSQV
LQLCLWDQQENQVSSWFQKAIRDLQEQSLGASLSDNRELICKHEDLIVKAKEWDSAVEKLKS
QALGILLSKDLAGKEHLQLSNQKLNRLQEEFGRLMVERKAWLSMANDFFTSANKSFDVLGKV
EAYLKLLKSEGLSLPVLAAKHEELHREIKDSTATALQKGRTLISQVDSCRSRVTGIHEMMGY
IQNRVDCLTEQCTAHEEFARKRQQLATSVDDYLRKVEMSIQEIRPILATTLDVASSPSESEK
ILNKYLELDIQVKETAHALEAAAKIMTEKNELELNEVALLPLKVKWLEEELSTLGRSISCRS
RILQTYVAFRKSSEEAEEQLQSLKEFYLTEIPWKDEDDAVVKCQSNSAERKWQLFLKKSFLT
QDLSLEFLNLINMAKENEILNVKNEMHIMENIMEKQTNGREELSHLRVAWYLKAIEGKPARE
QWEMFKEKLTKTTHSVKLLHEVLMPVSALDLGGSLQSTSDLRRRWIAMKPQLQQLHEDVQQI
TKEWEVLSSQGAPLKEKAEQLKDLVHLHRRQRERIQEYEEILYKTVQFHQVKEELVHLIKPR
ELELLAQPMELASSEEVQMQLGRSQERRAHVDHLHQLALTLGVDIISSVQQPNCSNISAKNL
QQQLEALELESRSWSAQAKEHERVLSCSLEYCTARDEISELKESFKDIKKKFNNLKFNYSKK
NEKSRNLKTLQYQIQQVDTYAEKIQALRKKMEKVNNKTSDSFLSYPSNKANMLSEAMEDLKK
NVDDFDKVVTDYKMNLDLTEHLQEVIEECNFWYEDASATVVRVGKYSMECQTREAVDILHRQ
FNKFITPSVPQQEERIQEVIDLAQRLYGLEEGQKYAEKIVTRHKEILESITELCGSLVELKE
KLMQGEVPKMNSDLEDFHDNCIDLLKGPGKDDQKTFSEERNEGQVQGADVLAVNGTREDGLP
MDLRRTSSDKEDSAQGLILPEDTLSGEESECISSDDISLPPLPVSPESPLAPSDMEVEELTS
SSALALHISGYRMHPGTGGLGKAQESALPSPIAFADGGHSKKDTFTSHFERPYPQLKAESSL
ASRGSAEMSTKLHINVKCPASMPHEVHDKALQQCSQARESTLEMQEKVHADSNVTKTQDRLH
AALDVSPGLGSQPDTSESHQRRVGPQGNKKNSSAENSVVSLAGQAPHFSRLLSNVTVMEGSP
VTLEVEVTGFPEPTLTWFKKGQKLCADGHLQVLHKDTKHSVFIPKVCEADAGLYVAQAQNSS
GTLSSKAILHVTGNHGPPITRLNWIMLCIIYVSVSVIYWLLTR

In some embodiments, an agent that promotes the Ccdc141 signaling at the blood-CNS barrier is a nucleic acid encoding Ccdc141. In some embodiments, the nucleic acid encoding Ccdc141 encodes a mammalian Ccdc141 (e.g., human Ccdc141, mouse Ccdc141, rat Ccdc141, non-human primate Ccdc141, et cetera). In some embodiments, the nucleic acid encoding Ccdc141 encodes a human Ccdc141. In some embodiments, the nucleic acid encoding Ccdc141 encodes a mouse Ccdc141.

Human Ccdc141 mRNA sequence is set forth in SEQ ID NO: 1 (NM_000638). In some embodiments, the nucleic acid encoding human Ccdc141 comprises a nucleic acid sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% identical to the nucleotide sequence of SEQ ID NO: 1. The nucleotide sequence encoding human Ccdc 141 includes NM_001316745. An exemplary nucleotide sequence encoding human Ccdc141 protein is set forth in SEQ ID NO: 1 (NM_001316745.2):

GCTCCTTTCTCTCTCACGATAAACTCAATGGGGTTGTCTATTTTTAACCTCCTGTTCTTTCC
TTTTGGCAGAGTAATCAGTGTGAGCTGAGCTCTTTCCCTGAATGAATCCAAGTAACCCTGGC
CTTCTGAAATGACCAGCAGACACACAACTGTCCAGAGGCTGCCCAAAGTATAAACTCTGGTT
CTAAAGTACCATGTCCAGCCAAGGAAGTCCTAGTGTTGCGCTTTCTACGACGACAGTCAGTT
CAGTTGCTGTGCAGGCTGGGGACTCCAAAATCGTTATAGCTGTCATAAAGTGTGGCAAATGG
GTACAACTTCAACTGGCTGAATCACAGCCCAATCTTCTAGAAATTGGCAGCAGTCAAGATGA
AACCAAAAAACTTCTTCATGATCATGAACTTCTTTTGGCCAAGCTCAAGGCTTTGGAAGATC
GGGTATGGGAACTCTTGCAGGAAGCAGACAAGACAGCTGAAGAGAACAAGGATCAGAGTCAG
GTCTATGATGCCATGGCCGAGACTCTGGGTGAAGCATGGGCAGCTCTGGTGTCCATGCTTGA
AAGAAGAACAGAGCTCCTTAGGTTGACTTCTGAATTTTTTGAAAATGCCTTAGAGTTTGCTA
TTAAAATAGACCAAGCTGAAGATTTCCTCCAGAATACTCATGAGTTTGAGAGTGCTGAGTCC
TTAAAATCACTTCTTCAGCTTCATGAACATCATACTAAAGAACTCTTGGAACGGTCTTTAGC
CCTTTTAAACAAAAGTCAACAACTCACTGACTTCATAGAAAAATTCAAGTGTGAAGGACCTA
ATGTGAATCCTGAGTTGACTCAGGGAGCTCATAGCAGCTGTCTGAAGGTTGACCGCCTTCTT
GAACTTCTACAAGACAGGAGAAGACAACTAGACAAGTACTTGAAGCAACAGTGGCAAGAATT
GAGTCAAGTTCTGCAGATATGTCAGTGGGACCAACAAGAAAACCAGGTTACTTGTTGGTTTC
AGAAAACTATAAGAAATTTACAGGAACAAAGTCTAGGTTCATCACTTTCAGACAATGAGGAT
CGAATTCATAAGCAAGAGGAACTGATAATAAAAGCAAAGGAATGGAATTCTGCTGTTGAGAA
GCTGAAGAGTGAGGCACTGAGAATTCTGCTGTCAAAGGACTACGTGGAGAAAGAACACCTCC
AGCTCTCTCACCAGAAACTCAGTCAGCTTCAAGAAGAATTTGGTCAACTCATGGTGGAAAGG
AATACCTGGTTAAAGAAGGCGAATGAATTTTTTAACAGTGCTAACAAGGCATTTGATGTACT
TGGAAGAGTTGAAGCTTACCTTAAGCTCCTTAAATCAGAGGGTTTAAGTCTGGCTGTTTTGG
CAGTGAGGCATGAGGAATTACACAGAAAAATTAAAGACTGCACAACTGATGCTTTGCAAAAG
GGACAAACCTTAATCAGCCAAGTAGACTCCTGCAGCTCTCAGGTGTCCGGCATCCATGAGAT
GATGGGGTGCATTAAGAGACGAGTGGATCATCTGACCGAACAGTGTTCAGCGCACAAGGAAT
ATGCTCTTAAGAAACAACAACTAACAGCCTCAGTGGAGGGTTACCTACGGAAGGTGGAAATG
TCAATTCAGAAAATCAGTCCAGTACTTTCTAATGCAATGGATGTTGGTTCTACCCGTTCTGA
ATCAGAGAAGATTTTGAATAAATATCTGGAACTAGATATCCAAGCTAAGGAGACATCACATG
AATTAGAAGCAGCTGCAAAAACCATGATGGAGAAAAATGAATTTGTATCTGATGAAATGGTA
TCACTTTCCTCTAAAGCTAGATGGCTAGCAGAAGAATTAAACCTATTTGGCCAAAGCATTGA
CTATAGATCGCAAGTCCTGCAAACTTACGTGGCATTTCTGAAGTCATCAGAGGAGGTAGAGA
TGCAGTTTCAGAGCTTAAAAGAATTTTATGAAACCGAAATCCCTCAGAAGGAGCAGGATGAT
GCTAAAGCCAAGCATTGTTCTGACTCGGCTGAGAAGCAGTGGCAGCTATTTTTAAAGAAGAG
TTTTATAACACAAGATCTAGGGCTTGAGTTCCTTAATTTAATAAATATGGTAAGAATGGCAT
TAAATGGATCACACCTTCTGCAGGGATACTCATCAAAGTCAAAATAATAATTTCAAAGGTTC
AGCTGTCTATTAATCAGCAGTTCTCTCCTAGGGAGTTCTCTTTAGCTTCATTCAACCTAGAC
CAGTCCTGAAGCCCAACATTTAAATGCTTGCTTGGCCTAGAACTTTGGTTAGTTTATAACAG
GAAGGCTGTTTTAACTATTAAGAAGAAAGTGACTGTAATCCAAATGCAACATTGTTATTATT
AAATAGAGTCCTCAGAAGTCCACCCAAAAGTTCTGTGTTATTTTGAAAGCATGCAACAAGGT
CCTTTCAAAATTTCTAGGGGCTAAAGACAACTTCTAAGATGAATCTTCTTTTGAAGAGTTTC
TTACCATAGCAGATCTCTTTGTAGTGAAGATCCATTGCATTTAGAAAAATACCCTAACCTTT
AACTGTCTTGAATTATATCTGTAAAATATTTTAGAATCCTTATATCCACCCATAACCCATAT
AAAATTCCCTGTAGGATACTTAAAAGATAGTCCACTTCTCTTTCAATACCTCAAGTATTTTC
CTAATGAAACTGCTCATTTGATTGTTGGAGAATGCCAGCTGCTAGAAACCTTCTTTTTTTAA
TGTTAAATATATCTGTTTCCGTATCTACTCCTAATCATCTGTAGCTACACAGGGACAAAAAG
TATTCATATTTAATTTCTCTTACACATGAAAGTCCTTCCAACATTAAAAACAGTTGCTATGT
CTGAGTTTTTTCTGCAGCATTAAAAATCCTTTCCATCATTTATGAAGGAGCTTTATTTTTCT
TTCTATGCTGATCCCCCCTTCTCAGGATTTTCTATGGTTTGTCAAGATCATTCATTAAATGT
AACCAAAAAGTGAACTACAGGTGCAACACAGAGTACAGTAAACTGGACATTATTATTCACTA
ATGAGACCTACAATTATGCTAACATTTTTAGCAGTAGTTTCACTTTGCTGGTACATATTAAG
CTTGTGATAGAACAAAACTCTCATAATATTTTTATTGCAGCTTCTGTCAAACCATGTCTCTC
CCATATTATACCTGTGTCAGCCATTTACCTTTTTTATACAAATGTGGGCCTTTATATTTTTT
CTTATTTAATTTTATTTTGTTAGTCTTCACACATGATCCCATCTTGCTAAACTAATTTGAAC
CTACCTAGTATATTAGCCATCTGTCTTGGGTTTAATTCTGTAGATATGACAAGTAAGCTTTC
TATGGCAGCAATTAGTTCTTAAATATAAATGCCTAAAGGGCAGATTGGCATTCCAGTTCATG
AAGCAGTCTATTGATTAAACCAGTTTTTAAAATAATCATGAATGCTCCCAATAGTACTTCAT
TCAGCCGCTACTTCATAATCTTTGTATTAAGTCTATAATGGGAGACTTTCTCAAATAAACTG
CTCAAAAGAAGGTTTACCTACCTAGTATCTTATCAATGACATAGACAATTTGACATAAATCA
GGTTGGCTCCTAGCGATCAAGGCATTCTTTCCAAAATGCTCACAAAGTGCTTGATAAGTAAT
GAACTTATTGCACTGATTTTCAGTGTGAAGCTTACCAATCTGTAAATTGTGTAGTCCATCCT
TTCCTCCTTTTGAAAATCAGGGCATTTTCTGTTCTCTAATTTTATGGCTCTTCTAGTTTCTG
TAGATTTTCAAAGATAACTGACAGCTGCAATTTTCTCTGTACCTGGGATGTGATTAATCTCA
GTCTGGAGCCTGAATTCATTTGGAAGAGTTAGGTTCTAACTACCCTCATCTACTTGGAGCTG
CAATTTTCTCTTTATCTTGTTTCAACTTTTGTTGTTTGAAGACCATTCTTGATAAACCTACT
TGGAAATAAAATATGCATTAGCTGATTCTCATTTATTCAACATGATTCTTAAGCACTAAAGA
AAACTTGCTAAGCAATTTGCCCATGTTCCTATGTGATCTAGGTGCTTTACTGCAAAAAAAAA
GAAAAGTTATTTTTCGTTTAGAACATTTAGAACATGTATTATCTATCTATCTATCTCTGTCT
CTCTCTCACACTTCTACGTGGTTCAATCAGTCTTCTCCCATCTGTTGATGATGTATGTTTCT
GATTTACCTAAGGGAATTCAAATACTTTCAAACTGCGTAATTTTGTCTACCTTTTTATTACT
AACTTCATTTCAGAAATTTTAGTTCTGGAATCTACAGTCGGTACTTTTATTTATTAAATTAC
TGATGAAATTCTGTTTCTTGTCTGCTCTTTTCCCATCTTTCCAATACTTTCTTGAACTATAT
TAACCCTAGGCACTTAAAGTGTTCATCAGCTAGCTACAACATTTGTACCTGGAGCTCTTTCT
TTCTTTCTTTCCTTTCTTTTCTTTTTTTTTTAGATGGAGTCTCGCTCTGTCACCCAGACTGG
AGTGCAGTGGTGCGATCTCAGCTCCCTTTAACCTCCAGCTCCTGGGTTCAAGCAATTCTCAT
GCCTCAGTCCCCCGATTAGCTGGAATTACAGGTGCCTGCCACCATGCCCGGCTAATACTTGT
ATTTTTAGTAGAGACGAGGTTTCACCATGTTGACCAGGCTGGTCTCAAACTCCTGACTTCAA
GTGATCTGCCCACCTCTGCCTCCCAAAGTGCTGGGATTACAGGCATGAACCACAGCGCCCGG
CCAATCTGGAGCTGTTTCTGTTGTCTAATTTTTCTCTTGGTTTCCAGTCCTGTAGTCCTGTT
TCTTGGCAGAACTAGCGAATATTTATCAAATGCCAGACATTATGTATAAAAAATTGGAGGAT
CAGCAGATGGTGTTTTCTTCTTCTAGAAAGGGTCACCCTTCTTTCTCCAGGCAGGCATTGGT
GGGCAATCACTCAAGCCAATCAGAAGCTGGCAGAGTTGTGTTTGCAGTGTAGTCCTGTCCCC
TCAGGCTCCCAGAGTTTTGTATTGAGAGCCAAGTGCTTTCTCTGACGTCTCTGCCCCTGGCA
GGTCACAAACTCCAATCTTTGTTTGGGTAGTCTGCTGAAAAACTCCACTCTGCTTTTCAGAG
GCCTTTTGCTTAGGTTTTTTAATCCACTGCCCGATATACCCCTGGTATTTTGTCAACGTCTT
TAGAAGATCATTGTGCTTGAAGCCCTCCAAGTGTCTGCCAAAATCTCTGGTGTTTTCTCGGC
TCCAGGGAAGTCTCGATTATCAGCTTCCTGTCTGCACAGAATTGGGAAATCCCCCTGGGATG
ATAGGTGAGCTCACCCCTGTCAGGTTCTTCCCTCTCTGGAGTCAGCCTCTCTAGTTCCGGTT
GTCCGACCAGTTCTCTGCTTCCTTCAAATGGATTCCTTCTGCATTTTACCCAGCTTTTCTAG
GTATTCTAAGCAAAAGCACTGCTCTGCCACTGGCTACCCCATTCTACTCAGAAGTCCTTTGT
TACTTATTTAGAATGTTTAAGATTGTACAGTCAGATTTTTGTTTTTAAAGATCTGCAACAAT
AACTTATTCCAAATTATATTTTGTATCTATATCTAAATAAACTTAGGTTATCATAATATTCA
ATGCCCACTTAGATCTCCCAGAAATAGCATATATTTTACTACTGTGCTTTGGTGTTCTTTTA
TCTTGATGTCATAAACACGGTCACTCTACCCTAAGGTCTTTACCACTATCATATGATAAATA
AGTTCCTCCTTATTGATAAAATTAAAGCCCACGTACTAAGGAAATTTATTGGTTAGCAACAT
TTGGAGAGATGAAATTGTTGGTAAAGTAAGTAAAACGATTTTTTTTATTCTTATATCTTTTG
GACCACTAGGCTTTTAAACAAACATTATTGTTGAATCATGGTGACTAGTTTGCTTGCTAAGA
AGGCAGAGGCCAGCTGGTATATTCAGTTGAGTGTTCTGTTCTTTATTGGGTTGTCGTGGCTC
ATATTTTCAAGCCAGAAATAGAAAAAATTTCAGTTCATCTAAAAATCATTAAGATCAGAGAA
ACTGATTCAGGCCCAAATTATGGCAGACATCTTGATGAAAGTGCTAATTTATTAATTTATAA
TGGCCAATACCAAGAATGCTATCTGGGATACAGCCTTCCTATTATCAAGGTATCTGAAAAGC
AAGACATCAGAAAACCATTCATGGTGCATGAATATAAACGAAAAATGAATGCTTTAGTGGCC
TGTTTAGACTGGATTATGACAAACATTTTCCATGTCCTACACAGAAGTAAAATGAGTACTGT
GTTTACTAATTAGACCTCAGGCAAATTATTCAATCTTCTTGAGTGGCCAAAATATTCGTCTG
CCAAATACATGATCACTTTTCACAGATCCTACTAGCTAGACATCTACAAATCCTACTTTAGC
TCTCCTTTCTCTAGACCTCACTCAGACCACCATATCCTTCTGATTAAAGTTGTCATTGGCAT
GCTGCAGTTTCAAAATTTATATTAAATTATATAATACATTACATTCTGGTTAATCATTTCTT
TAAAATATGTGAAAGATAATTTTATTTATGATAAAAGTATAATTTGGGGTGAATTCTGCTGT
AGTTGTCTAATATCTTTCATAATTTATTATGTATCATTATTAATAATGAGGGAATATAACGT
TTTATTACTAGATTCTTAAGTTCATGGGGGAAATCTCATTTAGGACCTACAGATATATATTT
TTTTGGAGTTGCCACTTTATAATGTGTAAAATAACATAAAAATATAAGTGTGCAACATCAAT
ATCATTTAAAATAATTTTAAAATAAAATGGCCTATCTTTAAAATATCCTGTTAAGTATTTAT
GATACTGTCAATTGCTGTCATTGAGATGATAGAAATTTGAAGATTTATTGAGAAAGCCTGGC
TATTAAAGTAAAAAACATAAGAATTGTTTCGCCAAATGAATGTTCCTGTGCATTTTTTAAAT
GTTTCTACAGAAATAAAGCACGCATAGTCTTA

Mouse Ccdc141 mRNA sequence is set forth in SEQ ID NO: 2 (NM_001025576.3). In some embodiments, the nucleic acid encoding mouse Ccdc141 comprises a nucleic acid sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% identical to the nucleotide sequence of SEQ ID NO: 2. Nucleotide sequences encoding mouse Ccdc141 protein include NM_001025576, XM_619767, XM_886279, XM_896648, XM_896657, XM_896667, XM_896676, XM_896697, XM_896711, XM_896724, XM_912240, XM_922274, XM_922277, XM_922280, XM_922286, XM_922295, XM_922299, XM_922303, XM_922309. An exemplary nucleotide sequence encoding mouse Ccdc141 protein is set forth in SEQ ID NO: 2 (NM_001025576.3):

TGTTCTTTCCTTTTGGCCGGGCAATCAGTGTGAACAGAGGCGCGTTCCCTGAAAGAGTCCCG
GTAGCCTCGTGGAAGCCTTCTGACATGACCAGCAGACAGCATCACTACCAAGGCTGCTCGGG
GTTTAAGCCCTGCTTCTAAAGTGCCATGTCCTGCAAAGAAAGTCCCCATGTGGGTGCTTCCA
CCACCACTGTCAGCTCTGTAGCCGTGCACGCTGGGGACTCCAAGATCGTGATCGCCGTTGTC
AAGTGTGGCAAATGGGTACGACTCCAACTGGCTGAATCGCAGCCCAATCTCCTCGAAATTGG
GAGCAGTCAAGATGAAACCAAAAAACTCCTTCACGATCATGAGCTGCTTCTGGCCAAGCTTA
AGGCCTTGGAAGATCGTGTGTGGGAACTCTTACGGGAAGCAGACCGGACAGCTGAAGCAAAC
AAGGCTCAAAGTCAGGTGTATGACGCCATGGCCCAGACGCTGGGTGAGGCATGGGCAACCCT
GGTCTCCATGCTTGAAAGAAGAAGGGAGCTCCTTGGGCTGACGTCCGAGTTCTTTCAAAGTG
CTTTGGAGTTTGCTATAAAAATAGACCAAGCTGAAGCTTTTCTGCAGAATCCTCATGAGTTT
GAGAGCACTGAAGCCTTACAGTCACTTCTTCTGCTTCATGACCGACATGCCAAAGAACTCTT
AGAAAGATCCCTAGACCTTTTAAACAAAAGTCAACAACTGACTGACTTCATAGAAAAATTCA
AGTGTGAAGGATCTACTATGAATTCTGAGCTGATCCAGGGAGCTCAGAGCAGCTGTCTGAAG
ATCGACAGCCTCCTTGAACTTCTGCAAGACAGGAGAAGGCAGCTCGACAAGTACTTGCAGCA
ACAGCGGCAGGAATTGTCTCAGGTTCTGCAGTTGTGTCTGTGGGACCAACAAGAAAACCAGG
TTTCTTCTTGGTTTCAGAAAGCAATAAGAGATCTGCAGGAACAGAGTCTGGGCGCATCACTT
TCAGACAACAGAGAGCTAATCTGCAAGCACGAGGACCTGATAGTCAAAGCAAAGGAATGGGA
TTCGGCCGTAGAGAAGCTGAAAAGCCAGGCTTTGGGGATTCTATTGTCCAAGGACTTGGCAG
GAAAGGAACATCTACAGCTCTCTAACCAGAAACTGAATCGGCTTCAAGAAGAATTTGGAAGA
CTCATGGTGGAAAGGAAAGCCTGGTTATCGATGGCAAATGACTTTTTTACCAGTGCTAACAA
GTCATTTGATGTACTTGGGAAAGTTGAAGCTTACCTTAAGCTCCTTAAATCAGAGGGTTTAA
GCCTGCCTGTCTTGGCAGCGAAGCATGAGGAATTACACAGAGAAATTAAAGACTCTACAGCC
ACTGCTCTGCAAAAGGGACGAACCTTAATCAGCCAAGTAGACTCCTGCCGCTCTCGGGTGAC
AGGAATCCACGAGATGATGGGATACATTCAGAACCGAGTGGATTGTCTCACTGAACAGTGTA
CAGCGCATGAGGAATTTGCCCGCAAGAGACAGCAATTAGCAACATCAGTGGACGACTACCTA
AGGAAGGTGGAAATGTCAATTCAGGAAATCAGGCCCATTCTTGCTACCACATTGGATGTTGC
TTCCAGCCCTTCTGAATCAGAGAAGATTTTAAATAAATATCTGGAATTGGATATCCAAGTCA
AGGAGACAGCACATGCATTAGAAGCAGCTGCAAAAATCATGACAGAGAAAAATGAACTTGAA
CTCAATGAAGTGGCATTGCTTCCTCTTAAAGTGAAATGGCTAGAGGAAGAATTAAGCACGCT
TGGTCGAAGCATCAGCTGCAGGTCACGGATCCTGCAGACGTACGTGGCATTTCGAAAGTCAT
CCGAGGAGGCAGAGGAGCAGCTTCAGAGCCTAAAGGAATTTTATCTGACTGAAATTCCTTGG
AAAGATGAAGACGATGCTGTGGTCAAGTGCCAGTCTAACTCCGCTGAGAGGAAATGGCAGCT
GTTTTTAAAGAAGAGTTTTTTGACCCAGGACTTGAGCCTCGAGTTTCTGAACTTAATAAACA
TGGCAAAAGAGAATGAGATATTAAATGTTAAAAATGAAATGCACATTATGGAGAACATTATG
GAAAAGCAAACAAATGGAAGGGAAGAGCTCAGCCATCTCCGAGTGGCGTGGTACCTCAAAGC
TATCGAAGGCAAGCCTGCGAGAGAGCAGTGGGAAATGTTCAAAGAGAAACTTACAAAGACTA
CTCACAGTGTAAAACTTCTCCACGAAGTTCTCATGCCTGTCTCTGCGCTTGACCTTGGGGGG
AGCCTCCAGAGCACGTCAGATCTGCGGAGGAGGTGGATCGCAATGAAGCCTCAGCTCCAGCA
ACTGCATGAGGATGTTCAGCAAATCACGAAAGAGTGGGAAGTGTTAAGCAGTCAGGGAGCTC
CTCTGAAGGAGAAGGCTGAACAGCTGAAGGACCTTGTCCATCTCCACCGGAGGCAGCGAGAG
CGAATCCAGGAATATGAGGAAATCCTATACAAGACAGTCCAGTTCCACCAGGTCAAGGAAGA
GCTGGTCCATCTCATCAAACCAAGAGAACTGGAACTTCTAGCACAGCCCATGGAACTGGCAA
GTTCTGAGGAGGTCCAGATGCAACTGGGCCGTTCGCAAGAAAGGCGGGCACATGTAGACCAT
CTCCATCAGCTGGCCCTGACCTTAGGAGTGGACATCATTTCATCCGTGCAACAACCTAATTG
CTCTAATATTTCTGCAAAGAACCTGCAGCAGCAGCTGGAGGCCCTTGAACTGGAGAGCAGGA
GTTGGAGTGCCCAAGCCAAAGAACATGAGCGCGTCCTGTCCTGCAGCTTGGAGTACTGCACT
GCACGTGATGAGATCAGTGAGCTCAAAGAGTCATTCAAAGACATAAAAAAGAAATTCAACAA
TCTGAAGTTTAATTACTCCAAGAAAAATGAGAAATCTCGAAATTTAAAGACTCTTCAGTATC
AAATCCAACAAGTGGATACATATGCTGAAAAAATTCAGGCTCTAAGGAAGAAAATGGAAAAA
GTTAATAATAAGACATCTGATTCATTCTTAAGTTATCCAAGTAATAAAGCTAACATGCTTTC
GGAAGCCATGGAGGATTTGAAAAAGAACGTGGATGACTTTGACAAAGTTGTGACAGATTACA
AAATGAATTTGGACCTGACTGAGCATCTTCAGGAAGTAATAGAAGAGTGTAACTTTTGGTAT
GAAGATGCAAGTGCCACCGTTGTGAGAGTTGGGAAGTATTCCATGGAGTGCCAAACAAGGGA
AGCTGTGGACATCCTCCACAGGCAGTTCAATAAGTTTATTACTCCCTCGGTGCCCCAGCAAG
AAGAAAGGATTCAGGAGGTCATTGACCTTGCTCAGCGCTTATATGGTTTGGAAGAGGGGCAG
AAATATGCAGAGAAGATTGTCACAAGACACAAGGAGATTCTTGAATCTATTACTGAATTATG
TGGGTCTCTCGTGGAACTTAAAGAAAAACTGATGCAGGGAGAGGTTCCAAAGATGAATTCAG
ATTTGGAAGATTTCCATGACAATTGCATTGATCTGCTAAAGGGGCCAGGGAAGGATGACCAG
AAAACATTCAGTGAAGAAAGGAATGAGGGGCAGGTGCAGGGAGCAGATGTATTGGCCGTCAA
TGGAACAAGAGAAGATGGGCTTCCCATGGACTTGAGGCGGACCTCCTCTGACAAAGAGGACA
GCGCCCAGGGTCTGATCCTTCCAGAAGACACACTATCTGGAGAAGAATCTGAGTGTATCTCA
TCCGATGACATCTCTTTGCCTCCACTGCCAGTGAGCCCCGAGTCCCCTCTGGCACCATCAGA
CATGGAGGTAGAAGAACTTACCAGCTCCTCTGCCCTCGCCCTGCACATCAGCGGCTACAGGA
TGCATCCAGGGACAGGTGGTCTAGGAAAGGCCCAGGAGTCTGCTCTTCCCTCACCCATTGCC
TTTGCGGATGGGGGCCATAGTAAGAAAGACACATTTACAAGTCATTTTGAGAGGCCCTATCC
ACAGTTGAAAGCTGAATCCTCATTAGCCTCCAGAGGATCAGCAGAGATGAGTACCAAGTTGC
ACATCAATGTGAAATGTCCAGCGAGCATGCCCCATGAAGTGCACGACAAAGCTTTACAGCAA
TGCTCCCAGGCTCGGGAGAGCACACTAGAAATGCAGGAAAAAGTGCATGCTGATTCTAACGT
CACTAAAACCCAAGACAGGCTGCATGCTGCCCTTGATGTGTCCCCTGGCCTCGGCTCTCAAC
CAGACACCAGCGAGAGCCATCAGAGGCGAGTGGGTCCCCAAGGAAACAAGAAAAACTCATCC
GCAGAAAACAGCGTGGTCAGCCTAGCTGGCCAGGCACCTCATTTCTCCAGGCTGCTGTCCAA
TGTAACTGTCATGGAAGGTTCCCCAGTGACTTTGGAAGTCGAAGTAACAGGATTCCCAGAGC
CTACACTGACATGGTTCAAGAAGGGCCAGAAACTGTGTGCAGATGGACACTTACAGGTCTTG
CACAAGGACACAAAGCATTCGGTGTTCATTCCGAAGGTATGTGAGGCAGATGCTGGCCTCTA
TGTGGCTCAAGCCCAAAACTCTAGTGGCACCCTGTCTTCCAAAGCCATCCTCCATGTGACAG
GTAACCATGGGCCACCAATTACAAGACTAAACTGGATTATGCTGTGTATCATCTATGTCAGT
GTATCAGTAATATACTGGCTACTTACACGATAGTGACATGTTGCCATCAATGGACATTGTTA
TGAGCCTAAAAGACAATATGATTGTTGATTTTCTCATACATCACACAAAGCACTCCCCCAAC
ATACACACACCAATTTATCTCCACTTTGACTAATCCCACTGCTAGGTGAAATAGTTGTATTT
TCTCTACGATTTAACATACTTGGGAAACACGGGAAATTATGTACAGCGGGTAATGATACATT
TCATACTCTAGGAAGAGAAAGATTCTGTCTAATAAGCTGATCTCTTGTTTAAAGGCATATCA
ATGCGCACATAGTTCACAAGGATCATAGGGTTTTGTCCTAATTTTCAAATCCAAATGTTTTT
CATTTCTCCTTCACTGTGCTCATAACTATCTCTTTGTAGGGCATTGCATCAAACTGAGGTTG
TGATTCTCAATGTTGTATTAGCTTCCATTAACTGTGCTTTTCACATTTTGATCTTGTTTACC
AAACAAACTTGACCTATGCTGTCTCAACCTACTCTACATTCCTACGTCGGAGGATGTCACTA
TCAATAACCATTCCTCATGATGGTCTCTTGGGCTTGATGTTGGAGAACAGACGTGCCATCAG
CTTCAAAATGGAGTCAACCTTTCTATTAGCTCTGAATGTCATATGTCAAAATGACATACAGG
CTGCAGGAGGGGACCAGTTTTCCCCCTGTCCTTCCACTGCTGTGCATTGACCATCTGTGCAT
AACTATAGGCAGGTCCTACTGCATCTGCTGCTGCTGCTGTTGCTGCTGAACACTGTGTAGCC
CTTTACAAGGCATCTCAGTGCCCCCCCTTTCCCATCTGGAAGAGGGGCTCATCTACCTCTTG
ACAACTGAGTTTTTATTTTATGTCCTGTGGTGATCTGGCCAATGGAAAAATACTCAGAGAAG
ACTCATTACAATGCTTGGTGTGATTTGCCATCATGCTACTCATTACCCATTAATTCCCTTTC
AGATTCTGATCTTTTAGAATATTAAATGAAAAGAGTGCTGTTAATATGTAATTTCTAACTAC
TCCCTTGGGGGAGGGTAAGACTGATGTTCTATGAATTAGAGTTTAAATTCCTTTCTAACTTC
TTACAACTACATCCTTTTCAAAGGCTCTGGACGGTAACCTCTGGAGGATATAGTTAGCGTGT
TTTAGAGGGAGTCTGGGTAAGATAGAAGAAGACAATTCTAGCTTTCTTTTGTGCAATTATCT
CTGTAATATTGAAGAATGTTGTATGGCTTCCTAATACGAATTATTAAAAAAAATAAGAGAGG
TGTTTGTTTTCATATTTTAAACTTTGTCATGCTAGGGTAAAATCAAGTTGAAAGAATAATTT
ATTACAGATTTTTGATGAAGGGAAGAAATTAATTATGTTGCTGAAAGACTGTCTACAAATAA
TCAAAATATTTAAATAGAATATATTTGATGTGATATTAAAAAGCTACTGAATGTTGTGGAAG
ATAAGAAATGTCTAGTCTAGTAAAAAATTGGCCTATTTTTTCTTGTCCTGTGTATACAATTT
AATTGAAAAATTGTAACTGTTTTCTTTCAAGTTTTGACAGAAAGCTATTAAAATCTTATATA
TACAC

In some embodiments, the nucleic acids encoding a Ccdc141 can be administered to the subject using any suitable known method, for example, but not limited to, direct injection, viral vector mediated delivery (e.g., AAV, retrovirus, adeno virus, or lentivirus), or ceDNA. In some embodiments, an agent that promotes Ccdc141 expression/activity is an rAAV encoding Ccdc141.

The present disclosure, at least in part, is based on the surprising discovery that inhibition of Pacsin2 in CNS endothelial cells results in an increase in blood-CNS barrier permeability (i.e., maintaining blood-CNS integrity) through decreased tubular vesicle-mediated transcytosis. In some aspects, the method comprises administering to the subject a Pacsin2 inhibitor. Pacsin2 protein is involved in linking the actin cytoskeleton with vesicle formation by regulating tubulin polymerization and mediates vesicle or tubule formation for endocytosis across brain endothelial cells (Ritter et al., PACSIN 2, a novel member of the PACSIN family of cytoplasmic adapter proteins, FEBS Letters (1999) 454(9): 356-362; Leite et al., The Role of BAR Proteins and the Glycocalyx in Brain Endothelium Transcytosis, Cells (2020), 9(12): 2685).

In some embodiments, the method comprises administering to a subject an inhibitor of Pacsin2 signaling. In some embodiments, the Pacsin2 is expressed by CNS endothelial cells. In some embodiments, the method comprises administering to the subject an agent that inhibits Pacsin2 signaling at the blood-CNS barrier.

In some embodiments, the inhibitor of Pacsin2 signaling is a Pacsin2 inhibitor. In some embodiments, inhibition of Pacsin2 in CNS endothelial cells results in decreased blood-CNS barrier permeability. In some embodiments, inhibition of Pacsin2 does not result in decreased blood-CNS barrier permeability.

In some embodiments, the Pacsin2 inhibitor is capable of inhibiting Pacsin2 expression and/or activity. In some embodiments, the Pacsin2 inhibitor is an inhibitory nucleic acid targeting Pacsin2 mRNA. As described herein, an inhibitory nucleic acid, refers to nucleic acids capable of inhibiting expression or activity of a target gene (e.g., DNA, RNA, or protein of the target gene), for example, PACSIN2. Non-limiting examples of inhibitory nucleic acids include e.g., dsRNA, siRNA, shRNA, miRNA, amiRNA, antisense oligonucleotides (ASOs), DNA or RNA aptamers, etc.

In some embodiments, an inhibitory nucleic acid targeting Pacsin2 is an siRNA. siRNA molecules comprise a specific antisense sequence in addition to the reverse complement (sense) sequence.

The specificity of siRNA molecules may be determined by the binding of the antisense strand of the molecule to its target RNA (e.g., Pacsin2 mRNA). In some embodiments, the siRNA molecules are 60, 65, 70, 75, 80, 85, 90, 95, 100 or more base pairs in length. In some embodiments, the antisense sequence of the siRNA molecules is 7, 8, 9, 10, 11, 12, 13, 14, 15, 16, 17, 18, 19, 20, 21, 22, 23, 24, 25, 26, 27, 28, 29, 30, 35, 40, 45, 50, or more base pairs in length. In some embodiments, the antisense sequence of the siRNA molecules are 8 to 30 base pairs in length, 10 to 15 base pairs in length, 10 to 20 base pairs in length, 15 to 25 base pairs in length, 19 to 21 base pairs in length, or 21 to 23 base pairs in length.

In some embodiments, siRNA molecules comprise an antisense sequence comprising a region of complementarity to a target region in a Pacsin2 mRNA (e.g., human Pacsin2 mRNA (SEQ ID NO: 5) or mouse Pacsin2 mRNA (SEQ ID NO: 6)). In some embodiments, the region of complementarity is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to a target region in a Pacsin2 mRNA (e.g., human Pacsin2 mRNA or mouse Pacsin2 mRNA). In some embodiments, the target region is a region of consecutive nucleotides in a Pacsin2 mRNA (e.g., human Pacsin2 mRNA or Pacsin2 Ccdc141 mRNA). In some embodiments, a complementary nucleotide sequence need not be 100% complementary to that of its target to be specifically hybridizable or specific for a Pacsin2 mRNA (e.g., human Pacsin2 mRNA or mouse Pacsin2 mRNA). Exemplary human Pacsin2 mRNA and mouse Pacsin2 mRNA sequence is set forth in SEQ ID NO: 5 and SEQ ID NO: 6.

In some embodiments, siRNA molecules comprise an antisense sequence that comprises a region of complementarity to in a Pacsin2 mRNA (e.g., human Pacsin2 mRNA or mouse Pacsin2 mRNA) sequence and the region of complementarity is in the range of 8 to 15, 8 to 30, 8 to 40, or 10 to 50, or 5 to 50, or 5 to 40 nucleotides in length. In some embodiments, the region of complementarity is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary to at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a Pacsin2 mRNA (e.g., human Pacsin2 mRNA (SEQ ID NO: 5) or mouse Pacsin2 mRNA (SEQ ID NO: 6)). In some embodiments, the region of complementarity comprises a nucleotide sequence that contains no more than 1, 2, 3, 4, or 5 base mismatches compared to the complementary portion of a Pacsin2 mRNA (e.g., human Pacsin2 mRNA or mouse Pacsin2 mRNA). In some embodiments, the region of complementarity comprises a nucleotide sequence that has up to 3 mismatches over 15 bases, up to 2 mismatches over 10 bases, or up to 1 mismatch over 5 bases.

In some embodiments, siRNA molecules targeting Pacsin2 comprise an antisense strand which comprises a region of complementarity that is at least 60%, at least 65%, at least 70%, at least 75%, at least 80%, at least 85%, at least 90%, at least 95%, or 100% complementary to the sequences as set forth in any one of SEQ ID NOs: 22-31, and 36-45. In some embodiments, siRNA molecules targeting Pacsin2 comprise an antisense strand which comprises a region of complementarity that is complementary to at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of the sequences as set forth in any one of SEQ ID NOs: 22-31, and 36-45.

Exemplary Pacsin2 siRNAs target sequences are set forth below:

Pacsin2 target sequences:
(SEQ ID NO: 22)
CACATATGATGATTCCGTTGGAGTA
(SEQ ID NO: 23)
TCGGGAACTACAAGCGGACTGTGAA
(SEQ ID NO: 24)
CAAGAACTGGCAGAAGGAAGCCTTT
(SEQ ID NO: 25)
GAAAGAGGTAGAAGCAGCAAAGAAA
(SEQ ID NO: 26)
CAAAGAGGAGAAGCTGGCTATCTCA
(SEQ ID NO: 27)
CAAGCAAGATGTTCTTAAGACCAAA
(SEQ ID NO: 28)
TAGACCTGTCCAATGTGGCTGGCTA
(SEQ ID NO: 29)
CCTGTCCAATGTGGCTGGCTACAAA
(SEQ ID NO: 30)
CAGCTACGAGAAGACCCAGAGCTAT
(SEQ ID NO: 31)
AGTTGGCCTATACCCGGCAAATTAT
(SEQ ID NO: 36)
GGCCTCACTGATGAACGATGA
(SEQ ID NO: 37)
GGTAGAAGCAGCAAAGAAAGC
(SEQ ID NO: 38)
GCAAGCAAGATGTTCTTAAGA
(SEQ ID NO: 39)
GCACCTAGACCTGTCCAATGT
(SEQ ID NO: 40)
GGCTGGCTACAAAGCCATTTA
(SEQ ID NO: 41)
GCTGGCTACAAAGCCATTTAC
(SEQ ID NO: 42)
GCTACAAAGCCATTTACCATG
(SEQ ID NO: 43)
GGTCAGACGATGAGTCTAACA
(SEQ ID NO: 44)
GGCCTATACCCGGCAAATTAT
(SEQ ID NO: 45)
GCCTATACCCGGCAAATTATG

In some embodiments, the present disclosure also contemplates delivering inhibitory nucleic acids targeting Pacsin2 mRNA that are known in the art, for example, CAAAUUAUGUGGAGGCGAU (SEQ ID NO: 32), CCCUUAAUGUCCCGAGCAA (SEQ ID NO: 33), CCUCACUGAUGAACGAUGA (SEQ ID NO: 34), or CUGAGGUGGUUCCGAGCCA (SEQ ID NO: 35) as described by Hansen et a1. (Pacsin2 is recruited to caveolae and functions in caveolar biogenesis, J Cell Sci (2011) 124 (16): 2777-2785), or sc-36174 (m), and sc-36173 (h) from Santa Cruz Biotechnology.

In some embodiments, an inhibitory nucleic acid targeting Pacsin2 is an shRNA.

The specificity of shRNA molecules may be determined by the binding of the antisense strand of the molecule to its target RNA sequence (e.g., Pacsin2 mRNA; SEQ ID NO: 5 or EQ ID NO: 6). In some embodiments, the shRNA molecules are 60, 65, 70, 75, 80, 85, 90, 95, 100 or more base pairs in length. In some embodiments, the antisense sequence of the shRNA molecules is 7, 8, 9, 10, 11, 12, 13, 14, 15, 16, 17, 18, 19, 20, 21, 22, 23, 24, 25, 26, 27, 28, 29, 30, 35, 40, 45, 50, or more base pairs in length. In some embodiments, the antisense sequence of the shRNA molecules are 8 to 30 base pairs in length, 10 to 15 base pairs in length, 10 to 20 base pairs in length, 15 to 25 base pairs in length, 19 to 21 base pairs in length, or 21 to 23 base pairs in length.

Following selection of an appropriate target RNA sequence, shRNA molecules that comprise a nucleotide sequence complementary to all or a portion of the target sequence, i.e., an antisense sequence, can be designed and prepared using methods known in the art (see, e.g., Moore et al., Short Hairpin RNA (shRNA): Design, Delivery, and Assessment of Gene Knockdown, Methods Mol Biol. 2010; 629:141-158).

The shRNA molecules can comprise a hairpin (i.e., when two regions of the same strand, usually complementary in nucleotide sequence when read in opposite directions, base-pair to form a double helix that ends in an unpaired loop), or asymmetric hairpin (i.e., hairpin with a strand overhang) secondary structure, having self-complementary sense and antisense strands. In some embodiments, the shRNA targeting inhibitory nucleic acid described herein comprises an antisense sequence and a sense sequence.

In some embodiments, the antisense sequence of the shRNA molecule is 7, 8, 9, 10, 11, 12, 13, 14, 15, 16, 17, 18, 19, 20, 21, 22, 23, 24, 25, 26, 27, 28, 29, 30, 35, 40, 45, 50, or more nucleotides in length. In some embodiments, the antisense sequence is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 19 to 21 nucleotides in length, or 21 to 23 nucleotides in lengths.

In some embodiments, the sense sequence of the shRNA molecule is 7, 8, 9, 10, 11, 12, 13, 14, 15, 16, 17, 18, 19, 20, 21, 22, 23, 24, 25, 26, 27, 28, 29, 30, 35, 40, 45, 50, or more nucleotides in length. In some embodiments, the sense sequence is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 19 to 21 nucleotides in length, or 21 to 23 nucleotides in lengths.

In some embodiments, shRNA molecules comprise an antisense sequence comprising a region of complementarity to a target region in a Pacsin2 mRNA (e.g., human Pacsin2 mRNA or mouse Pacsin2 mRNA). In some embodiments, the region of complementarity is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the target region in a Pacsin2 mRNA (e.g., human Pacsin2 mRNA or mouse Pacsin2 mRNA) of SEQ ID NOs: 22-31 and 36-45. In some embodiments, the target region is a region of consecutive nucleotides in a Pacsin2 mRNA (e.g., human Pacsin2 mRNA or mouse Pacsin2 mRNA). In some embodiments, a complementary nucleotide sequence need not be 100% complementary to that of its target to be specifically hybridizable or specific for a Pacsin2 mRNA (e.g., human Pacsin2 mRNA or mouse Pacsin2 mRNA).

In some embodiments, shRNA molecules comprise an antisense sequence that comprises a region of complementarity in a Pacsin2 mRNA (e.g., human Pacsin2 mRNA or mouse Pacsin2 mRNA) sequence and the region of complementarity is in the range of 8 to 15, 8 to 30, 8 to 40, or 10 to 50, or 5 to 50, or 5 to 40 nucleotides in length. In some embodiments, the region of complementarity is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary to at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a Pacsin2 mRNA (e.g., human Pacsin2 mRNA or mouse Pacsin2 mRNA). In some embodiments, the region of complementarity comprises a nucleotide sequence that contains no more than 1, 2, 3, 4, or 5 base mismatches compared to the complementary portion of a Pacsin2 mRNA (e.g., human Pacsin2 mRNA or mouse Pacsin2 mRNA). In some embodiments, the region of complementarity comprises a nucleotide sequence that has up to 3 mismatches over 15 bases, up to 2 mismatches over 10 bases, or up to 1 mismatch over 5 bases.

In some embodiments, shRNA molecules target a Pacsin2 sequence comprises an antisense strand that comprises a region of complementarity that is at least 60%, at least 65%, at least 70%, at least 75%, at least 80%, at least 85%, at least 90%, at least 95%, or 100% complementary to the sequences as set forth in any one of SEQ ID NOs: 22-31, and 36-45. In some embodiments, shRNA molecules targeting Pacsin2 comprise an antisense strand that is complementary to at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of the sequence as set forth in any one of SEQ ID NOs: 22-31 and 36-45.

In some embodiments, the inhibitory nucleic acids targeting Pacsin2 can be administered to the subject using any suitable known method, for example, but not limited to, direct injection, viral vector mediated delivery (e.g., AAV, retrovirus, adenovirus, or lentivirus), or ceDNA.

In some embodiments, the Pacsin2 inhibitor is an antibody, an antibody variant or an antigen-binding fragment thereof targeting Pacsin2. Antibodies, antibody variants and antigen-binding fragments targeting Pacsin2 have been previously described, see, e.g., ab228589, ab262841 from Abcam, aa400-450 from CSBio, PA5-83983, PA5-84299, MA5-37727, PA5-99031, PA5-118137 or PA1-41564 from Invitrogen.

In some embodiments, administration of an effective amount of any one of the Pacsin2 inhibitors or an agent that promotes Ccdc 141 expression/activity described herein or a combination thereof at the blood-CNS barrier results in decreased blood-CNS barrier permeability.

An effective amount of an agent that decreases blood-CNS barrier permeability (e.g., a Pacsin2 inhibitor or an agent that promotes Ccdc141 expression/activity) described herein may be an amount sufficient to have a therapeutic benefit in a subject, e.g., to extend the lifespan of a subject, to improve and/or reverse in the subject one or more symptoms of disease, or to slow disease progression. The effective amount will depend on a variety of factors such as, for example, the species, age, weight, health of the subject, and the tissue to be targeted, and may thus vary among subject and tissue. An effective amount may also depend on the inhibitor used.

In some embodiments, administration of an agent that decreases blood-CNS barrier permeability (e.g., a Pacsin2 inhibitor or an agent that promotes Ccdc141 expression/activity) described herein may result in inhibition of Pacsin2 signaling and/or increase of Ccdc141 signaling of one or more of the CNS endothelial cells. In some embodiments, administration of the inhibitors described herein may result in inhibition of Pacsin2 signaling and/or Ccdc141 signaling in all of the foregoing endothelial cells.

An effective amount may also depend on the mode of administration. For example, targeting endothelial cells in the CNS by intravenous administration or subcutaneous injection may require different (e.g., higher, or lower) doses, in some cases, than targeting endothelial cells in the CNS by another method (e.g., local injection to the CNS). In some embodiments, a Pacsin2 inhibitor described herein is a small molecule and can be administered orally.

In some embodiments, administering an inhibitor of Pacsin2 signaling at the blood-CNS Barrier decreases the level and/or activity of Pacsin2. As used herein, administration of the inhibitor described herein decreases the expression and/or activity of Pacsin2 by at least 10% or more, e.g., by 10% or more, 50% or more, 100% or more, 200% or more, 500% or more, or 1000% or more. In some embodiments, administration of the Pacsin2 inhibitors described herein results in decreasing of the permeability of the blood-CNS barrier in the subject. In some embodiments, administration of the Pacsin2 inhibitor described herein results in decreasing the permeability of the blood-CNS barrier in the subject by at least 10%, at least 15%, at least 20%, at least 25%, at least 30%, at least 35%, at least 40%, at least 45%, at least 50%, at least 55%, at least 60%, at least 65%, at least 70%, at least 80%, at least 85%, at least 90%, at least 95%, at least 99%, at least 100%, at least 150%, at least 200%, at least 300%, at least 400%, at least 500% or more compared the permeability of the blood-CNS barrier of the subject prior to administration of the inhibitor. The permeability of blood-CNS barrier can be measured using any suitable technique or method known in the art, e.g., neuroimaging techniques including dynamic perfusion CT (PCT) and dynamic contrast-enhanced magnetic resonance imaging (DCEMRI), quantification of protein biomarkers (e.g., neuron-specific enolase (NSE), glial fibrillary acidic protein (GFAP), and S100β) in the cerebral spinal fluid (CSF), etc.

In some embodiments, administering an agent that promotes Ccdc141 signaling at the blood-CNS Barrier increases the level and/or activity of Ccdc141. As used herein, administration of an agent that promotes Ccdc141 signaling described herein increases the expression and/or activity of Ccdc141 by at least 10% or more, e.g., by 10% or more, 50% or more, 100% or more, 200% or more, 500% or more, or 1000% or more. In some embodiments, administration of an agent that promotes Ccdc141 signaling described herein results in decreasing of the permeability of the blood-CNS barrier in the subject. In some embodiments, administration of an agent that promotes Ccdc141 signaling described herein results in decreasing the permeability of the blood-CNS barrier in the subject by at least 10%, at least 15%, at least 20%, at least 25%, at least 30%, at least 35%, at least 40%, at least 45%, at least 50%, at least 55%, at least 60%, at least 65%, at least 70%, at least 80%, at least 85%, at least 90%, at least 95%, at least 99%, at least 100%, at least 150%, at least 200%, at least 300%, at least 400%, at least 500% or more compared the permeability of the blood-CNS barrier of the subject prior to administration of the inhibitor. The permeability of blood-CNS barrier can be measured using any suitable technique or method known in the art, e.g., neuroimaging techniques including dynamic perfusion CT (PCT) and dynamic contrast-enhanced magnetic resonance imaging (DCEMRI), quantification of protein biomarkers (e.g., neuron-specific enolase (NSE), glial fibrillary acidic protein (GFAP), and S100β) in the cerebral spinal fluid (CSF), etc.

Without wishing to be bound by any particular theory, efficient delivery of an agent that promotes Ccdc141 signaling or inhibits Pacsin2 signaling at the blood-CNS barrier may be useful for the treatment of a subject having a disease associated with endothelial cell dysfunction (e.g., head trauma, stroke, et cetera). In certain embodiments, the endothelial cells are endothelial cells in the CNS. Accordingly, methods and compositions for treating diseases associated with endothelial cell dysfunction are also provided herein. In some aspects, the disclosure provides a method for treating a disease associated with endothelial cell dysfunction, the method comprising: administering to a subject having or suspected of having a disease associated with endothelial cell dysfunction an effective amount of an agent that promotes Ccdc141 signaling or inhibits Pacsin2 signaling at the blood-CNS barrier.

Endothelial cells (e.g., endothelial cells in the CNS) can be healthy endothelial cells (e.g., endothelial cells in the CNS not having a dysfunction, or at risk of developing endothelial cell dysfunction), or dysfunctional endothelial cells (e.g., endothelial cells causing abnormal vasculature permeability, hemodynamics, or neuroimmune crosstalk, et cetera). As used herein, “or at risk of developing endothelial cell dysfunction” refers to a subject having an increased probability of developing endothelial cell dysfunction than the general population due to the presence of a risk factor. Exemplary categories of risk factors for developing endothelial cell dysfunction include, but are not limited to, genetics, head trauma, vascular disease, prior brain surgery, disease, age, race, and family history (e.g., positive family history of vascular disease, high cholesterol, high blood pressure, or diabetes). As used herein, an “disease associated with endothelial cell dysfunction” is a disease or condition that results from the dysfunction of endothelial cells (e.g., endothelial cells in the CNS). In some embodiments, a disease associated with CNS endothelial cell dysfunction includes but is not limited to, retinal disease (e.g., diabetic retinopathy), neurodegenerative disease (e.g., Huntington's disease, dementia), acute injury of the CNS (e.g., stroke and head trauma), Neuroinfectious disease (e.g., encephalitis, sepsis, COVID-19), primary and metastatic cancers of the CNS, autoimmune disease of the CNS (e.g., multiple sclerosis), and other neuroinflammatory conditions. In some embodiments, the disease associated with CNS endothelial cell dysfunction is a CNS primary cancer, such as glioblastoma, meningioma, or lymphoma. In some embodiments, the disease associated with CNS endothelial cell dysfunction is a metastatic cancer to the brain such as metastatic lung cancer, metastatic breast cancer, or melanoma. In some embodiments, the disease associated with CNS endothelial cell dysfunction is a neuroinflammatory disease, such as CNS Lupus, CNS Lyme Disease, Neurosarcoidosis, Neuromyelitis optica (NMO), or Paraneoplastic and Autoimmune Encephalitis. In some embodiments, the disease associated with CNS endothelial cell dysfunction is a dementia and/or cognitive disorder, such as dementia resulting from Alzheimer's disease, Lewy body dementia, frontotemporal dementia, encephalopathy, or post-acute COVID syndrome.

Ccdc141 inhibits tubular vesicle transcytosis that is mediated by Pacsin2. Ccdc141 gene ablation or Pacsin2 gene overexpression in the brain endothelial cells in mice leads to an accumulation of tracer-filled tubular vesicles (e.g., tubular vesicles are involved in transcytosis (i.e., vesicular trafficking) of molecules across the blood-brain barrier endothelial cell layer, from blood into the brain tissue). Analogous tracer-filled tubular vesicles have been observed in the disrupted blood-brain barrier vasculature in the brain and spinal cord, under several neuropathological conditions. Specifically, tubular vesicles have been observed in the brain endothelial cells after traumatic brain injuries suggesting Pacsin2 overexpression in these conditions (see., e.g., Lossinsky et al., New ultrastructural evidence for a protein transport system in endothelial cells of gerbil brains. Acta Neuropathol 47, 105-110; Lossinsky et al., Ultracytochemical studies of vesicular and canalicular transport structures in the injured mammalian blood-brain barrier. Acta Neuropathol 61, 239-245; Lossinsky et al., Ultracytochemical evidence for endothelial channel-lysosome connections in mouse brain following blood-brain barrier changes. Acta Neuropathol 53, 197-202; Lossinsky et al., A comparative ultrastructural study of endothelial cell tubular structures from injured mouse blood-brain barrier and normal hepatic sinusoids demonstrated after perfusion fixation with osmium tetroxide. Microvasc Res 31, 333-344, doi:10.1016/0026-2862 (86) 90022-1 (1986)) and stroke (see, e.g., Tagami, M. et a1. Increased transendothelial channel transport of cerebral capillary endothelium in stroke-prone SHR. Stroke14, 591-596, doi: 10.1161/01.str.14.4.591 (1983)) and in the spinal cord endothelial cells in the EAE mouse and rat models for multiple sclerosis (see, e.g., Claudio et al., Increased vesicular transport and decreased mitochondrial content in blood-brain barrier endothelial cells during experimental autoimmune encephalomyelitis. Am J Pathol 135, 1157-1168 (1989); Lossinsky et al., Sites of egress of inflammatory cells and horseradish peroxidase transport across the blood-brain barrier in a murine model of chronic relapsing experimental allergic encephalomyelitis. Acta Neuropathol 78, 359-371. In other embodiments, Pacsin2 is overexpressed in ageing brain where blood-CNS barrier permeability has been shown to be increased (e.g., early onset dementia such as frontotemporal dementia (see, Gerrits et al., Neurovascular dysfunction in GRN-associated frontotemporal dementia identified by single-nucleus RNA sequencing of human cerebral cortex, Nat Neurosci. 2022 August; 25(8):1034-1048). In some embodiments, Ccdc141 is decreased in conditions where blood-CNS barrier permeability have been shown to be increased (e.g., stoke (see, Garcia-Bonilla et al., Brain and blood single-cell transcriptomics in acute and subacute phases after experimental stroke; bioRxiv. Preprint. 2023 Apr. 3), spinal cord injury (see, Cao et al., Single-cell RNA sequencing for traumatic spinal cord injury, The FASEB Journal. 2022; 36:e22656), ageing (see, Ximerakis et al., Single-cell transcriptomic profiling of the aging mouse brain, Nature Neuroscience volume 22, pages 1696-1708 (2019)). Accordingly, in some embodiments, the present disclosure contemplates treating diseases and conditions associated with increased blood-CNS permeability (e.g., ageing, early onset dementia, stroke, or traumatic spinal cord injury) by administering a subject in need thereof an agent that inhibits Pacsin2 expression/activity and/or promotes Ccdc141 expression/activity. In some embodiments, the present disclosure provides methods of inhibiting transcytosis at the blood-CNS barrier by administering a subject in need thereof an agent that inhibits Pacsin2 expression/activity and/or promotes Ccdc141 expression/activity.

In some embodiments, an agent that promotes Ccdc141 signaling or inhibits Pacsin2 signaling at the blood-CNS can be administered to a subject in need of improved integrity (e.g., decreased permeability) of the blood-CNS barrier. In some embodiments, the subject in need of improved quality of vesicle trafficking in the blood-CNS barrier can be a subject who has been diagnosed with or determined to have abnormally high permeability of the blood-brain barrier, e.g., repeated infections of the CNS, or in which abnormal levels of a systemically administered tracer molecule reach the CNS. In some embodiments, the subject having abnormally high blood-CNS barrier permeability described herein has an increasing of the permeability of the blood-CNS barrier compared to a healthy subject. In some embodiments, the subject having abnormally high blood-CNS barrier permeability described herein has an increasing of the permeability of the blood-CNS barrier by at least 10%, at least 15%, at least 20%, at least 25%, at least 30%, at least 35%, at least 40%, at least 45%, at least 50%, at least 55%, at least 60%, at least 65%, at least 70%, at least 80%, at least 85%, at least 90%, at least 95%, at least 99%, at least 100%, at least 150%, at least 200%, at least 300%, at least 400%, at least 500% or more compared the permeability of the blood-CNS barrier of a healthy subject. The permeability of blood-CNS barrier can be measured using any suitable technique or method known in the art, e.g., neuroimaging techniques including dynamic perfusion CT (PCT) and dynamic contrast-enhanced magnetic resonance imaging (DCEMRI), quantification of protein biomarkers (e.g., neuron-specific enolase (NSE), glial fibrillary acidic protein (GFAP), and S100β) in the cerebral spinal fluid (CSF), etc.

In some embodiments, the subject in need of improved quality of vesicle trafficking of the blood-brain barrier can be a subject in need of treatment (e.g. having, diagnosed as having, or at risk of developing) a condition selected from the group consisting of dementia, encephalitis, sepsis, COVID-19, primary and metastatic cancers of the CNS, autoimmune disease of the CNS (e.g., multiple sclerosis), and other neuroinflammatory conditions. In some embodiments, the disease is a CNS primary cancer, such as glioblastoma, meningioma, or lymphoma. In some embodiments, the disease is a metastatic cancer to the brain such as metastatic lung cancer, metastatic breast cancer, or melanoma. In some embodiments, the disease is a neuroinflammatory disease, such as CNS Lupus, CNS Lyme Disease, Neurosarcoidosis, Neuromyelitis optica (NMO), or Paraneoplastic and Autoimmune Encephalitis. In some embodiments, the disease is a dementia and/or cognitive disorder, such as dementia resulting from Alzheimer's disease, Lewy body dementia, frontotemporal dementia, encephalopathy, or post-acute COVID syndrome.

In some embodiments, administration of an agent that promotes Ccdc141 signaling or inhibits Pacsin2 signaling at the blood-CNS barrier can slow or halt the progression of a neurodegenerative disease. In some embodiments, administration of an agent that promotes the Ccdc141 signaling at the blood-CNS barrier can slow or prevent the development of at least some signs or symptoms of any of the diseases described herein.

Delivery of an agent that promotes Ccdc141 signaling at the blood-CNS barrier or inhibits Pacsin2 signaling in a mammalian subject may be by, for example, injection to the CNS. In some embodiments, the injection is direct injection to the CNS (e.g., intracerebral injection, intraventricular injection, intracisternal injection, intraparenchymal injection, intrathecal injection, and any combination of the foregoing). In some embodiments, the injection is systemic injection (e.g., intravenous injection, intradermal injection, or subcutaneous injection). In some embodiments, an agent that promotes Ccdc141 signaling or inhibits Pacsin2 signaling at the blood-CNS barrier can be administered orally.

III. Delivery of Nucleic Acids Using rAAV

The present disclosure provides compositions and methods for delivering a transgene to endothelial cells throughout the CNS in a subject. In some aspects, the disclosure provides isolated and/or engineered AAVs. In some aspects, the present disclosure provides a recombinant adeno-associated virus (rAAV), wherein the rAAV comprises: (i) an AAV capsid protein (e.g., AAV-BI30, AAV-BR1, AAV9 or variants thereof), and (ii) an isolated nucleic acid encoding an agent for modulating blood-CNS barrier as described herein (e.g., an agent increasing or decreasing Ccdc141 and/or Pacsin2 expression/activity).

As used herein with respect to AAVs, the term “isolated” refers to an AAV that has been artificially produced or obtained. Isolated AAVs may be produced using recombinant methods. Such AAVs are referred to herein as “recombinant AAVs”. Recombinant AAVs (rAAVs) preferably have tissue-specific targeting capabilities, such that a transgene (e.g., a transgene encoding Ccdc141 and/or Pacsin2 or an inhibitory nucleic acid targeting Ccdc141 and/or Pacsin2) of the rAAV will be delivered specifically to one or more predetermined tissue(s) or cell(s) (e.g., endothelial cells in the CNS).

The term “transgene,” as used herein, refers to a gene that has been transferred from one organism to another by any known suitable genetic engineering techniques. The introduction of a transgene has the potential to change the phenotype of an organism. Transgene describes a segment of DNA containing a gene sequence that has been isolated from a first organism and is introduced into a second organism. This non-native segment of DNA may either retain the ability to produce RNA or protein in the transgenic organism or alter the normal function of the transgenic organism's genetic code. In some embodiments, the transgene encodes a Ccdc141 and/or Pacsin2 protein. In some embodiments, the transgene encodes an inhibitory nucleic acid targeting Ccdc141 and/or Pacsin2.

The AAV capsid is an important element in determining tissue-specific targeting capabilities of the virus. Thus, a rAAV having a capsid appropriate for the tissue being targeted can be selected. In some embodiments, the target tissue/cells of the present disclosure are endothelial cells in the CNS (e.g., brain arterial endothelial cells, brain venous endothelial cells, brain capillary endothelial cells, endothelial cells of the spinal cord, or endothelial cells of the retina).

Methods for obtaining recombinant AAVs having a desired capsid protein are well known in the art. See, for example, US Patent Application Publication, US-2003-0138772, the contents of which are incorporated herein by reference. Typically the methods involve culturing a host cell which contains a nucleic acid sequence encoding an AAV capsid protein; a functional rep gene; a recombinant AAV vector comprising AAV inverted terminal repeats (ITRs) and an isolated nucleic acid comprising a transgene (e.g., a transgene for expressing a Ccdc141 and/or Pacsin2 protein or an inhibitory nucleic acid targeting Ccdc141 and/or Pacsin2); and sufficient helper functions to permit packaging of the recombinant AAV vector into the AAV capsid.

In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are about 87 kDa, about 72 kDa, and about 62 kDa, respectively. In some embodiments, upon translation, capsid proteins form a spherical 60-mer protein shell around the viral genome. In some embodiments, the functions of the capsid proteins are to protect the viral genome, deliver the genome, and interact with the host. In some aspects, capsid proteins deliver the viral genome to a host in a tissue-specific or cell-specific manner. In some embodiments, an AAV capsid protein is of an AAV serotype selected from the group consisting of AAV-BI30 (see, e.g., WO2023004367), AAV-9, AAV9.PHP.B, AAV9.PHP.eB, or AAV-BR1 (see, e.g., WO 2015158749). In some embodiments, the AAV capsid protein is AAV-BI30. In some embodiments, the AAV capsid protein is AAV-BR1. In some embodiments, the capsid protein is of AAV serotype 9 (AAV9). In some embodiments, an AAV capsid protein is of a serotype derived from AAV9 (e.g., an AAV9 capsid variant). In some embodiments, the AAV9 capsid variant is AAV9.PHP.B.

The isolated nucleic acids of the invention may be recombinant adeno-associated virus (AAV) vectors (rAAV vectors). In some embodiments, an isolated nucleic acid as described by the disclosure comprises two adeno-associated virus (AAV) inverted terminal repeats (ITR) flanking the transgene. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, a transgene and its regulatory sequences (e.g., a promoter), and 5′ and 3′ AAV inverted terminal repeats (ITRs). The transgene may comprise, as disclosed elsewhere herein, a nucleotide sequence encoding a Ccdc141 and/or Pacsin2 protein or an inhibitory nucleic acid targeting Ccdc141 and/or Pacsin2. In some embodiments, an rAAV encoding a Ccdc141 protein comprises the nucleic acid sequence of SEQ ID NOs: 1 or 2. In some embodiments, an rAAV encoding a Pacsin2 protein comprises the nucleic acid sequence of SEQ ID NOs: 5 or 6. In some embodiments, an rAAV encoding an inhibitory nucleic acid targeting Pacsin2 comprises the nucleic acid sequence of SEQ ID NOs: 9-21. In some embodiments, an rAAV encoding an inhibitory nucleic acid targeting Ccdc141 comprises the nucleic acid sequence of SEQ ID NOs: 22-31, and 36-45

In some embodiments, the transgene (e.g., a transgene encoding a Ccdc141 and/or Pacsin2 protein or an inhibitory nucleic acid targeting Ccdc141 and/or Pacsin2) may further comprise a promoter operably linked to the coding sequence (e.g., nucleotide sequence encoding the Ccdc141 and/or Pacsin2 protein or the inhibitory nucleic acid targeting Ccdc141 and/or Pacsin2). A “promoter” refers to a DNA sequence recognized by the synthetic machinery of the cell, or introduced synthetic machinery, required to initiate the specific transcription of a gene. The phrases “operatively linked,” “under control,” or “under transcriptional control” means that the promoter is in the correct location and orientation in relation to the nucleic acid to control RNA polymerase initiation and expression of the gene. A promoter may be a constitutive promoter, inducible promoter, or a tissue-specific promoter.

As used herein, a nucleotide sequence (e.g., a nucleotide sequence encoding Ccdc141 and/or Pacsin2 protein or an inhibitory nucleic acid targeting Ccdc141 and/or Pacsin2) and regulatory sequences are said to be operably linked when they are covalently linked in such a way as to place the expression or transcription of the nucleic acid sequence encoding Ccdc141 and/or Pacsin2 protein or an inhibitory nucleic acid targeting Ccdc141 and/or Pacsin2 under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence, and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide.

In some embodiments, the promoter is a constitutive promoter. Examples of constitutive promoters include, without limitation, the retroviral Rous sarcoma virus (RSV) LTR promoter (optionally with the RSV enhancer), the cytomegalovirus (CMV) promoter (optionally with the CMV enhancer) (see, e.g., Boshart et al., Cell, 41:521-530 (1985)), the SV40 promoter, the dihydrofolate reductase promoter, the β-actin promoter, the phosphoglycerol kinase (PGK) promoter, and the EF1-α promoter (Invitrogen). In some embodiments, the promoter is hybrid cytomegalovirus (CMV) immediate-early/Chicken beta-actin promoter (CAG promoter). In some embodiments, the promoter is a chicken beta-actin (CBA) promoter. In some embodiments, the promoter is a minimal promoter. A minimal promoter is a part of a promoter located between-35 to +35 region with respect to the transcription start site. It has one or more of 3 conservative sequences, i.e., Tata box, initiator region, binding site for RNA polymerase, and downstream promoter element. Exemplary minimal promoters can be less than 400, 400, 200, 195, 190, 185, 180, or less nucleotides in length. In some embodiments, the promoter us a U6 promoter.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech, and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter for the transgene is used. The native promoter may be preferred when native expression of the transgene is desired. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites, or Kozak consensus sequences, may also be used to mimic the native expression. In some embodiments, the promoter is a native promoter (e.g., Ccdc141 and/or Pacsin2 promoter). In some examples, the promoter can drive the transgene expression (e.g., Ccdc141 and/or Pacsin2 protein or inhibitory nucleic acid targeting Ccdc141 and/or Pacsin2) in endothelial cells in the CNS.

In some embodiments, the regulatory sequences impart tissue-specific gene expression capabilities. In some cases, the tissue-specific regulatory sequences bind tissue-specific transcription factors that induce transcription in a tissue-specific manner. Such tissue-specific regulatory sequences (e.g., promoters, enhancers, etc.) are well known in the art. In some embodiments, the tissue-specific promoter is an endothelial cell-specific promoter.

The 5′ untranslated region (5′ UTR) (also known as a leader sequence or leader RNA) is the region of an mRNA that is directly upstream from the initiation codon. The 5′ UTR plays important roles in both transcriptional and translational regulation of the downstream gene (e.g., the Ccdc141 and/or Pacsin2 gene). In some embodiments, a transgene (e.g., transgene for expressing a Ccdc141 and/or Pacsin2 protein) comprises a nucleotide encoding a 5′ UTR.

The presence of an intron or intervening sequence in mRNA was first described, in vitro, to be important for mRNA processing and increased transgene expression (e.g., Huang Z M, Yen T S. Role of the hepatitis B virus posttranscriptional regulatory element in export of intron less transcripts. Mol Cell Biol. 1995; 15(7):3864-3869; Niwa M, Rose S D, Berget S M. In vitro polyadenylation is stimulated by the presence of an upstream intron. Genes Dev. 1990; 4(9): 1552-1559, which are incorporated herein by reference). In some embodiments, an isolated nucleic acid described herein may also contain an artificial intron, desirably located between the promoter/enhancer sequence and the nucleotide sequence encoding a Ccdc141 and/or Pacsin2 protein or an inhibitory nucleic acid targeting Ccdc141 and/or Pacsin2). In some embodiments, an intron is a synthetic or artificial (e.g., heterologous) intron. Examples of synthetic introns include an intron sequence derived from SV-40 (referred to as the SV-40 T intron sequence) and intron sequences derived from the chicken beta-actin gene. In some embodiments, a transgene described by the disclosure comprises one or more (1, 2, 3, 4, 5, or more) artificial introns. In some embodiments, the one or more artificial introns are positioned between a promoter and a nucleotide sequence encoding the transgene. In some embodiments, the isolated nucleic acid comprises a chimeric intron.

In some embodiments, the transgene (e.g., the transgene for expressing a Ccdc141 and/or Pacsin2 protein or an inhibitory nucleic acid targeting Ccdc141 and/or Pacsin2) expression cassette further comprises a nucleotide sequence encoding a 3′UTR located 3′ of the nucleotide sequence encoding the Ccdc141 and/or Pacsin2 protein or an inhibitory nucleic acid targeting Ccdc141 and/or Pacsin2. In some embodiments, the 3′ UTR is Ccdc141 and/or Pacsin2 gene 3′ UTR.

In some embodiments, the transgene comprises a 3′-untranslated region (3′-UTR). In some embodiments, the disclosure relates to isolated nucleic acids comprising a transgene encoding a Ccdc141 and/or Pacsin2 protein or an inhibitory nucleic acid targeting Ccdc141 and/or Pacsin2, and one or more miRNA binding sites. Without wishing to be bound by any particular theory, incorporation of miRNA binding sites into gene expression constructs allows for regulation of transgene expression (e.g., inhibition of transgene expression) in cells and tissues where the corresponding miRNA is expressed. In some embodiments, incorporation of one or more miRNA binding sites into a transgene allows for de-targeting of transgene expression in a cell-type specific manner. In some embodiments, one or more miRNA binding sites are positioned in the 3′ untranslated region (3′ UTR) of a transgene, for example between the last codon of a nucleic acid sequence encoding a Ccdc141 and/or Pacsin2 protein or an inhibitory nucleic acid targeting Ccdc141 and/or Pacsin2, and a poly A sequence.

The methods typically involve administering to a subject an effective amount of a rAAV encoding an agent (e.g., inhibitory nucleic acid inhibiting Ccdc141 or Pacsin2, or nucleic acid encoding Ccdc141 or Pacsin2) for expressing a transgene in the subject to increase or decrease BBB permeability. Additional embodiments involve administering to a subject an effective amount of a rAAV comprising a nucleic acid encoding a transgene in the subject to increase or decrease BBB permeability. In some embodiments, the transgene of the present disclosure encodes Pacsin2 (e.g., human Pacsin2 or mouse Pacsin2). In some embodiments, the transgene of the present disclosure encodes Ccdc141 (e.g., human Ccdc141 or mouse Ccdc141). In some embodiments, the transgene of the present disclosure encodes an inhibitory nucleic acid targeting Ccdc141 (e.g., siRNA or shRNA targeting Ccdc141 as described herein). In some embodiments, the transgene of the present disclosure encodes an antibody targeting Ccdc141. In some embodiments, the transgene of the present disclosure encodes an inhibitory nucleic acid targeting Pacsin2 (e.g., siRNA or shRNA targeting Pacsin2 as described herein). In some embodiments, the transgene of the present disclosure encodes an antibody targeting Pacsin2. An “effective amount” of a rAAV is an amount sufficient to infect a sufficient number of cells of target cells in a subject. In some embodiments, the target cells of the rAAV are endothelial cell of the CNS. In some embodiments, the endothelial cells of the CNS are brain endothelial cells. In some embodiments, the brain endothelial cells are arterial endothelial cells, venous endothelial cells, and capillary endothelial cells. In some embodiments, the brain capillary endothelial cells are brain microvascular endothelial cells (BMVECs). In some embodiments, the endothelial cells of the CNS are spinal cord endothelial cells. In some embodiments, the endothelial cells of the CNS are retina vasculature endothelial cells. In some embodiments, the retina vasculature endothelial cells are superficial plexus arterial endothelial cells, superficial plexus venous endothelial cells, intermediate plexus endothelial cells, or deep plexus endothelial cells. An effective amount of a rAAV may be an amount sufficient to have a therapeutic benefit in a subject, e.g., to extend the lifespan of a subject, to improve and/or reverse in the subject one or more symptoms of disease, or to slow disease progression. The effective amount will depend on a variety of factors such as, for example, the species, age, weight, health of the subject, and the tissue to be targeted, and may thus vary among subject and tissue.

An effective amount may also depend on the rAAV used. The invention is based, in part on the recognition that rAAV comprising capsid proteins having a particular serotype (e.g., an AAV9 capsid protein variant such as AAV-BI30 or an AAV2 capsid protein variant such as AAV-BR1) mediate more efficient transduction of endothelial cells throughout the CNS than a rAAV comprising capsid proteins having a different serotype. In some embodiments, the rAAV comprises a capsid protein of an AAV serotype of AAV-BI30. In some embodiments, the rAAV comprises a capsid protein of an AAV serotype of AAV-BR1.

In some embodiments, AAV-BI30 or AAV-BR1 has tropism for endothelial cells. In some embodiments, AAV-BI30 has tropism for endothelial cells in the central nervous system (CNS). In some embodiments, the endothelial cells of the CNS are brain endothelial cells. In some embodiments, AAV-BI30 has tropism for brain arterial endothelial cells, brain venous endothelial cells, and brain capillary endothelial cells. In some embodiments, AAV-BI30 has tropism for brain microvascular endothelial cells (BMVECs). In some embodiments, AAV-BI30 has tropism for spinal cord endothelial cells. In some embodiments AAV-BI30 has tropism for retina vasculature endothelial cells. In some embodiments, AAV-BI30 has tropism for retina endothelial cells such as superficial plexus arterial endothelial cells, superficial plexus venous endothelial cells, intermediate plexus endothelial cells, or deep plexus endothelial cells. In some embodiments, AAV-BI30 has tropism for peripheral endothelial cells, such as endothelial cells in the lung (e.g., lung microvasculature endothelial cells), endothelial cells in the aorta, or endothelial cells in the interlobular vessels of the kidney. In some embodiments, AAV-BI30 shows stronger tropism toward endothelial cells in the CNS than endothelial cells in peripheral tissues as measured by any suitable method in the art (e.g., transduce higher percentage of endothelial cells in the CNS and/or with higher transduction efficiency compared to endothelial cells in peripheral tissues). In some embodiments, administration of the rAAV or a composition thereof described herein may result in transduction of one or more of the foregoing endothelial cells. In some embodiments, administration of the rAAV or a composition thereof described herein may result in transduction of all of the foregoing endothelial cells. In some embodiments, administration of the rAAV or a composition thereof described herein may result in transduction of the endothelial cells throughout the CNS, but not the endothelial cells in peripheral tissues.

In certain embodiments, the effective amount of rAAV is 10¹⁰, 10¹¹, 10¹², 10¹³, or 10¹⁴ genome copies per kg. In certain embodiments, the effective amount of rAAV is 10¹⁰, 10¹¹, 10¹², 10¹³, 10¹⁴, or 10¹⁵ genome copies per subject.

An effective amount may also depend on the mode of administration. For example, targeting endothelial cells in the CNS by intravenous administration or subcutaneous injection may require different (e.g., higher or lower) doses, in some cases, than targeting endothelial cells in the CNS by another method (e.g., local injection to the CNS). The disclosure is based, in part, on the recognition that intravenous injection (i.v.) of rAAV having certain serotypes (e.g., AAV-BI30) mediates efficient transduction of endothelial cells in the CNS. Thus, in some embodiments, the injection is intravenous injection (i.v.). In some embodiments, single dose of a rAAV is administered. In some cases, multiple doses of a rAAV are administered.

Without wishing to be bound by any particular theory, efficient transduction of endothelial cells in the CNS by rAAV described herein may be useful for the treatment of a subject having a disease associated with endothelial cell dysfunction (e.g., head trauma). Accordingly, methods and compositions for treating disease associated with endothelial cell dysfunction are also provided herein. In some aspects, the disclosure provides a method for treating a disease associated with endothelial cell dysfunction, the method comprising: administering to a subject having or suspected of having a disease associated with endothelial cell dysfunction an effective amount of rAAV, wherein the rAAV comprises (i) an AAV-BI30 capsid protein or an AAV-BR1 capsid protein; and (ii) a nucleic acid comprising a promoter operably linked to a transgene.

Delivery of the rAAVs to a mammalian subject may be by, for example, injection to the CNS. In some embodiments, the injection is direct injection to the CNS (e.g., intracerebral injection, intraventricular injection, intracisternal injection, intraparenchymal injection, intrathecal injection, and any combination of the foregoing). In some embodiments, the injection is systemic injection (e.g., intravenous injection, intradermal injection, or subcutaneous injection).

In some embodiments, a composition further comprises a pharmaceutically acceptable carrier. Suitable carriers may be readily selected by one of skill in the art in view of the indication for which the rAAV is directed. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover. For example, one acceptable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

The rAAV containing pharmaceutical composition disclosed herein may further comprise a suitable buffer agent, include, but are not limited to, HEPES (4-(2-hydroxyethyl)-1-piperazineethanesulfonic acid) buffer, Dulbecco's phosphate-buffered saline (DPBS) buffer, or Phosphate-buffered Saline (PBS) buffer. Such buffers may comprise disodium hydrogen phosphate and sodium chloride, or potassium dihydrogen phosphate and potassium chloride.

Optionally, the compositions of the disclosure may contain, in addition to the rAAV and carrier(s), other pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The rAAV containing pharmaceutical composition described herein comprises one or more suitable surface-active agents, such as a surfactant. Surfactants are compounds that lower the surface tension (or interfacial tension) between two liquids, between a gas and a liquid, or between a liquid and a solid. Surfactants may act as detergents, wetting agents, emulsifiers, foaming agents, and dispersants. Suitable surfactants include, in particular, non-ionic agents, such as polyoxyethylenesorbitans (e.g., Tween™ 20, 40, 60, 80, or 85) and other sorbitans (e.g., Span™ 20, 40, 60, 80, or 85). Compositions with a surface-active agent will conveniently comprise between 0.05 and 5% surface-active agent, and can be between 0.1 and 2.5%. It will be appreciated that other ingredients may be added, for example, mannitol or other pharmaceutically acceptable vehicles, if necessary.

The rAAVs are administered in sufficient amounts to transduce the cells of a desired tissue (e.g., endothelial cells in the CNS) and to provide sufficient levels of gene transfer and expression without undue adverse effects. Examples of pharmaceutically acceptable routes of administration include, but are not limited to, direct delivery to the selected organ (e.g., CNS), intravenous, intramuscular, subcutaneous, intradermal, and other parental routes of administration. Routes of administration may be combined, if desired.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors.

In some embodiments, a dose of rAAV is administered to a subject no more than once per calendar day (e.g., a 24-hour period). In some embodiments, a dose of rAAV is administered to a subject no more than once per 2, 3, 4, 5, 6, or 7 calendar days. In some embodiments, a dose of rAAV is administered to a subject no more than once per calendar week (e.g., 7 calendar days). In some embodiments, a dose of rAAV is administered to a subject no more than bi-weekly (e.g., once in a two-calendar week period). In some embodiments, a dose of rAAV is administered to a subject no more than once per calendar month (e.g., once in 30 calendar days). In some embodiments, a dose of rAAV is administered to a subject no more than once per six calendar months. In some embodiments, a dose of rAAV is administered to a subject no more than once per calendar year (e.g., 365 days or 366 days in a leap year). In some embodiments, a dose of rAAV is administered to a subject once.

### IV. Kits and Related Composition

The agents described herein may, in some embodiments, be assembled into pharmaceutical or research kits to facilitate their use in therapeutic, or research applications. A kit may include one or more containers housing the components described herein and instructions for use. Specifically, such kits may include one or more agents described herein, along with instructions describing the intended application and the proper use of these agents. In certain embodiments agents in a kit may be in a pharmaceutical formulation and dosage suitable for a particular application and for a method of administration of the agents. Kits for research purposes may contain the components in appropriate concentrations or quantities for running various experiments.

The kit may be designed to facilitate use of the methods described herein by researchers and can take many forms. Each of the compositions of the kit, where applicable, may be provided in liquid form (e.g., in solution) or in solid form (e.g., a dry powder, a lyophilized powder). In certain cases, some of the compositions may be constitutable or otherwise processable (e.g., to an active form), for example, by the addition of a suitable solvent or other species (for example, water, buffered solution, or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, et cetera), internet, and/or web-based communications, et cetera The written instructions may be in a form prescribed by a governmental agency (e.g., US FDA or European Medicines Agency) regulating the manufacture, use or sale of pharmaceuticals or biological products, which instructions can also reflect approval by the agency of manufacture, use, or sale for animal administration and/or human use.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel, or solid (e.g., powder). The agents may be prepared sterilely, packaged in syringe, and shipped refrigerated. Alternatively, it may be housed in a vial or other container for storage. A second container may have other agents prepared sterilely. Alternatively, the kit may include the active agents premixed and shipped in a syringe, vial, tube, or other container.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### VI. General Techniques

The practice of the present invention will employ, unless otherwise indicated, conventional techniques of molecular biology (including recombinant techniques), microbiology, cell biology, biochemistry, and immunology, which are within the skill of the art. Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995)

Without further elaboration, it is believed that one skilled in the art can, based on the present disclosure, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

Exemplary embodiments of the invention will be described in more detail by the following examples. These embodiments are exemplary of the invention, which one skilled in the art will recognize is not limited to the exemplary embodiments.

The aspects described herein are not limited to specific embodiments, systems, compositions, methods, or configurations, and as such can, of course, vary. The terminology used herein is for the purpose of describing particular aspects only and, unless specifically defined herein, is not intended to be limiting.

### EXAMPLES

### Example 1: Regulation of Blood-CNS Barrier Permeability by Manipulating Ccdc141-Pacsin2 Interaction

### a Platform to Rapidly Knockdown Genes in a Brain EC in Adult Mice

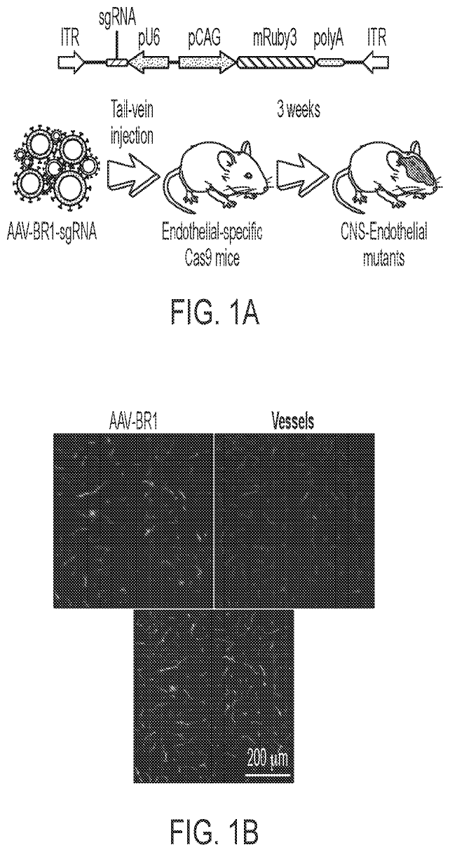

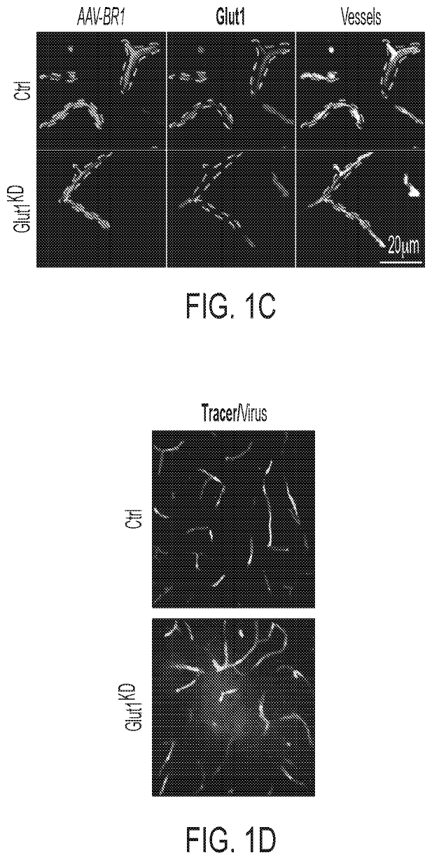

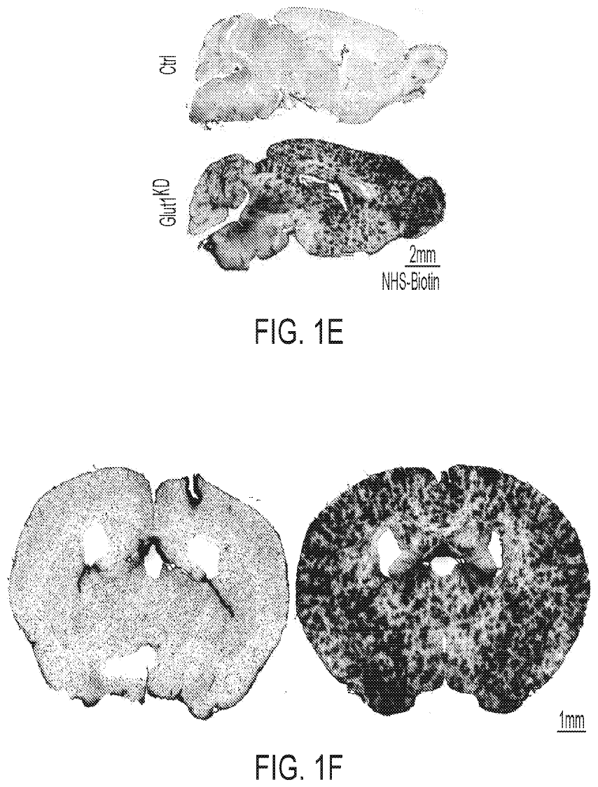

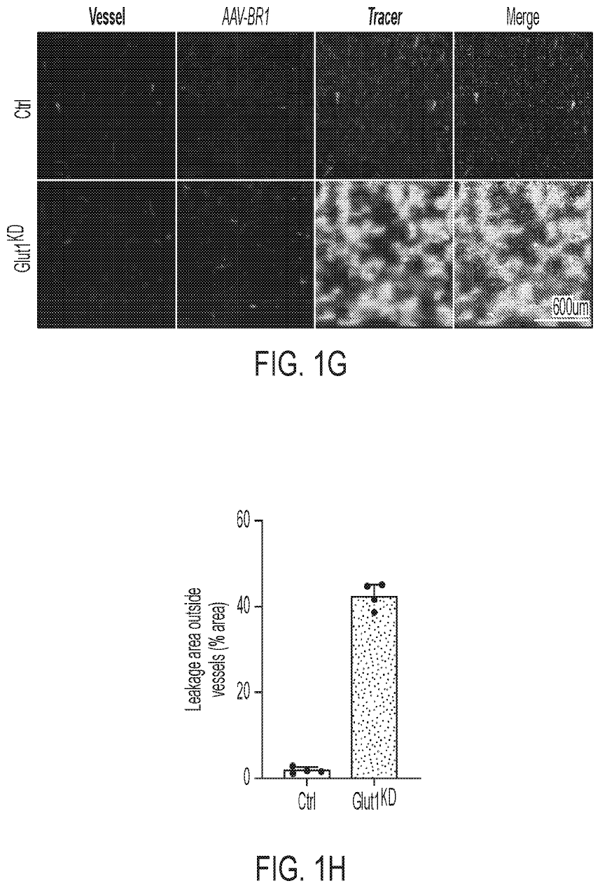

A brain endothelial-specific viral delivery platform approach was developed to acutely knock out candidate genes in adult mice. Specifically, a mouse line that expresses Cas9 in ECs was generated by crossing the Cre-dependent Cas9 mouse with Tie2:Cre+/− mice. Gene-specific singe-guide RNAs (sgRNAs) were delivered to central nervous system (CNS) endothelial cells in mice using adeno-associated virus (AAV) that specifically infect brain microvessels named AAV-BR1 which allowed acute knock out of any gene of interest in a CRISPR/Cas9 background (FIG. 1A). Injection of 5*e11 AAV-BR1 viral particles via tail-vein infected the majority (78%) of blood microvessels in the cortex of adult mice (FIG. 1B). This approach enhanced the ability to rapidly identify novel determinants of blood-brain barrier (BBB) integrity in vivo. The system was validated through selective ablation of the glucose transporter 1 (Glut1) in brain endothelial cells in adult mice. Glut1, encoded by Slc2a1, is expressed in brain ECs and is essential for neuronal survival and proper brain function and ablation of endothelial Glut1 in adult mice, results in a reduction of tight-junction proteins, reduced pericyte coverage and BBB breakdown. Examination of Glut1 expression using immunohistochemistry revealed that AAV-BR1-sgRNA:Slc2a1 successfully eliminated Glut1 expression specifically in infected cells (FIG. 1C) of Cre-dependent Cas9 mouse with Tie2: Cre+/− mice. To assess barrier function, mice were intravenously injected with sulfo-NHS-biotin, a small 556 daltons tracer prior to tissue harvesting. Examination of brain tissue revealed robust tracer extravasation throughout the brain of AAV-BR1-sgRNA:Slc2a1 injected mice while no leakage was observed in control mice (FIGS. 1D-1G). These results show that combining CRISPR/Cas9 with AAV-BR1 enable targeted loss-of-function genetic studies in brain endothelial to identify their roles in BBB maintenance in adult mice.

Transcriptome Analysis of CNS and Periphery ECs Identified Ccdc141, which is Highly Enriched in CNS ECs

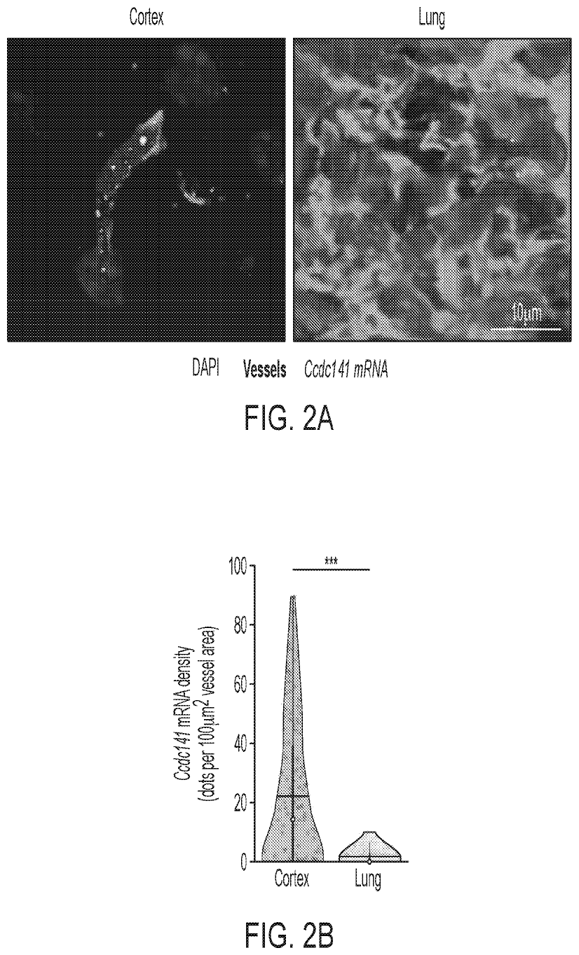

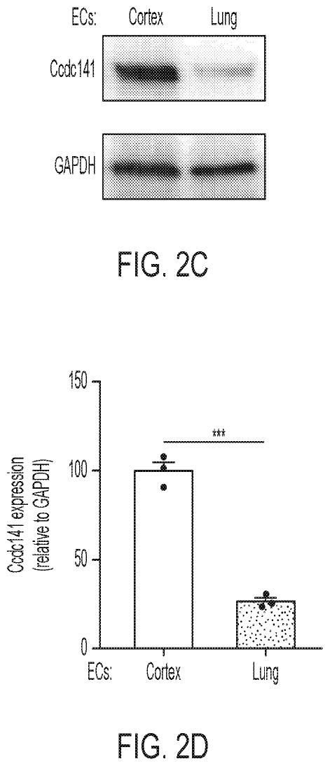

A comparative analysis of CNS and peripheral endothelial cells from published transcriptome datasets was performed to identify genes enriched at the BBB. Ccdc141 was identified as highly enriched in BBB endothelial cells compared to peripheral endothelial cells in all published datasets, including in newly formed blood-retina-barrier ECs. To confirm Ccdc141 enrichment in CNS vasculature, Ccdc141 mRNA localization in brain and lung was analyzed using commercially available RNAscope probes for Ccdc141 (FIGS. 2A-2B). Ccdc141 mRNA probes were specifically localized to ECs in the cortex and showed ~14-fold higher density compared to lung ECs (FIG. 2C). To validate Ccdc141 protein expression in CNS endothelial cells, a western-blot of purified endothelial prep from brain and lung tissues was performed. Ccdc141 protein concentrations in brain endothelial cells were four-times greater than lung endothelial cells (FIG. 2D). Together, these results confirm that Ccdc141 is expressed in CNS endothelial cells.

### Ccdc141 is Required for BBB Maintenance but not for Normal Vessel Patterning

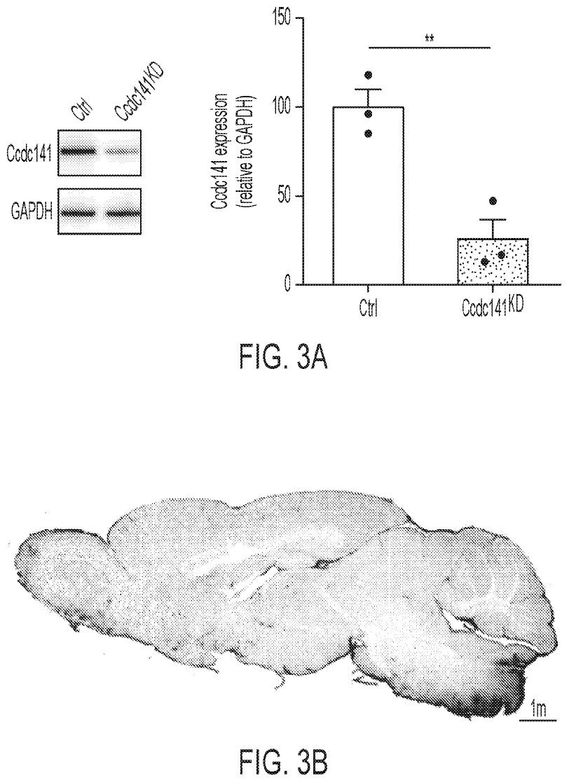

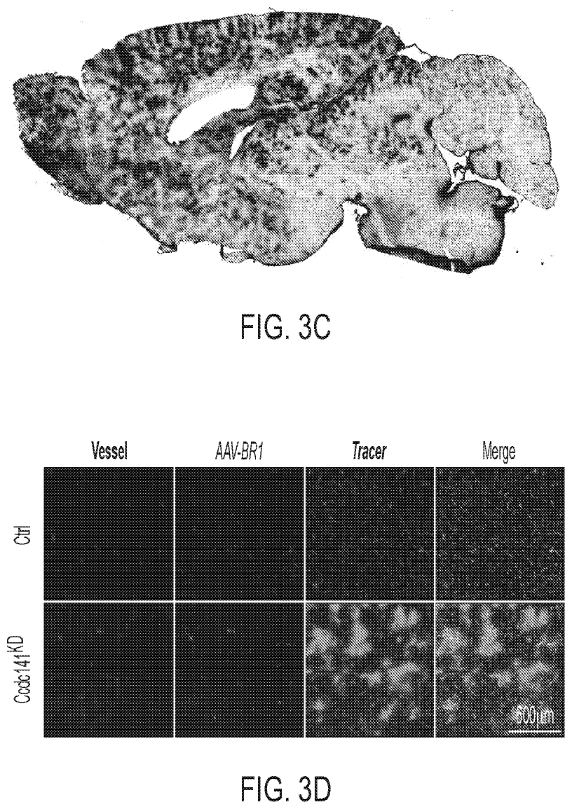

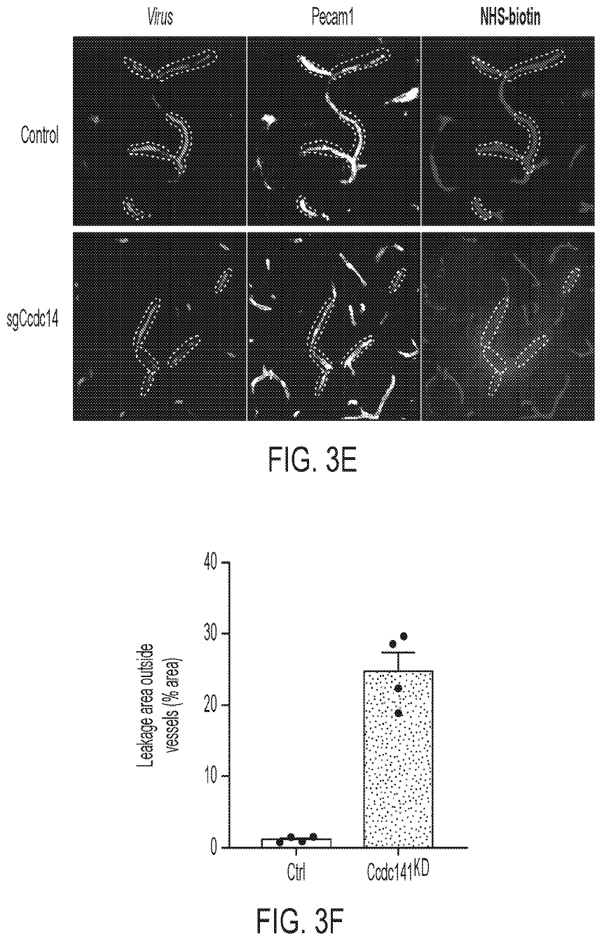

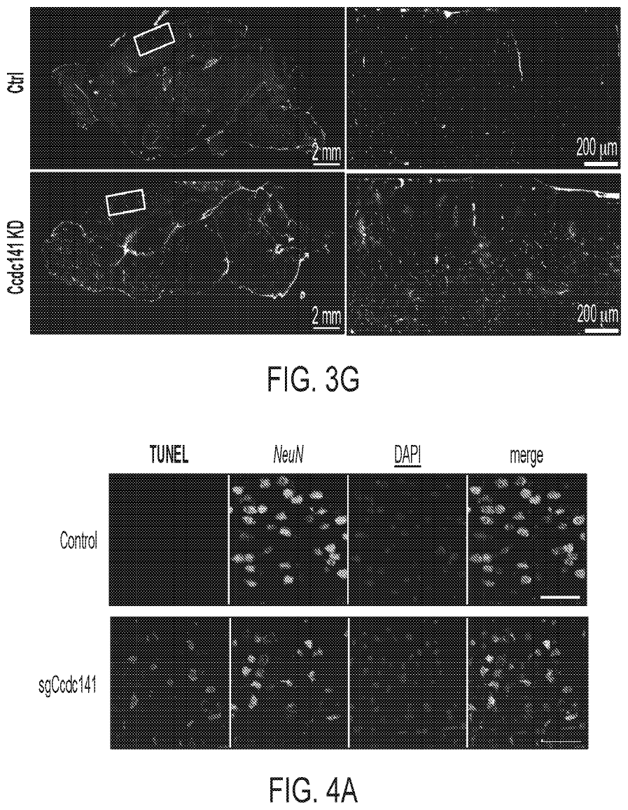

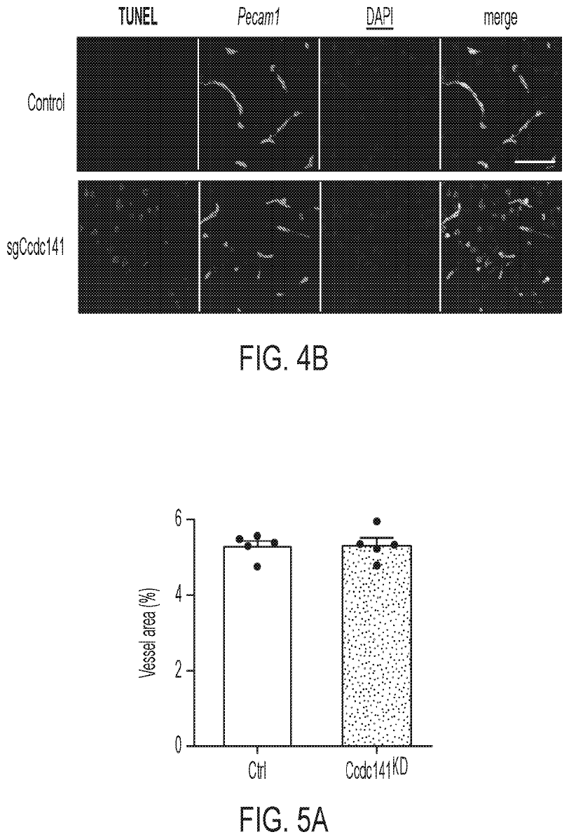

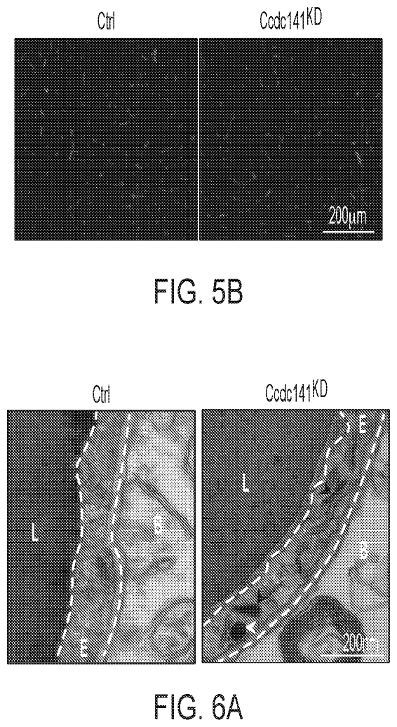

To examine whether Ccdc141 is important for BBB maintenance, Ccdc141 was knocked down using AAV-BR1 delivered sgRNAs targeting Ccdc141 in Cas9f/−; Tie2Cre and control (Cas9f/−) mice. Three weeks post viral injection, an 85% reduction in Ccdc141 protein level in brain capillary lysates from Ccdc141^{KD} (Ccdc141 knockdown) mice (AAV-BR1-sg Ccdc141; Cas9f/−; Tie2Cre) was observed relative to control animals (AAV-BR1-sg Ccdc141; Cas9f/−) (FIGS. 3A-3C). To determine the effects of endothelial-specific Ccdc141 reduction on blood-brain barrier function, sulfo-NHS-biotin was injected into Ccdc141KD mice prior to tissue harvesting. Ablation of Ccdc141 resulted in tracer extravasation in multiple brain areas including the cortex, thalamus, hippocampus, and olfactory bulb while in control animals the tracer was confined to blood vessels (FIG. 3D). Increased tracer leakage was observed specifically surrounding vessels of Ccdc141KD mice (FIG. 3E). Tracer extravasation in the cortex permeated ~23% of the parenchyma in sgCcdc141,Cre+ animals compared to less than 1% in control animals (FIG. 3F). Leakage was not limited to small tracers, as tracer extravasation was observed also using 10 kD dextran and 44 kD horse-radish peroxidase (HRP) and >100 kD endogenous IgG (FIG. 3G). Ccdc141 ablation in endothelial cells did not affect viability or other vascular functions. Ccdc141 ablation in neurons altered neuronal cell morphology and caused cell death without impacting vessel morphology (FIGS. 4A-4B). Vessel morphology and density was unchanged in Ccdc141KD mice relative to controls (FIG. 5A-5B). These data demonstrate that Ccdc141 is specifically required for BBB integrity but not for vessel patterning and health, indicating a direct role of Ccdc141 in barrier function.

### Ccdc141 Regulates BBB Function by Suppressing Tubular Vesicle Trafficking

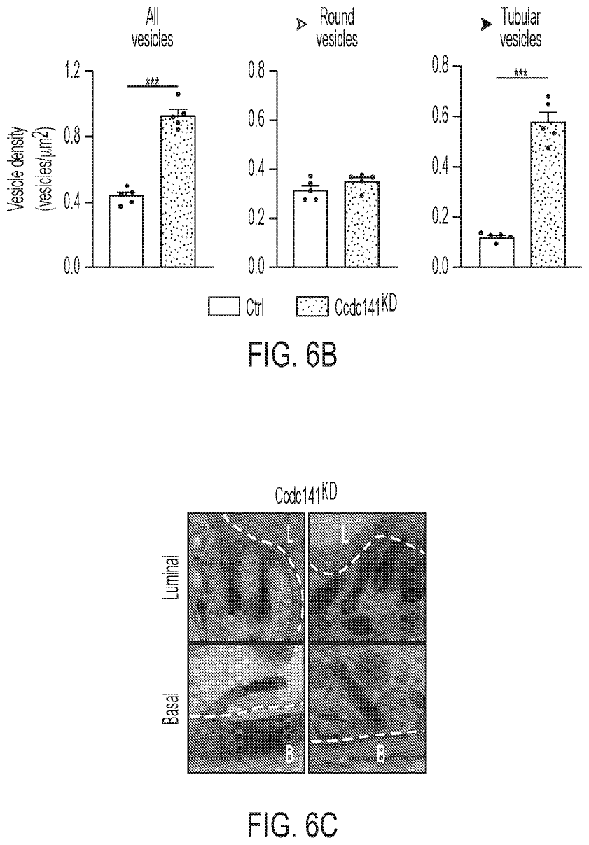

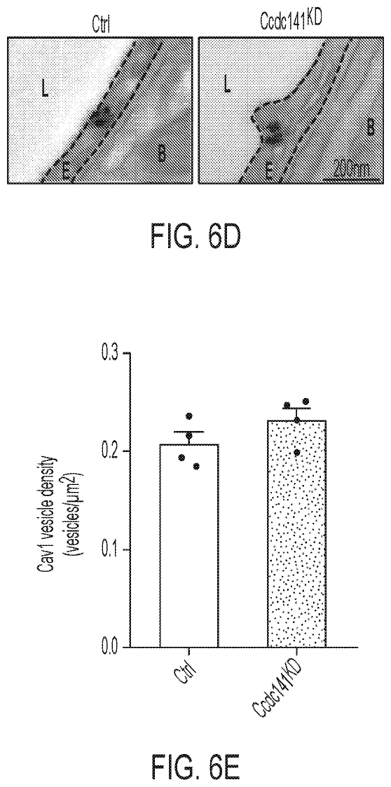

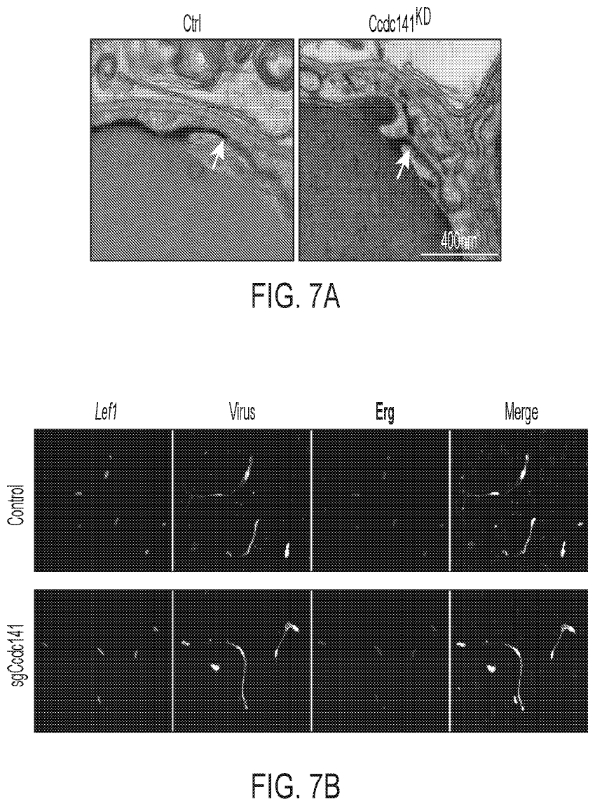

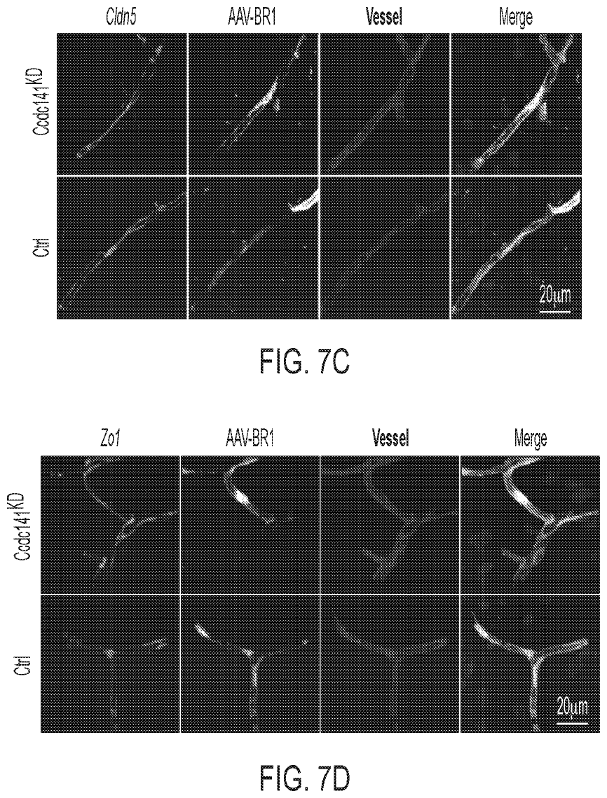

To understand the underlying subcellular cause for BBB leakage in sgCcdc141 KD animals, brain sections of HRP-iv injected animals were examined using electron microscopy (EM). Surprisingly, the HRP-filled vesicles were tubular in shape, in contrast to previously observed caveolae vesicles. There was a ~3-fold increase in tracer-filled vesicles in endothelial cells of sgCcdc141 injected animals compared to control animals (FIGS. 6A-6C). Caveolin-1 protein was not located in increased tracer-filled tubular vesicles observed in Ccdc141KD mice, as determined by immune-electron microscopy using caveolin-1 antibodies. As a control, caveolin-1 gold particles labeled robustly the caveolae vesicles in the arteriolar endothelial and neighboring smooth muscle cells (FIG. 6D-6E). Therefore, it was determined that Ccdc141 regulates tubular vesicle transcytosis is caveolae-independent. Ccdc141 KD and control mice exhibit functional tight junctions with 44 kDa HRP-and 1.9 kDa microperoxidase tracers successfully halted between endothelial cells. Expression of tight junction proteins including Cldn5, Zol (FIGS. 7C-7D) and Cadh5 were indistinguishable between sgCcdc141 RNA-treated and control mice.

### Ccdc141 Inhibits Pacsin2 Expression, a Known Tubular Vesicle Trafficking Regulator, in CNS ECs

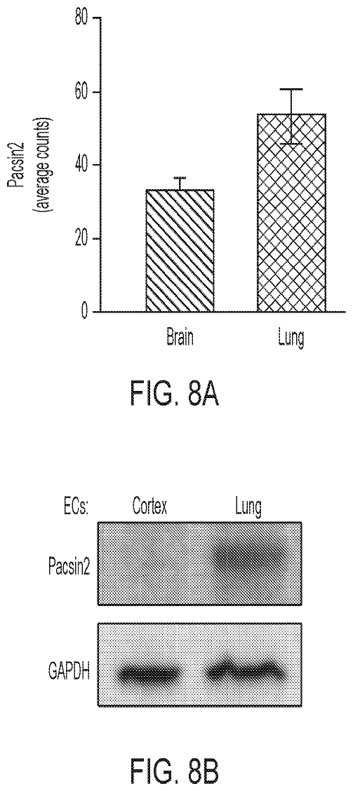

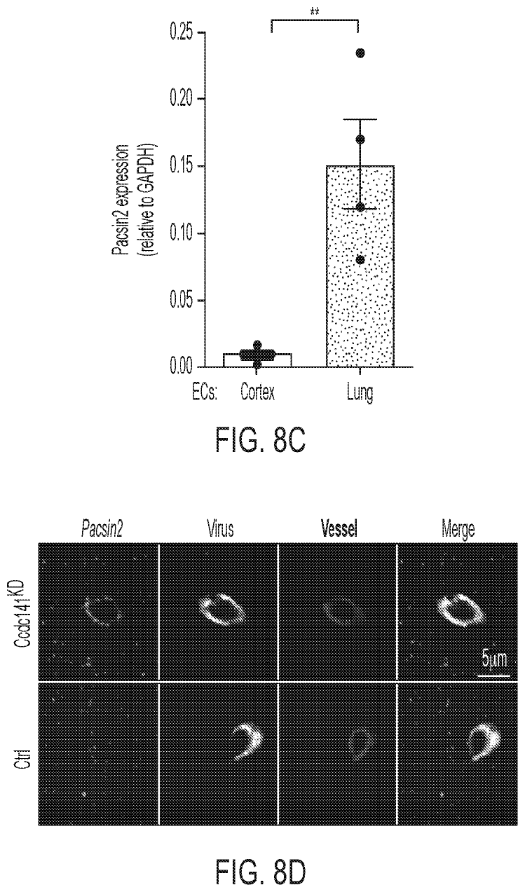

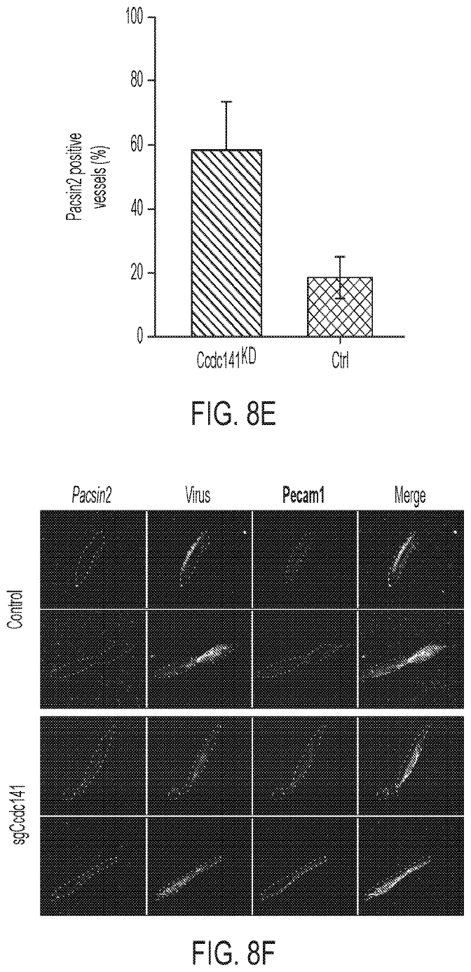

The family of BAR-domain containing proteins previously described as effectors in tubular shaped vesicle endocytosis were examined in response to Ccdc141 inhibition. Pacsin2 (protein kinase C and casein kinase substrate in neurons protein 2, also known as Syndapin-2), which are normally expressed at high levels in periphery endothelial cells and low levels in brain endothelial cells, was upregulated in the brain endothelial cells of Ccdc141 KD mice (FIGS. 8A-8F) indicating that a reduction Ccdc141 induces upregulation in Pacsin2 and subsequent formation of tubular transcytotic vesicles.

### Pacsin2 Over-Expression in CNS ECs is Sufficient to Cause BBB Leakage and Upregulation of Tubular Vesicle Transcytosis

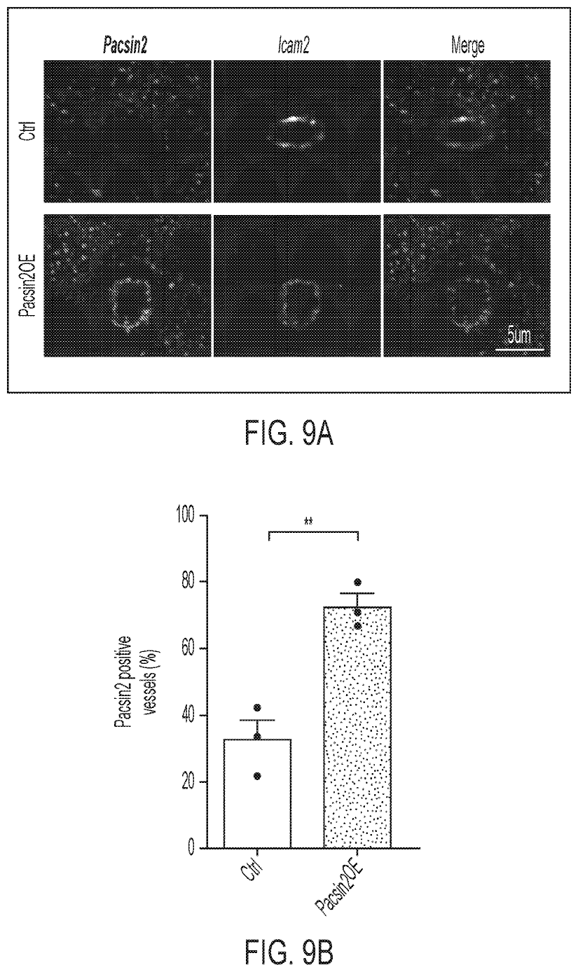

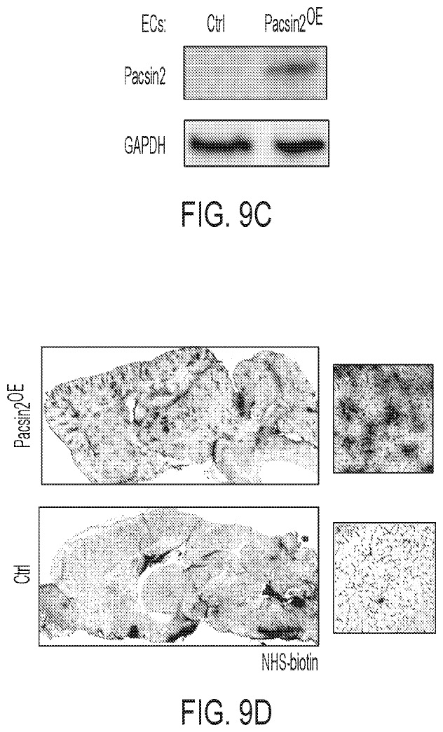

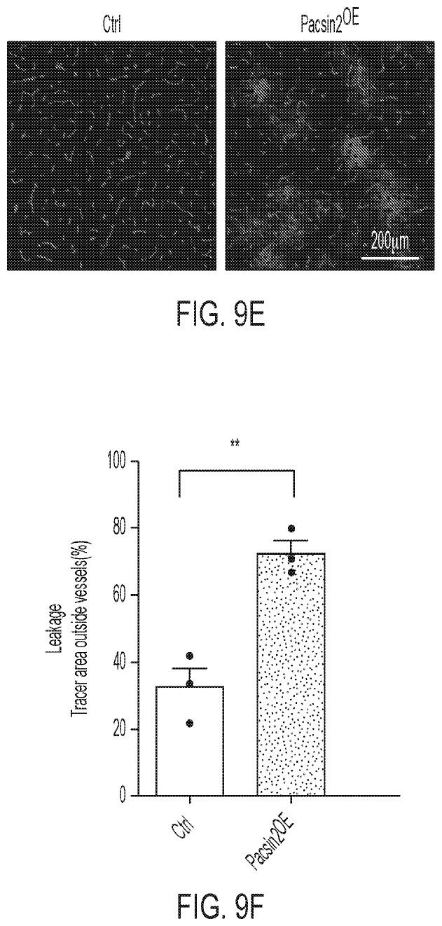

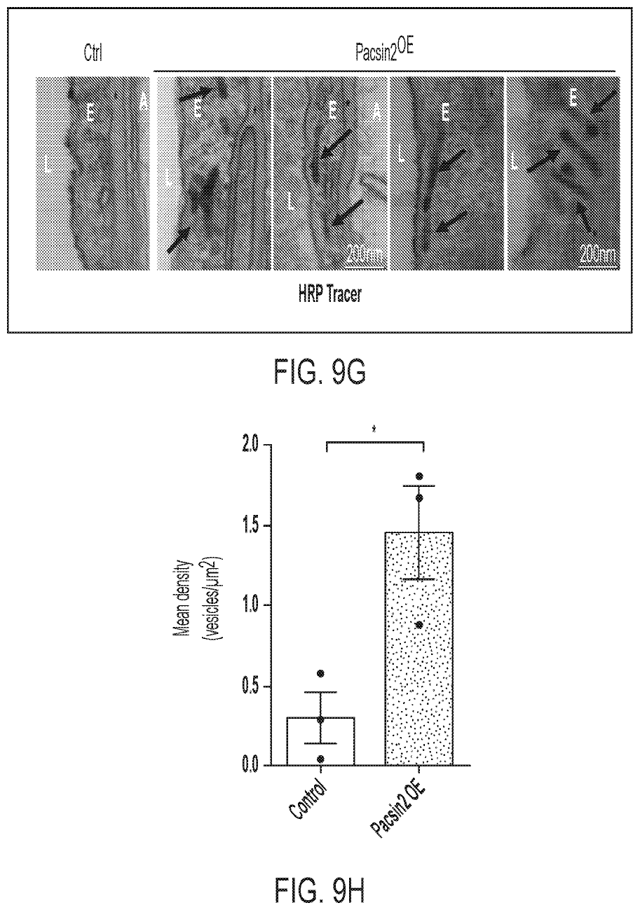

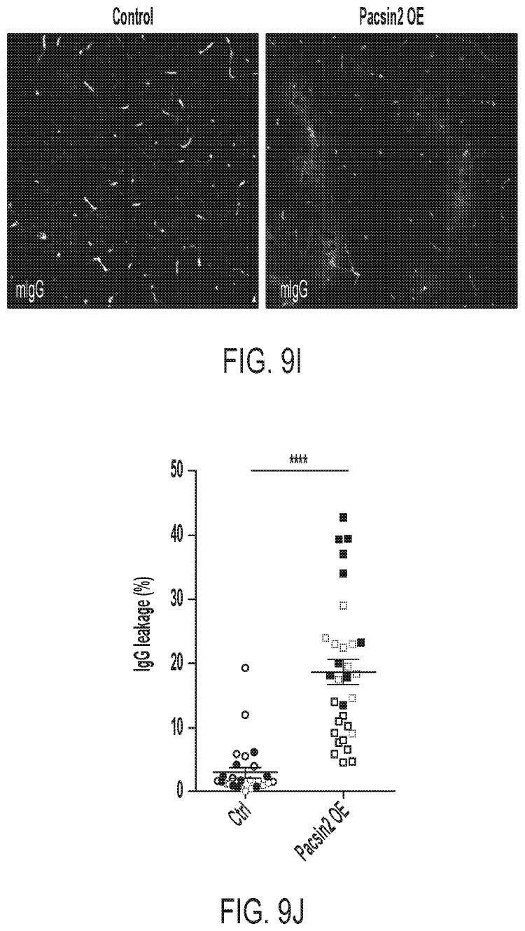

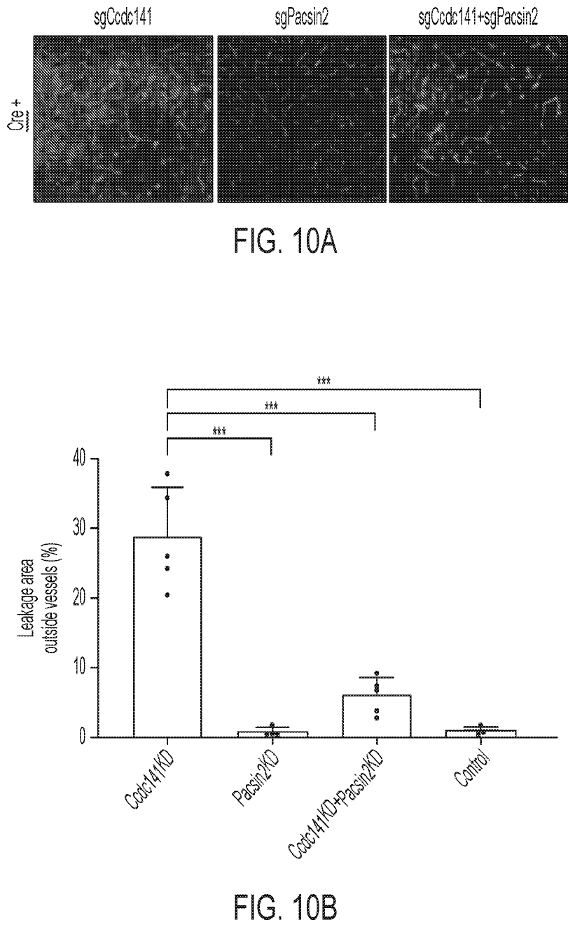

The effects of Pacsin2 over-expression were examined in the brain. AAV-BR1 expressing either Pacsin2 or mRuby3 fluorescent protein were iv injected into adult mice and sulfo-NHS-biotin leakage assays were performed after 3 weeks of the viral injection. Pacsin2 expression was increased in brain endothelial cells following injection with AAV-BR1 expressing Pacsin2, but not with mRuby3 (FIG. 9A-9C). Mice injected with AAV-Pacsin2 but not AAV-mRuby3 exhibited leakage in the brain (FIGS. 9D-9F) an increase in tubular vesicles in brain ECs in AAV-Pacsin2 injected mice but not in AAV-mRuby3 injected mice (FIG. 9G) demonstrating that upregulation of Pacsin2 in brain ECs was sufficient to increase BBB permeability and tubular vesicle-mediated transcytosis. The analysis of electron microscopy images of these brains showed that Pacsin2 overexpression results in increased tubular vesicles in CNS endothelial cells relative to controls (FIG. 9H). Further, leakage was not limited to small tracers, and endogenous IgG leakage was also observed in AAV-Pacsin2 injected mice but not in AAV-mRuby3 injected mice (FIGS. 91-9J). Ccdc141 regulates BBB by inhibiting Pacsin2 expression thus suppressing tubular vesicle mediated transcytosis Genetic rescue experiments were performed to determine if induced tubular vesicle transcytosis and BBB leakage were due to increased Pacsin2 expression in brain endothelial cells resulting from Ccdc141 knockdown. AAVs harboring the sgRNAs targeting Pacsin2 (sgPacsin2); Tie2Cre or its Cre-control mice were iv injected to Ccdc141 KD and control mice. sgCcdc141 injected animals exhibit upregulation of Pacsin2 in brain endothelial cells; however, sgPacsin2 and double infected animals did not exhibit elevated Pacsin2 immunoreactivity. BBB permeability was evaluated using sulfo-NHS-biotin tracer localization in the brain after intravenous injection. sgCcdc141 animals demonstrated increased tracer leakage, while an intact barrier was observed in sgPacsin2 injected mice. Mice that were infected with both sgCcdc141 and sgPacsin2 exhibited only mild leakage, wherein about 6% of the parenchyma was positive for the sulfo-NHS-biotin tracer (FIGS. 10A-10B). These findings suggest that upregulation in Pacsin2 is a crucial component in the leakage observed after Ccdc141 ablation in brain endothelial cells. Pacsin2 is expressed in aged human brain

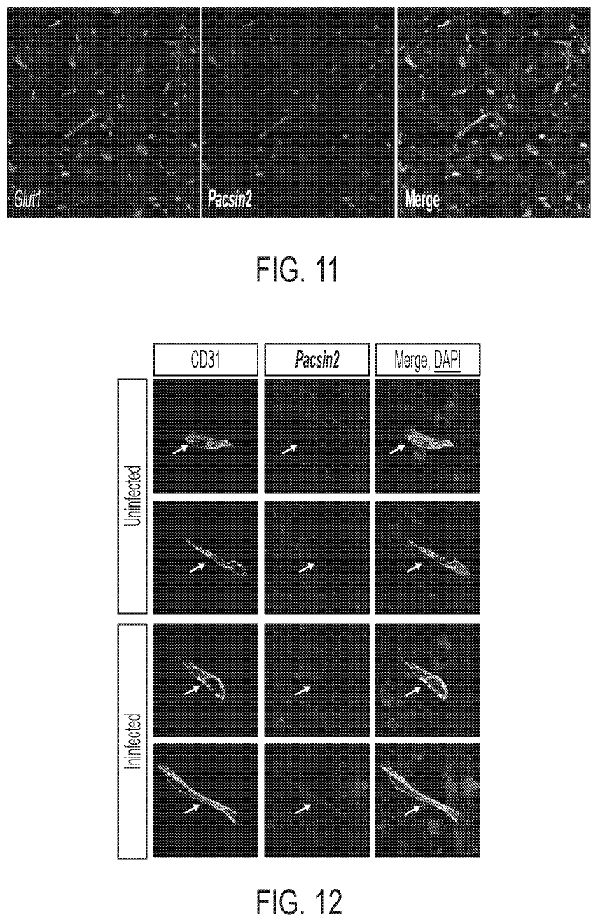

Cognitive impairment in aging and neurodegenerative disorders has been linked to a dysfunctional blood-brain barrier. The immunohistological examination of an aging human brain (78 years old) showed the expression of Pacsin2 in the blood vessels (FIG. 11).

### Pacsin2 is Upregulated in the HSV-Induced Neuroinflammation

Neuroinflammatory pathways are critical components in the pathogenesis of neurodegenerative diseases (e.g., ageing, early onset dementia). Pacsin2 protein levels were studied in mouse brains in a HSV-induced neuroinflammation model. Immunostained mouse cortical1 sections show that Pacsin2 protein was upregulated in endothelial cells of HSV-infected mice compared to uninfected mice (FIG. 12).

### REFERENCES

Ritter, B., Modregger, J., Paulsson, M., & Plomann, M. (1999). PACSIN 2, a novel member of the PACSIN family of cytoplasmic adapter proteins. FEBS letters, 454(3), 356-362.
Leite, D. M., Matias, D., & Battaglia, G. (2020). The role of BAR proteins and the glycocalyx in brain endothelium transcytosis. Cells, 9(12), 2685.
Hansen, C. G., Howard, G., & Nichols, B. J. (2011). Pacsin 2 is recruited to caveolae and functions in caveolar biogenesis. Journal of Cell Science, 124(16), 2777-2785.
Hutchins, B. I., Kotan, L. D., Taylor-Burds, C., Ozkan, Y., Cheng, P. J., Gurbuz, F., . . . & Wray, S. (2016). CCDCl41 mutation identified in anosmic hypogonadotropic hypogonadism (Kallmann syndrome) alters GnRH neuronal migration. Endocrinology, 157(5), 1956-1966.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the Claims.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following claims.

What is claimed is:

1. A method for increasing blood-Central Nervous System (blood-CNS) barrier permeability to treat a CNS disease in a subject, the method comprising administering the subject an inhibitor of Ccdc141 at the blood-CNS barrier.

2. The method of claim 1, wherein the blood-CNS barrier is the blood-brain barrier.

3. The method of claim 1, wherein the blood-CNS barrier is the blood-retina barrier.

4. The method of any one of claims 1-3, wherein the inhibitor of Ccdc141 is capable of inhibiting Ccdc141 expression and/or activity.

5. The method of any one of claims 1-4, wherein the inhibitor of Ccdc141 is an inhibitory nucleic acid targeting Ccdc141.

6. The method of claim 5, wherein the inhibitory nucleic acid targets the coding sequence of Ccdc141 comprising SEQ ID NO: 1 or SEQ ID NO: 2.

7. The method of claim 5, wherein the inhibitory nucleic acid targeting Ccdc141 is a siRNA, a shRNA, a miRNA, an AmiRNA, an aptamer, or an ASO.

8. The method of claim 7, wherein the inhibitory nucleic acid targeting Ccdc141 is a siRNA targeting Ccdc141.

9. The method of claim 8, wherein siRNA targeting Ccdc141 comprises an antisense strand complementary to any one of SEQ ID NOs: 9-21.

10. The method of claim 7, wherein the inhibitory nucleic acid targeting Ccdc141 is a shRNA targeting Ccdc141.

11. The method of claim 10, wherein the shRNA comprises a guide strand complementary to any one of SEQ ID NOs: 9-21.

12. The method of any one of claims 1-4, wherein inhibitor of Ccdc141 is an antibody, an antibody variant or an antigen-binding fragment targeting Ccdc141.

13. The method of any one of claims 1-4, wherein the inhibitor of Ccdc141 is a small molecule.

14. The method of any one of claims 1-13, wherein the inhibitor of Ccdc141 is delivered to CNS endothelial cells.

15. The method of any one of claims 1-14, wherein the inhibitory nucleic acid is delivered by a recombinant adeno-associated virus (rAAV).

16. The method of claim 15, wherein the capsid protein is BR1, BI30, AAV1, AAV2, AAV9, or variant thereof.

17. The method of claim 16, wherein the capsid protein is BR1.

18. The method of claim 16, wherein the capsid protein is BI30.

19. The method of any one of claims 1-18, wherein the inhibitor of Ccdc141 is administered to the subject via injection.

20. The method of claim 19, wherein the injection is systemic injection comprising intravenous injection, intramuscular injection, subcutaneous injection, or intraperitoneal injection.

21. The method of claim 19, wherein the injection is direct injection to the CNS comprising intracerebral injection, intraventricular injection, intracisternal injection, intraparenchymal injection, intrathecal injection, intravitreal injection, and any combination thereof.

22. A method for increasing Blood-Central Nervous System (Blood-CNS) Barrier permeability to treat a CNS disease in a subject, the method comprising administering an isolated nucleic acid comprising a transgene encoding Pacsin2.

23. The method of claim 22, wherein the transgene further comprises a promoter operably linked to a nucleotide sequence encoding Pacsin2.

24. The method of claim 22 or 23, wherein the isolated nucleic acid further comprises two adeno-associated virus inverted terminal repeats (ITRs) flanking the transgene encoding Pacsin2.

25. The method of any one of claims 22-24, wherein the isolated nucleic acid is administered to the subject via a recombinant adeno-associated virus (rAAV).

26. The method of claim 25, wherein the rAAV further comprises a capsid protein.

27. The method of claim 26, wherein the rAAV comprises a capsid protein comprising BR1, BI30, AAV1, AAV2, AAV9, or variant thereof.

28. The method of claim 27, wherein the capsid protein BR1 or BI30.

29. The method of any one of claims 1-28, further comprising administering a therapeutic agent for treating the CNS disease.

30. A method for delivering a therapeutic agent to the central nervous system to treat a CNS disease, the method comprising administering a subject:
(i) a therapeutic agent for treating the disease; and
(ii) an agent for increasing Blood-Central Nervous System (Blood-CNS) Barrier permeability.

31. The method of claim 30, wherein the agent for increasing Blood-CNS barrier permeability is an inhibitor of Ccdc141.

32. The method of claim 30, wherein the agent for increasing Blood-CNS barrier permeability is an isolated nucleic acid comprising a transgene encoding Pacsin2.

33. The method of any one of claims 1-32, wherein the CNS disease is a neuromuscular disease, neurodegenerative disease, brain and nerve tumor, neurogenetic disease, cognitive disorder, Familial dystonia, neuroinfectious disease, or neuropsychiatric disorder.

34. The method of claim 33, wherein the neuromuscular disease is Amyotrophic Lateral Sclerosis (ALS), Ataxia, Cerebral Palsy, or Muscular Dystrophy.

35. The method of claim 34, wherein the therapeutic agent is antibiotics, antibodies, anticonvulsants (e.g., gabapentin), chemotherapeutics, anti-inflammatoires, neurotransmitters, pain medication (e.g., morphine), peptides, nucleic acids (e.g., RNAi-based therapies), or psychiatric drugs.

36. The method of any one of claims 30-35, wherein the therapeutic agent is an antibody or a fragment thereof.

37. A method for decreasing Blood-Central Nervous System (Blood-CNS) Barrier permeability to treat a CNS disease in a subject, the method comprising administering the subject an inhibitor of Pacsin2 at the Blood-CNS Barrier.

38. The method of claim 37, wherein the blood-CNS barrier is the blood-brain barrier.

39. The method of claim 37, wherein the blood-CNS barrier is the blood-retina barrier.

40. The method of any one of claims 37-39, wherein the inhibitor of Pacsin2 is capable of inhibiting Pacsin2 expression and/or activity.

41. The method of any one of claims 37-40, wherein the inhibitor of Pacsin2 is an inhibitory nucleic acid targeting Pacsin2.

42. The method of claim 41, wherein the inhibitory nucleic acid targets the coding sequence of Pacsin2 comprising SEQ ID NOs: 5 and 6.

43. The method of claim 41 or 42, wherein the inhibitory nucleic acid targeting Pacsin2 is a siRNA, a shRNA, a miRNA, an AmiRNA, an aptamer, or an ASO.

44. The method of claim 43, wherein the inhibitory nucleic acid targeting Pacsin2 is a siRNA targeting Pacsin2.

45. The method of claim 44, wherein the siRNA targeting Pacsin2 comprises an antisense strand complementary to any one of SEQ ID NOs: 22-31, and 36-45.

46. The method of claim 43, wherein the inhibitory nucleic acid targeting Pacsin2 is a shRNA targeting Pacsin2.

47. The method of claim 46, wherein the shRNA targeting Pacsin2 comprises a guide strand complementary to any one of SEQ ID NOs: 22-31, and 36-45.

48. The method of any one of claims 37-40, wherein inhibitor of Pacsin2 is an antibody, an antibody variant or an antigen-binding fragment targeting Pacsin2.

49. The method of any one of claims 37-40, wherein the inhibitor of Pacsin2 is a small molecule.

50. The method of any one of claims 37-49, wherein the inhibitor of Pacsin2 is delivered to CNS endothelial cells.

51. The method of any one of claims 37-50, wherein the inhibitor of Pacsin2 is delivered by a recombinant adeno-associated virus (rAAV).

52. The method of claim 51, wherein the rAAV comprises a capsid protein comprising BR1, BI30, AAV1, AAV2, AAV9, or variant thereof.

53. The method of claim 52, wherein the capsid protein is BR1.

54. The method of claim 52, wherein the capsid protein is BI30.

55. The method of any one of claims 37-54, wherein the inhibitor of Pacsin2 is administered to the subject via injection.

56. The method of claim 55, wherein the injection is systemic injection comprising intravenous injection, intramuscular injection, subcutaneous injection, or intraperitoneal injection.

57. The method of claim 55, wherein the injection is direct injection to the CNS comprising intracerebral injection, intraventricular injection, intracisternal injection, intraparenchymal injection, intrathecal injection, and any combination thereof.

58. A method for decreasing Blood-Central Nervous System (Blood-CNS) Barrier permeability to treat a CNS disease in a subject, the method comprising administering an isolated nucleic acid comprising a transgene encoding Ccdc141.

59. The method of claim 58, wherein the transgene further comprises a promoter operably linked to a nucleotide sequence encoding Ccdc141.

60. The method of claim 58 or 59, wherein the isolated nucleic acid further comprises two adeno-associated virus inverted terminal repeats (ITRs) flanking the transgene encoding Ccdc141.

61. The method of any one of claims 58-60, wherein the isolated nucleic acid is administered to the subject via a recombinant adeno-associated virus (rAAV).

62. The method of claim 61, wherein the rAAV further comprises a capsid protein.

63. The method of claim 62, wherein the capsid protein is BR1, BI30, AAV1, AAV2, AAV9, or variant thereof.

64. The method of claim 63, wherein the capsid protein is BR1.

65. The method of claim 63, wherein the capsid protein is BI30.

66. The method of any one of claims 58-65, wherein the CNS disease is retinal disease, neurodegenerative disease, acute injury of the CNS, neuroinfectious disease, primary and metastatic cancers of the CNS, autoimmune disease of the CNS, neuroinflammatory conditions, or cognitive disorder.

67. The method of claim 66, wherein the retinal disease is diabetic retinopathy.

68. The method of claim 66, wherein the neurodegenerative disease is Huntington's disease.

69. The method of claim 66, wherein the acute injury of the CNS is stroke or head trauma.

70. The method of claim 66, wherein the Neuroinfectious disease is encephalitis, sepsis, or COVID-19.

71. The method of claim 66, wherein the primary cancer of the CNS is glioblastoma, meningioma, retinoblastoma, or lymphoma.

72. The method of claim 66, wherein the metastatic cancer of the CNS is lung cancer, metastatic breast cancer, or melanoma.

73. The method of claim 66, wherein the autoimmune disease of the CNS is multiple sclerosis.

74. The method of claim 66, wherein the neuroinflammatory condition is CNS Lupus, CNS Lyme Disease, Neurosarcoidosis, Neuromyelitis optica (NMO), or Paraneoplastic and Autoimmune Encephalitis.

75. The method of claim 66, wherein the cognitive disorder is dementia resulting from Alzheimer's disease, Lewy body dementia, frontotemporal dementia, encephalopathy, or post-acute COVID syndrome.

76. A method for treating a condition associated with increased blood-CNS barrier permeability, the method comprising administering a subject a Pacsin2 inhibitor or an agent promoting Ccdc141 expression/activity.

77. The method of claim 76, wherein the condition is associated with aging.

78. The method of claim 76 or 77, wherein the condition is early onset dementia.

79. The method of claim 76, wherein the condition is stroke.

80. The method of claim 76, wherein the condition is spinal cord injury.

81. Use of a Pacsin2 inhibitor or an agent promoting Ccdc141 expression/activity for treating a condition associated with increased blood-CNS barrier permeability.

82. Use of an inhibitor of Ccdc141 at the blood-CNS barrier for increasing blood-Central Nervous System (blood-CNS) barrier permeability to treat a CNS disease in a subject.

83. Use of an isolated nucleic acid comprising a transgene encoding Pacsin2 for increasing Blood-Central Nervous System (Blood-CNS) Barrier permeability to treat a CNS disease in a subject.

84. Use of a therapeutic agent for treating the disease in combination with an agent for increasing Blood-Central Nervous System (Blood-CNS) Barrier permeability for delivering a therapeutic agent to the central nervous system to treat a CNS disease.

85. Use of an inhibitor of Pacsin2 at the Blood-CNS Barrier for decreasing Blood-Central Nervous System (Blood-CNS) Barrier permeability to treat a CNS disease in a subject.

86. Use of an isolated nucleic acid comprising a transgene encoding Ccdc141 for decreasing Blood-Central Nervous System (Blood-CNS) Barrier permeability to treat a CNS disease in a subject.
